SEQUENCE LISTING (94 sequences)

SEQ ID NO: 1 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 2 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 3 - protein, 258 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 4 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 5 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 6 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 7 - protein, 249 aa, Thermoascus aurantiacus
MSFSKIIATAGVLASASLVAGHGFVQNIVIDGKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 8 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 9 - protein, 232 aa, Myceliophthora thermophila
MKFTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIDDGEYLLRVEHIALHSASSVGGAQFYIACAQLSVTGGSGTLNTGSLVSLPGAYKATDPGILFQLYWPIPTEYINPGPAPVSC

SEQ ID NO: 10 - protein, 235 aa, Myceliophthora thermophila
MKALSLLAAASAVSAHTIFVQLEADGTRYPVSYGIRDPSYDGPITDVTSNDVACNGGPNPTTPSSDVITVTAGTTVKAIWRHTLQSGPDDVMDASHKGPTLAYLKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 11 - protein, 323 aa, Myceliophthora thermophila
MKSFALTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGEGYTGCTNCASGSTCSAVSPPYYSQCV

SEQ ID NO: 12 - protein, 310 aa, Myceliophthora thermophila
MKPFSLVALATAVSGHAIFQRVSVNGQDQGQLKGVRAPSSNSPIQNVNDANMACNANIVYHDSTIIKVPAGARVGAWWQHVIGGPQGANDPDNPIAASHKGPIQVYLAKVDNAATASPSGLRWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPTNGGRSYPIPGPRPISCSGSGDGGNNGGGGDDNNNNNGGGNNGGGGGGSVPLYGQCGGIGYTGPTTCAQGTCKVSNEYYSQCLP

SEQ ID NO: 13 - protein, 246 aa, Myceliophthora thermophila
MKLSLFSVLATALTVEGHAIFQKVSVNGADQGSLTGLRAPNNNNPVQDVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPPSTVSFPGAYSASDPGILINIYGATGQPDNNGQPYTAPGPAPISC

SEQ ID NO: 14 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 15 - protein, 250 aa, Aspergillus fumigatus [sequence omitted]

SEQ ID NO: 16 - protein, 322 aa, Penicillium pinophilum
MPSTKVAALSAVLALASTVAGHGFVQNIVIDGKSYSGYLVNQFPYESNPPAVIGWATTATDLGFVAPSEYTNADIICHKNATPGALSAPVAAGGTVELQWTTWPDSHHGPVISYLANCNGNCSTVDKTKLDFVKIDQGGLIDDTTPPGTWASDKLIAANNSWTVTIPSTIAPGNYVLRHEIIALHSAGNADGAQNYPQCINLEITGSGTAAPSGTAGEKLYTSTDPGILVNIYQSLSTYVIPGPTLWSGAANGAVATGSATAVATTATASATATPTTLVTSVAPASSTFATAVVTTVAPAVTDVVTVTDVVTVTTVITTTVL

SEQ ID NO: 17 - protein, 444 aa, Thermoascus sp.
MLSFASAKSAVLTTLLLLGSAQAHTLMTTLFVDGVNQGDGVCIRMNNNGSTANTYIQPVTSKDIACGIQGEIGAARVCPAKASSTLTFQFREQPSNPNSAPLDPSHKGPAAVYLKKVDSAIASNNAAGDGWFKIWESVYDESTGKWGTTKMIENNGHISVKVPDDIEGGYYLARTELLALHAANEGDPQFYVGCAQLFIDSAGTAKPPTVSIGEGTYDLSMPAMTYNIYQTPLALPYPMYGPPVYTPGSGSGSGSGSGSASATRSSAIPTATAVTDCSSEEDREDSVMATGVPVARSTLRTWVDRLSWHGKARENVKPAARRSALVQTEGLKPEGCIFVNGNWCGFEVPDYNDAESCWAASDNCWKQSDSCWNQTQPTGYNNCQIWQDQKCKPIQDSCSQSNPTGPPNKGKDITPTWPPLEGSMKTFTKRTVSYRDWIMKRKGA

SEQ ID NO: 18 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 19 - protein, 246 aa, Thielavia terrestris
MKFSLVSLLAYGLSVEAHSIFQRVSVNGQDQGLLTGLRAPSNNNPVQDVNSQNMICGQSGSKSQTVINVKAGDRIGSLWQHVIGGAQFSGDPDNPIAHSHKGPVMAYLAKVDNAASASQTGLKWFKIWQDGFDTSSKTWGVDNLIKNNGWVYFHLPQCLAPGQYLLRVEVLALHSAYQQGQAQFYQSCAQINVSGSGSFSPSQTVSIPGVYSATDPSILINIYGSTGQPDNGGKAYNPPGPAPISC

SEQ ID NO: 20 - protein, 334 aa, Thielavia terrestris
MRTTFAAALAAFAAQEVAGHAIFQQLWHGSSCVRMPLSNSPVTNVGSRDMICNAGTRPVSGKCPVKAGGTVTVEMHQQPGDRSCNNEAIGGAHWGPVQVYLSKVEDASTADGSTGWFKIFADTWSKKAGSSVGDDDNWGTRDLNACCGKMQVKIPADIPSGDYLLRAEALALHTAGQVGGAQFYMSCYQITVSGGGSASPATVKFPGAYSANDPGIHINIHAAVSNYVAPGPAVYSGGTTKVAGSGCQGCENTCKVGSSPTATAPSGKSGAGSDGGAGTDGGSSSSSPDTGSACSVQAYGQCGGNGYSGCTQCAPGYTCKAVSPPYYSQCAPSS

SEQ ID NO: 21 - protein, 227 aa, Thielavia terrestris
MKLSVAIAVLASALAEAHYTFPSIGNTADWQYVRITTNYQSNGPVTDVTSDQIRCYERNPGTGAQGIYNVTAGQTINYNAKASISHPGPMSFYIAKVPAGQTAATWDGKGAVWTKIYQDMPKFGSSLTWPTMGAKSVPVTIPRCLQNGDYLLRAEHIALHSASSVGGAQFYLSCAQLTVSGGSGTWNPKNRVSFPGAYKATDPGILINIYYPVPTSYSPPGPPAETC

SEQ ID NO: 22 - protein, 223 aa, Thielavia terrestris
MKLSSQLAALTLAAASVSGHYIFEQIAHGGTKFPPYEYIRRNTNYNSPVTSLSSNDLRCNVGGETAGNTTVLDVKAGDSFTFYSDVAVYHQGPISLYMSKAPGSVVDYDGSGDWFKIHDWGPTFSNGQASWPLRDNYQYNIPTCIPNGEYLLRIQSLAIHNPGATPQFYISCAQVRVSGGGSASPSPTAKIPGAFKATDPGYTANIYNNFHSYTVPGPAVFQC

SEQ ID NO: 23 - protein, 368 aa, Thielavia terrestris
MPSFASKTLLSTLAGAASVAAHGHVSNIVINGVSYQGYDPTSFPYMQNPPIVVGWTAADTDNGFVAPDAFASGDIICHKNATNAKGHAVVAAGDKIFIQWNTWPESHHGPVIDYLASCGSASCETVDKTKLEFFKIDEVGLVDGSSAPGVWGSDQLIANNNSWLVEIPPTIAPGNYVLRHEIIALHSAENADGAQNYPQCFNLQITGTGTATPSGVPGTSLYTPTDPGILVNIYSAPITYTVPGPALISGAVSIAQSSSAITASGTALTGSATAPAAAAATTTSTTNAAAAATSAAAAAGTSTTTTSAAAVVQTSSSSSSAPSSAAAAATTTAAASARPTGCSSGRSRKQPRRHARDMVVARGAEEAN

SEQ ID NO: 24 - protein, 330 aa, Thielavia terrestris
MPPALPQLLTTVLTALTLGSTALAHSHLAYIIVNGKLYQGFDPRPHQANYPSRVGWSTGAVDDGFVTPANYSTPDIICHIAGTSPAGHAPVRPGDRIHVQWNGWPVGHIGPVLSYLARCESDTGCTGQNKTALRWTKIDDSSPTMQNVAGAGTQGEGTPGKRWATDVLIAANNSWQVAVPAGLPTGAYVLRNEIIALHYAARKNGAQNYPLCMNLWVDASGDNSSVAATTAAVTAGGLQMDAYDARGFYKENDPGVLVNVTAALSSYVVPGPTVAAGATPVPYAQQSPSVSTAAGTPVVVTRTSETAPYTGAMTPTVAARMKGRGYDRRG

SEQ ID NO: 25 - protein, 236 aa, Thielavia terrestris
MKTFTALLAAAGLVAGHGYVDNATIGGQFYQNPAVLTFFQPDRVSRSIPGNGPVTDVTLIDLQCNANSTPAKLHATAAAGSDVILRWTLWPESHVGPVITYMARCPDTGCQDWMPGTSAVWFKIKEGGRDGTSNTWADTPLMTAPTSYTYTIPSCLKKGYYLVRHEIIALHAAYTYPGAQFYPGCHQLNVTGGGSTVPSSGLVAFPGAYKGSDPGITYDAYKAQTYQIPGPAVFTC

SEQ ID NO: 26 - protein, 250 aa, Thielavia terrestris
MALLLLAGLAILAGPAHAHGGLANYTVGNTWYRGYDPFTPAADQIGQPWMIQRAWDSIDPIFSVNDKALACNTPATAPTSYIPIRAGENITAVYWYWLHPVGPMTAWLARCDGDCRDADVNEARWFKIWEAGLLSGPNLAEGMWYQKAFQNWDGSPDLWPVTIPAGLKSGLYMIRHEILSIHVEDKPQFYPECAHLNVTGGGDLLPPDEFLVKFPGAYKEDNPSIKINIYSDQYANTTNYTIPGGPIWDG

SEQ ID NO: 27 - protein, 478 aa, Thielavia terrestris
MMPSLVRFSMGLATAFASLSTAHTVFTTLFINGVDQGDGTCIRMAKKGSVCTHPIAGGLDSPDMACGRDGQQAVAFTCPAPAGSKLSFEFRMWADASQPGSIDPSHLGSTAIYLKQVSNISSDSAAGPGWFKIYAEGYDTAAKKWATEKLIDNGGLLSIELPPTLPAGYYLARSEIVTIQNVTNDHVDPQFYVGCAQLFVQGPPTTPTVPPDRLVSIPGHVHASDPGLTFNIWRDDPSKTAYTVVGPAPFSPTAAPTPTSTNTNGQQQQQQQQAIKQTDGVIPADCQLKNANWCGAEVPAYADEAGCWASSADCFAQLDACYTSAPPTGSRGCRLWEDWCTGIQQGCRAGRWRGPPPFHGEGAAAETASAGRGGARIAAVAGCGGGTGDMVEEVFLFYWDACSGWRRSRGGGSILARLILHVLLPLLRPRRAPRVHLLLFHLYLNFCYPGTSGFYNRLSIKLGIWPSKMSPDVAHYVK

SEQ ID NO: 28 - protein, 230 aa, Thielavia terrestris
MQLLVGLLLAAVAARAHYTFPRLVVNGQPEDKDWSVTRMTKNAQSKQGVQDPTSPDIRCYTSQTAPNVATVPAGATVHYISTQQINHPGPTQYYLAKVPAGSSAKTWDGSGAVWFKISTTMPYLDNNKQLVWPNQNTYTTVNTTIPADTPSGEYLLRVEQIALHLASQPNGAQFYLACSQIQITGGGNGTPGPLVALPGAYKSNDPGILVNIYSMQPGDYKPPGPPVWSG

SEQ ID NO: 29 - protein, 257 aa, Thielavia terrestris
MKLYLAAFLGAVATPGAFAHQIHGILLVNGTETPEWKYVRDVAWEGAYEPEKYPNTEFFKTPPQTDINNPNITCGRNAFDSASKTETADILAGSEVGFRVSWDGNGKYGVFWHPGPGQIYLSRAPNDDLEDYRGDGDWFKIATGAAVSNTEWLLWNKHDFNFTIPKTTPPGKYLMRIEQFMPSTVEYSQWYVNCAHVNIIGPGGGTPTGFARFPGTYTVDDPGIKVPLNQIVNSGELPQDQLRLLEYKPPGPALWTG

SEQ ID NO: 30 - protein, 251 aa, Thermoascus crustaceus
MAFSQIMAITGVFLASASLVAGHGFVQNIVIDGKSYGGYIVNQYPYMSDPPEVVGWSTTATDLGFVDGTGYQGPDIICHRGAKPAALTAQVAAGGTVKLEWTPWPDSHHGPVINYLAPCNGDCSTVDKTQLKFFKIAQAGLIDDNSPPGIWASDNLIAANNSWTVTIPTTTAPGNYVLRHEIIALHSAGNKDGAQNYPQCINLKVTGNGSGNPPAGALGTALYKDTDPGILINIYQKLSSYVIPGPALYTG

SEQ ID NO: 31 - protein, 349 aa, Thermoascus crustaceus
MSFSKILAIAGAITYASSAAAHGYVQGIVVDGSYYGGYMVTQYPYTAQPPELIAWSTKATDLGFVDGSGYTSPDIICHKGAEPGAQSAKVAAGGTVELQWTAWPESHKGPVIDYLAACDGDCSSVDKTALKFFKIDESGLIDGNGAGTWASDTLIKNNNSWTVTIPSTIASGNYVLRHEIIALHSAGNKDGAQNYPQCINLEVTGSGTENPAGTLGTALYTDTDPGLLVNIYQGLSNYSIPGPALYSGNSDNAGSLNPTTTPSIQNAAAAPSTSTASVVTDSSSATQTASVAATTPASTSAVTASPAPDTGSDVTKYLDSMSSDEVLTLVRGTLSWLVSNKKHARDLSH

SEQ ID NO: 32 - protein, 436 aa, Thermoascus crustaceus
MLSFIPTKSAALTTLLLLGTAHAHTLMTTMFVDGVNQGDGVCIRMNNDGGTANTYIQPITSKDIACGIQGEIGASRVCPVKASSTLTFQFREQPNNPNSSPLDPSHKGPAAVYLKKVDSAIASNNAAGDSWFKIWESVYDESTGKWGTTKMIENNGHISVKVPDDIEGGYYLARTELLALHSADQGDPQFYVGCAQLFIDSDGTAKPPTVSIGEGTYDLSMPAMTYNIWETPLALPYPMYGPPVYTPGSGSGSVRATSSSAVPTATESSFVEERANPVTANSVYSARGKFKTWIDKLSWRGKVRENVRQAAGRRSTLVQTVGLKPKGCIFVNGNWCGFEVPDYNDAESCWAASDNCWKQSDACWNKTQPTGYNNCQIWQDKKCKVIQDSCSGPNPHGPPNKGKDLTPEWPPLKGSMDTFSKRTIGYRDWIVRRRGA

SEQ ID NO: 33 - protein, 344 aa, Aspergillus aculeatus
MKYIPLVIAVAAGLARPATAHYIFSKLVLNGEASADWQYIRETTRSIVYEPTKYTSTFDNLTPSDSDFRCNLGSFSNAAKTEVAEVAAGDTIAMKLFYDTSIAHPGPGQVYMSKAPTGNVQEYQGDGDWFKIWEKTLCNTDGDLTTEAWCTWGMSQFEFQIPAATPAGEYLVRAEHIGLHGAQANEAEFFYSCAQIKVTGSGTGSPSLTYQIPGLYNDTMTLFNGLNLWTDSAEKVQLDFLETPIGDDVWSGAGSGSPSAATSSTSGATLAAQGTTTSAAHAQAQTTITTSTSTITSLESASSTDLVAQYGQCGGLNWSGPTECETPYTCVQQNPYYHQCVNSC

SEQ ID NO: 34 - protein, 400 aa, Aspergillus aculeatus
MSVAKFAGVILGSAALVAGHGYVSGAVVDGTYYGGYIVTSYPYSSDPPETIGWSTEATDLGFVDGSEYADADIICHKSAKPGAISAEVKAGGTVELQWTTWPDSHHGPVLTYLANCNGDCSSVTKTDLEFFKIDESGLINDDDVPGTWASDNLIANNNSWTVTIPSDIAAGNYVLRHEIIALHSAGNKDGAQNYPQCLNLKVTGGGDLAPSGTAGESLYKDTDAGILVNIYQSLSSYDIPGPAMYNATSSSSSSSSSSSSSSSSSSSGSSSSAAASSSSSSSSTTAAAAAATSAASSVTSAAGSVVTQTATAVETDTATAYQTSTEVAQVTVTGSAPQQTYVATPSSSSSASSSSSASVSTSTSLTSYFESLSADQFLSVLKQTFTWLVSEKKHARDLSA

SEQ ID NO: 35 - protein, 389 aa, Aspergillus aculeatus
MKSSTFGMLALAAAAKMVDAHTTVFAVWINGEDQGLGNSASGYIRSPPSNSPVKDVTSTDITCNVNGDQAAAKTLSVKGGDVVTFEWHHDSRDASDDIIASSHKGPVMVYMAPTTAGSSGKNWVKIAEDGYSDGTWAVDTLIANSGKHNITVPDVPAGDYLFRPEIIALHEAENEGGAQFYMECVQFKVTSDGANTLPDGVSLPGAYSATDPGILFNMYGSFDSYPIPGPSVWDGTSSGSSSSSSSSSSSSSAAAAVVATSSSSSSASIEAVTTKGAVAAVSTAAAVAPTTTTAAPTTFATAVASTKKATACRNKTKSSSAATTAAAVAETTSSTAAATAAASSASSASGTAGKYERCGGQGWTGATTCVDGWTCKQWNPYYYQCVESA

SEQ ID NO: 36 - protein, 406 aa, Aspergillus aculeatus
MRQAQSLSLLTALLSATRVAGHGHVTNVVVNGVYYEGFDINSFPYESDPPKVAAWTTPNTGNGFISPSDYGTDDIICHQNATNAQAHIVVAAGDKINIQWTAWPDSHHGPVLDYLARCDGECETVDKTTLEFFKIDGVGLISDTEVPGTWGDDQLIANNNSWLVEIPPTIAPGNYVLRHELIALHSAGTEDGAQNYPQCFNLQVTGSGTDEPAGTLGTKLYTEDEAGIVVNIYTSLSSYAVPGPTQYSGAVSVSQSTSAITSTGTAVVGSGSAVATSAAAATTSAAASSAAAATTAAAVTSANANTQIAQPSSSSSYSQIAVQVPSSWTTLVTVTPPAAAATTPAAVPEPQTPSASSGATTTSSSSGAAQSLYGQCGGINWTGATSCVEGATCYQYNPYYYQCISA

SEQ ID NO: 37 - protein, 427 aa, Aspergillus aculeatus
MSLSKIATLLLGSVSLVAGHGYVSSIEVDGTTYGGYLVDTYYYESDPPELIAWSTNATDDGYVSPSDYESVNIICHKGSAPGALSAPVAPGGWVQMTWNTWPTDHHGPVITYMANCHGSCADVDKTTLEFFKIDAGGLIDDTDVPGTWATDELIEDSYSRNITIPSDIAPGYYVLRHEIIALHSAENLDGAQNYPQCINLEVTGSETATPSGTLGTALYKETDPGIYVDIWNTLSTYTIPGPALYTAGSTATAAAAADTTTTSAGTTAEATTAAAAVSTTADAVPTESSAPSETSATTANPARPTAGSDIRFQPGQVKAGASVNNSATETSSGESATTTTTSVATAASSADSSTTSGVLSGACSQEGYWYCNGGTAFQRCVNGEWDASQSVAAGTVCTAGISETITISAAATRRDAMRRHLARPKRH

SEQ ID NO: 38 - protein, 267 aa, Aspergillus aculeatus
MLVKLISFLSAATSVAAHGHVSNIVINGVSYRGWDINSDPYNSNPPVVVAWQTPNTANGFISPDAYDTDDVICHLSATNARGHAVVAAGDKISLQWTTWPDSHHGPVISYLANCGSSCETVDKTTLEFFKIDGVGLVDESNPPGIWGDDELIANNNSWLVEIPASIAPGYYVLRHELIALHGAGSENGAQNYMQCFNLQVTGTGTVQPSGVLGTELYKPTDAGILVNIYQSLSTYVVPGPTLIPQAVSLVQSSSTITASGTAVTTTA

SEQ ID NO: 39 - protein, 273 aa, Aspergillus aculeatus
MKYLAIFAAAAAGLARPTAAHYIFSKLILDGEVSEDWQYIRKTTRETCYLPTKFTDTFDNLTPNDQDFRCNLGSFSNAAKTEVAEVEAGSTIGMQLFAGSHMRHPGPAQVFMSKAPSGNVQSYEGDGSWFKIWERTLCDKSGDLTGDAWCTYGQTEIEFQIPEATPTGEYLVRAEHIGLHRAQSNQAEFYYSCAQVKVTGNGTGVPSQTYQIPGMYNDRSELFNGLNLWSYSVENVEAAMKNSIVGDEIWNGSSVPSESHVPKYKKSHACRVY

SEQ ID NO: 40 - protein, 322 aa, Aurantiporus alborubescens
MRTIATFVTLVASVLPAVLAHGGVLSYSNGGNWYWGWKPYNSPDGQTTIQRPWATYNPITDATDPTIACNNDGTSGALQLTATVAAGSAITAYWNQVWPHDKGPMTTYLAQCPGSTCTGVNAKTLKWFKIDHAGLLSGTVYSGSWASGKMIAQNSTWTTTIPATVPSGNYLIRFETIALHSLPAQFYPECAQIQITGGGSRAPTAAELVSFPGAYSNNDPGVNIDIYSNAAQSATTYVIPGPPLYGGASGSGPSSAPPSSTPGSSSTSHGPTSVSTSSSAAPSTTGTVTQYGQCGGIGWAGATGCISPFKCTVINDYYYQCL

SEQ ID NO: 41 - protein, 234 aa, Aurantiporus alborubescens
MKAILAIFSALAPLAAAHYTFPDFIVNGTTTADWVYIRETANHYSNGPVTNVNDPEFRCYELDLQNTAASTLTATVSAGSSVGFKANSALYHPGYLDVYMSKATPAANSPSAGTDQSWFKVYESAPVFANGALSFPSENIQSFTFTIPKSLPSGQYLIRVEHIALHSASSYGGAQFYISCAQVNVVNGGNGNPGPLVKIPGVYTGNEPGILINIYSFPPGFSGYQSPGPAVWRG

SEQ ID NO: 42 - protein, 233 aa, Trichophaea saccata
MTPLKLRPLLLLVLSTTLSLVHAHYRFYELIANGATHASFEYIRQWVPIYSNSPVTDVTSVNLRCNVNATPAAEVITVAAGSTVGFVADTTVTHPGAFTAYMAKAPEDITEWDGNGDWFKIWEKGPTSITSSGITWDVTDTQWTFTIPSATPNGQYLLRFEHIALHAASTVGGAQFYMSCAQIQVTNGGNGSPGPTIKFPGGYSATDPGILINIYYPIPTSYTIPGPPVWTGK

SEQ ID NO: 43 - protein, 237 aa, Trichophaea saccata
MKCLLSLLLAATAVSAHTIFQEIGINGVMQARYDYMRLPSYDGPITDVTSTYMACNGGPNPLVQISNDVAFVKAGDSITLQWAQTLTTDFNTGLIIDPSHLGPVMVYMAKVPSATGPIPNSGWFKIYEDGYDPTTKTWAVTKLINNKGKVTVTIPSCLPAGDYLLRGEIIALHAASTYPGAQFYMECAQLRLTSGGTKMPTTYNIPGIYSPTDPGVTFNLYNGFTSYTIPGPRPFTC

SEQ ID NO: 44 - protein, 484 aa, Penicillium thomii
MSLSKISGLILGSAALVAGHGYVSGIVVDDTYYGGYLVTQYPYESDAPELIAWSEQETDLGYIDGSEYANSNIICHKEAKPGALEAPVKAGGSVELQWTTWPTSHHGPVITYMANCNGDCDDVDKTTLQFFKIDQGGLISDTTEPGTWATDNLIANNNSRTVTVPSDIADGNYVLRHEIIALHSAGETNGAQNYPQCINLKVTGGGSATPSGTLGTALYKNTDPGILINIYTSLSTYDIPGPTLYTAGAAAATAASTAASSTAAAVTTADAVTTAAAVTSSSASVEVVPTTTPSSSIVSAFPTWSPSSTPPFSNSSNGWRPSFSRGPGGPRFTSAPAPQFSAPSGAQQKQSATATPIVATPVVITMTETSTSWVTEMVTLTDKSVVQTTSAVPVVVAATTTLTEGSEPAQTASPSVVSGSSSSGSSSSSTTTTSKTSTGSDYVSSDWMSYLSSLSAAEVLQMLRQTFRWMVSNDKVHARDITIN

SEQ ID NO: 45 - protein, 320 aa, Talaromyces stipitatus
MPSTKVAALSAVLALASTVAGHGFVQNIVIDGKSYTGYLVNQYPYQSNPPAVIGWSTTATDLGFVDGSGYTNPDIICHKNAKPGQLSAPVAAGGKVELEWTTWPESHHGPVISYLANCNGDCTTVDKTKLEFVKIDQRGLIDDSNPPGTWAADQLIAANNSWTVTIPESIAPGNYVLRHEIIALHSANNATGAQNYPQCINLQITGSGTANPSGTPGEKLYTPTDPGILVNIYQSLSSYVIPGPTLWSGAAAHVVATAAGSATGVASATATPTTLVTAVSSPTGAPSVVTPEAPSVTSFAPVVTVTDVVTVTTVITTTIS

SEQ ID NO: 46 - protein, 272 aa, Thermomyces lanuginosus
MKGSSAASVLLTFLAGISRTSAHGYVSNLVINGVYYRGWLPGEDPYNPDPPIGVGWETPNLGNGFVTPSEASTDAVICHKEATPARGHVSVKAGDKIYIQWQPNPWPDSHHGPVLDYLAPCNGPCESVDKTSLRFFKIDGVGLIDGSSPPGYWADDELIANGNGWLVQIPEDIKPGNYVLRHEIIALHSAGNPDGAQLYPQCFNLEITGSGTVEPEGVPATEFYSPDDPGILVNIYEPLSTYEVPGPSLIPQAVQIEQSSSAITATGTPTPA

SEQ ID NO: 47 - protein, 327 aa, Thermomyces lanuginosus
MAFSTVTVFVTFLAFISIASAHGFVTKITVLGDNNKDYPGFDPSTPKEVPPGLDVAWSTSASDQGYMSSSNASYHSKDFICHRNAKPAPDAAQVHAGDKVQLHWTQWPGPEDHQGPILDYLASCNGPCSNVEKASLKWTKIDEAGRFPNGTWATDLLRNGGNTWNVTIPSDLAPGEYVLRNEIIALHSARNMGGAQHYMQCVNLNVTGTGHRELQGVSAAEFYNPTDPGILINVWQTQSLSSYHIPGPTLLAADTGNDGGHSASSTLATVTSRRLSTPSDAMPGNGSYGAISPPLKPAKGFHPVCNARFRHGSTFTLTTLVAPPART

SEQ ID NO: 48 - protein, 274 aa, Thermomyces lanuginosus
MKGSTTASLLLPLLASVTRTSAHGFVSNLVINGVFYRGWLPTEDPYKADPPIGVGWETPNLGNGFVLPEEASTDAIVCHKEAEPARGYASVAAGDKIYIQWQPNPWPESHHGPVIDYLAPCNGDCSTVNKTSLEFFKIDGVGLIDGSSPPGKWADDELIANGNGWLVQIPEDIKPGNYVLRHEIIALHEAFNQNGAQIYPQCFNLQITGSGTVEPEGTPATELYSPTDPGILVDIYNPLSTYVVPGPTLIPQAVEIEQSSSAVTATGTPTPAAA

SEQ ID NO: 49 - protein, 227 aa, Humicola insolens
MKLSVVLTGLAAALAEAHYTFPSVGNTADWQVVRQTTNFQSNGPVTDVNSDQIRCYERFPGQGAPGIYNVTAGQTISYNAKASISHPGPMAFYIAKVPAGYTAANWDGRGAVWSKIYQDMPRIAGSLTWPTNGARSVSVTIPRCLQDGHYLLRAEHIGLHSASGVGGAQFYISCAQLYVSGGTGTWNPRNKVAFPGAYSPTHPGIMINIYWPVPTSYTPPGPPVETC

SEQ ID NO: 50 - protein, 257 aa, Humicola insolens
MRPFLAALAAATTVHAHGWVDNATIDGVFYQLYHPYMDPYMGEFAPPRISRKLVWNGYVNDVTSIDLQCGGHTAEGQIGTEPAPLHAPATAGSTVNLRWTLWPDSHMGPIMTYMARCPDEGCDKWLPVWFKIHEAGRYTTDKSYPDDIWEVTRLMYPANEGYNYTIPACLASGHYLVRHEIIALHSAWAKGEAQFYPSCHQLTVTSIGGNVREAPAEYRVSFPGAYKDDDPGIFINVWNPGPYTIPGPPVWTCPESE

SEQ ID NO: 51 - protein, 246 aa, Humicola insolens
MRLSLTTLLASALSVQGHAIFQRVTVNGQDQGSLTGLRAPNNNNPVQNVNSQDIICGAPGSRSQSVINVNAGDRIGAWYQHVIGGAQFPGDPDNPIARSHKGPISVYLAKVDNAATANHQGLQWFKIWHDGFNPSTRQWAVDTMINNNGWVYFNLPQCIAPGHYLMRVELLALHSATYQGQAQFYISCAQINVQSGGNFTPWQTVSFPGAYQANHPGIQVNIYGAMGQPDNGGRPYQIPGPEPIQC

SEQ ID NO: 52 - protein, 265 aa, Humicola insolens
MGPTWAVILGLIAPSVLNIHGILLVNGTETPEWKYVLDVAPAVPISNPDSLPPGYQGYKVDPIIGSGNPNITCGRLAFDSAPKTQIADVLAGSEVGFRVSADGLGNRDLEKGYIPTFWHPGPAQAYLSRAPNDDLYSYKGDGDWFKIAYAGPVDDLTWSLWPGVSDFNFTIPLSTPPGKYLLRIENFMPTASTGYLQFYVNCAFVNIIGPGGGTPTEFIRIPGDYTDEDPGFLVPPEQSSLDGRVPRDQLKLMSYTPPGPAVWTG

SEQ ID NO: 53 - protein, 310 aa, Humicola insolens
MKALTLLAAATAASAHTIFVQLEADGTRYPVSHGVRTPQYDGPITDVSSNDLACNGGPNPTMKTDKIITVTAGSTVKAIWRHTLQSGPNDVMDPSHKGPTLAYLKKVDNALTDSGVGGGWFKIQEDGHSNGNWGTLKVINNQGIHYIDIPDCIDSGQYLLRAEMIALHAAGSPGGAQLYMECAQIEIVGGKGTVKPQTYSIPGIYKSNDPGILINIYSMSPSSQYIIPGPPLFTCNGGGGSNNGGGNNGGSNPPVQQPPATTLTTAIAQPTPICSVQQWGQCGGQGYSGCTTCASPYRCNEINAWYSQCL

SEQ ID NO: 54 - protein, 354 aa, Humicola insolens
MAPKTSTFLASLTGAALVAAHGHVSHIIVNGVQYRNYDPTTDFYSGNPPTVIGWSALNQDNGFIEPNNFGTPDIICHKSAKPGGGHVTVRAGDKISIVWTPEWPESHVGPVIDYLAACNGDCETVDKTSLRFFKIDGAGYDAAAGRWAADALRANGNSWLVQIPADLKAGNYVLRHEIIALHGAANPNGAQAYPQCINIRVTGGGNNQPSGVPGTQLYKASDPGILFNPWVANPQYPVPGPALIPGAVSSIPQSRSTATATGTATRPGADTDPTGVPPVVTTTSAPAQVTTTTSSRTTSLPQITTTFATSTTPPPPAATQSKWGQCGGNGWTGPTVCAPGSSCNKLNDWYSQCI

SEQ ID NO: 55 - protein, 267 aa, Humicola insolens
MYLLPIAAAALAFTTTAYAHAQVYGLRVNDQHQGDGRNKYIRSPSSNSPIRWDHVTHPFLICNIRDDNQPPGPAPDFVRAFAGDRVAFQWYHARPNDPTDYVLDSSHLGVLVTWIAPYTDGPGTGPIWTKIHQDGWNGTHWATSRLISNGGFVEFRLPGSLKPGKYLVRQEIIALHQADMPGPNRGPEFYPSCAQLEVFGSGEAAPPQGYDINKGYAESGDKLWFNIYINKNDEFKMPGPEVWDGGCRFGERWATEEPGKPKVNQHG

SEQ ID NO: 56 - protein, 237 aa, Humicola insolens
MKLLAPLMLAGAASAHTIFTSLEVDGRNYGTGNGVRVPSYNGPVEDVTSNSIACNGPPNPTSPTDTVITVQAGQNVTAIWRYMLNTQGTSPNDIMDSSHKGPTLAYLKKVNDARTDSGVGDGWFKIQHDGFDGTTWGTERVIFGQGRHTIKIPECIEPGQYLLRAEMIALHGAQNYPGAQFYMECAQLNIVGGTGTKKPSTVSFPGAYKGTDPGVKLSIWWPPVTNYVIPGPDVFKC

SEQ ID NO: 57 - protein, 234 aa, Humicola insolens
MKLLSTLAAIAATLATADAHYIFNILYVNGQRMGGEYTYVRRNSNSYFPVFPDILNSNDMRCNVGARPGNTQTATVRAGDRIGFKVFNNEVIEHPGPGFIYMSKAPGSVNNYDGSGDWFKVYETGLCRGGGNVDTNWCSYYKDRLEFTIPPKTPPGEYLVRIEHIGLHEGHVNRAQFYITCAQLKIEGPGGGNPNPLVKIPGIYRANDPGIAYNKWTNNPAPYIMPGPKVWDGN

SEQ ID NO: 58 - protein, 226 aa, Humicola insolens
MLGSALLLLGTALGATAHYTFPRINSGGDWQYVRRADNWQDNGFVGNVNSPQIRCFQSRHQAAPATLNVTAGSTVTYYANPNVYHPGPMAFYMARVPDGQDINSWTGEGAVWFKIYHEQPTGLGQQLRWSSDGKNSFQVQIPRCIRSGYYLLRAEHIGLHSAGSPGGAQFYISCAQLAVNGGGSTEPPNKVSFPGAYSPSDPGIQINIYWPVPTSYKNPGPPVFQC

SEQ ID NO: 59 - protein, 231 aa, Humicola insolens
MKLLPGLLLAATAAQAHYTFPRLVVNGQPEERDWSVTRMTKNHQSKSGIENPTSPDIRCYSSQTAPNVAIVPAGSTIHYISTQQINHPGPTQYYLAKVPAGQSAKTWDGSGNVWFKIATSMPEYDQNRQLVWPGHNTYQTINATIPANTPSGEYLLRVEQIALHMASQPNKAQFYISCSQIQITNGGNGTPGPLVAFPGAYRSNDPGILVNLYSGMQPSQYQPPGPAVWRG

SEQ ID NO: 60 - protein, 248 aa, Humicola insolens
MLLNSVIGSAVLLATGAAAHGAVTSYVIAGKNYPGYNGYAPSTTPNTIQWQWSTYDPIYSATDPKLRCNGGRSATQSAPAAPGDNITAIWQQWTHSQGPILVWMYKCPGAFSSCDGSGQGWFKIDEAGFNGDGKTVFLDTERPSGWEIAKLVGGNKGWTSTIPKNLAPGNYLVRHELIALHQANAPQWYPECAQVVITGSGTKEPPASYKAAIPGYCNQNDPNIRVPINDHSIPQTYKIPGPPVWRGE

SEQ ID NO: 61 - protein, 233 aa, Humicola insolens
MKLTTSIALLAAAGAQAHYTFPRTKVDGVTSGEWETIRITENHWSHGPVTDVTSQAMTCYEKTPGQGAPKTVNVKAGGTVTFTVDTDVGHPGPLHFYLAKVPAGKTAATFDGKGAVWFKIYQDGPGGLGTSSLTWPSFGKKEVSVQIPPCVQDGEYLLRVEHIALHSAASVGGAQLYISCAQINVTGGTGTLNPGQLVSFPGAYKPTDPGILFQLYWPPPTQYINPGPAPVKC

SEQ ID NO: 62 - protein, 243 aa, Humicola insolens
MKTLASALIAAGLLAQYAAAHAIFQFASSGGTDFGTSCVRMPPNNSPVTSVTSSDMACNVGGSRGVSGICEVNAGSDFTVEMHAQPNDRSCASEAIGGNHFGPVMVYMAKVDDATRADGASASWFKVDEFGYDAGSKTWGTDMLNKNCGKRTFRIPSKIPSGDYLVRAEAIALHTAGQPSGAQFYMSCYQVRIKGSNNGQLPAGVRIPGAYSATDPGILVDIWGNGFSQYTIPGPRVIDGSFF

SEQ ID NO: 63 - protein, 363 aa, Humicola insolens
MPRFTKSIVSALAGASLVAAHGHVTHIVINGVLYPNFDPTSHPYLQNPPTVVGWTAANTDNGFVAPDQFASGDIICHNQATNAGGHAVVAAGDKIWIQWDQWPESHHGPVLDYLASCGSSGCESVNKLDLEFFKIGEKGLIDGSSAPGRWASDELIANNAGWLVQIPADIAPGHYVLRHEIIALHAAGQPNGAQNYPQCFNLLVTGSGTARPQGVKGTALYTPNDKGILAGIYNAPVSYEIPGPALYSGAARNLQQSSSQATSTATALTGDAVPVPTQAPVTTTSSSSADAATATSTTVQPPQQTTLTTAIATSTAAAAPTTTAGSGNGGNRPFPTRCPGLAGLGFDKRRRQLRAEEGVQVVA

SEQ ID NO: 64 - protein, 296 aa, Humicola insolens
MKGLLSIAALSLAVGEASAHYIFQQLSTGGTKHPMWKYIRQHTNYNSPVIDLDSNDLRCNVGARGAGTETVTVAAGSSLTFHLDTPVYHQGPVSVYMSKAPGSVSDYDGSGGWFKIQDWGPTFTGSGATWKLDDSYTFNIPSCIPDGEYLVRIQSLGIHNPWPAGIPQFYISCAQVRVTGGGNANPSPQVSIPGAFKETDPGYTANIYNNFRSYTVPGPSVFTCSGNSGGGSNPSNPNPPTPTTFTTQVTTPTPASPPSCTVAKWGQCGGQGYSGCTNCEAGSTCRQQNAYYSQCI

SEQ ID NO: 65 - protein, 318 aa, Humicola insolens
MRPFSLVALATAVSGHAIFQRVSVNGVDQGQLKGVRAPSSNYPIENVNHPDFACNTNIQHRDGTVIKIPAGATVGAWWQHEIGGPSFPGDPDNPIAASHKGPIQVYLAKVDNAATASPNGLRWFKIAEKGLSGGVWAVDEMIRNNGWHYFTMPQCIAPGHYLMRVELLALHSASFPGGAQFYMECAQIEVTGSGNFSPSETVSFPGAYPANHPGIVVSIYDAQGNANNGGREYQIPGPRPITCSGGGSNNGGGNNNGGGNNNGGGNNNGGGNNNGGGNTGGGSAPLWGQCGGNGYTGPTTCAEGTCKKQNDWYSQCTP

SEQ ID NO: 66 - protein, 259 aa, Humicola insolens
MVLRSLSILAFVARGVFAHGGLSNYTVGDTWYSGYDPFTPAAAQLSQPWLIQRQWTSIDPLFSPTSPYLACNFPGTAPPSYIPLRAGDILTAVYWFWLHPVGPMSVWLARCAGDCRDEDVTRARWFKIWHAGFLEGPNLELGMWYQKKFQRWDGGPALWRVRIPRGLKKGLYMVRHEILSIHVGGRPQFYPECAHLNVTEGGEVVVPGEWTRRFPGAYDDDDKSVFIDIYRPEHENRTDYEIPGGPIWESLGEMELWPE

SEQ ID NO: 67 - protein, 325 aa, Humicola insolens
MRTVFAAALAALAAREVAGHATFQQLWVDGTDYGSTCVRLPASNSPLTDVTSSDFACNIGGRRGVGGKCPVKAGGVVTIEMHQQPNDRNCRSEAIGGMHWGPVQVYLSKVPDASTAEPTQVGWFKIFSNAWAKKPGGNSGDDDYWGTRELNGCCGRMDVPIPTDLEDGDYLLRAEALALHAMPGQFYMSCYQITITGGTGTAKPATVRFPGAYTNNDAGIRANIHAPLSTYIAPGPEVYSGGTTRAPGEGCPGCATTCQVGSSPSAQAPGHGTAVGGGAGGPSACTVQAYGQCGGQGYTGCTECADGFVCRDVSAPWYSQCQPAF

SEQ ID NO: 68 - protein, 298 aa, Humicola insolens
MRLPQVASVLALAAQVHGHGYIYRVTADNIVYPGYDIYVDPLLQPPPYRIAYGGGQTGPVYDINSKDIACQRVHSPAPGLIAQARAGSNITFWWSRWLYSHKGPISAWMAPYEGDIANVDVNQLEFFKIGEEFHDETGKWATEKLVDDPEGKWTVKIPADIKPGLYVVRNEIIALHFAVRMPPFFAAFTPLGPQFYMTCFAFNITGDGTATPQGYKFPGAYSKDDPALWWDLEENKNPYPGAGPKPHVSAYDVDLVPNELYIVSPTNNATADELYWEAQRQALAAQAATTEYFDSIGG

SEQ ID NO: 69 - protein, 298 aa, Humicola insolens
MHVQSLLAGALALAPSASAHFLFPHLMLNGVRTGAYEYVREHDFGFMPHNNDWINSPDFRCNEGSWRHRREPKTAVVTAGVDVVGFNLHLDFDLYHPGPVTIYLSRAPGDVRDYDGSGDWFKVYQLGTRQPFNGTDEGWATWKMKNWQFRLPAEIPAGEYLMRIEQMSVHPPYRQKEWYVQCAHLKINSNYNGPAPGPTIKIPGGYKISDPAIQYDQWAQPPPTYAPMPGPPLWPNNNPQQGNPNQGGNNGGGNQGGGNGGCTVPKWGQCGGQGYSGCRNCESGSTCRAQNDWYSQCL

SEQ ID NO: 70 - protein, 344 aa, Humicola insolens
MPPPLLATVLSLLALTRGALSHSHLAHVIINGQLYHGFDPRPNQNNHPARVGWSTTATDDGFVTPGNYSHPDIICHRGGVSPRAHAPVTAGGKVQVQWNGWPIGHVGPILTYIAPCGGLPGAEEGCTGVDKTDLRWTKIDDSMPPFRFTDATKPVSGRAQFPIGQVWATDALVEANNSWSVVIPRNIPPGPYVLRQEIVALHYAAKLNGAQNYPLCLNLWVEKGQQDQGEPFKFDAYDAREFYSEDHPGVLIDVMTMVGPRAVYRIPGPTVASGATRIPHSLQTSAETWVEGTPVAVTRATETVQMEITTTPAGQGAGVRTATPAMPTPTVTKRWKGRFEMGRP

SEQ ID NO: 71 - protein, 330 aa, Humicola insolens
MKSLTYAALAALWAQQTAAHATFQQLWVDGVDYGSQCARLPPSNSPIASVTSTAMRCNNGPRAAAKCPVKAGGTVTIEMHQQPGDRSCNQDAIGGAHHGPVMVYMSKVSDAFTADGSSGWFKIFQDGWAKNPNGRVGDDDFWGTKDLNTCCGKMNVKIPADIAPGDYLLRAEAIALHAAGPSGGAQPYVTCYQLTVTGGGNANPPTVNFPGAYSERDPGIAVSIHGALSNYVVPGPPVYSGGSEKRAGSPCEGCEATCKVGSSPSQTLAPSNPAPTSPANGGGNNGGGNTGGGCTVPKWQQCGGQGYSGCTVCESGSTCRAQNQWYSQCV

SEQ ID NO: 72 - protein, 216 aa, Humicola insolens
MKLLLPALLALAAESVSAHYIFQQLTVAGTKYPVWKYIRRNSNPAWLQNGPVTDLASTDLRCNVGGQVSNGTETLTVRAGDQFTFHLDTAVYHQGPTSLYMSRAPGKVEDYDGSGPWFKIYDWGPTGNNWVMRDSYTYNIPRCIPDGEYLLRIQQLGLHNPGAAPQFYISCAQIKVTGGGTTNPTPTALIPGAFRATDPGYTVNVSQTLSNSISTS

SEQ ID NO: 73 - protein, 490 aa, Humicola insolens
MRSVSLLAAAFAPLATAHTVFTALFINNVHQGDGTCVRMAKQGNLATHPVSLNSNEMACGRDGQQPVAFTCPAPAGAKLTLLFRMWADGSQPGSIDKSHVGPMSIYLKKVSDMNTDSAAGPGWFKIWSEGYDAATKKWATEKLIANNGLLSVNLPPGLPAGYYLARHEIVTLQNVTNNKADPQFYVGCAQLFVQGLGTAASVPADKTVSIPGHLNPNDPALVFNPYTQNAATYPSFGPPLFFPNAASAGSNKAQSTLKQTSGVIPSDCLIKNANWCGREVPDYTNEAGCWTAAGNCWEQADQCYKTAPPSGHKGCKTWEEQKCNVIQNSCEAKRFSGPPNRGVKFADMDVNQLVPGAIPEAVNAGQNGEAVVVDGTTSSADEKASVDLTTSSLPTPTPAAEENGKEDERLALDPTLTEDESFFSVEPTSEPTGVQVEVPLTTVVLLPTLTSSLNPLPTPTSISQPAHPGRPCTGRRRRPRPGFPKHPRDF

SEQ ID NO: 74 - protein, 306 aa, Humicola insolens
MFFRNAATLALAYATTGVSAHALMYGVWVNGVDQGDGRNVYIRTPPNNSPVKDLASPDIVCNVNGGRAVPDFVQASAGDTLTFEWLHNTRGDDIIDRSHLGPIITYIAPFTTGNPTGPVWTKIAEQGFNPSTRRWAVDDLIDNGGKTDFVLPASLAPGRYIIRQEIIAHHESETTFESNPARGAQFYPSCVQIQVSSGSGTAVPDQNFDFNTGYTYADPGIHFNIYTSFNSYSIPGPEVWTGASTGGGNGNGNGNGNATPTQPTPTPTVTPTPIETAQPVTTTTTSTRPFPTRCPGRRLKREEPKA

SEQ ID NO: 75 - protein, 339 aa, Humicola insolens
MAHPWARCVYTAIWLAASASGHSRVWSVSVNGRYQGPGVDDYLRAPPSDSPVVDLDSPTLNCNVNGNKPVPGFVEVSAGDSLEWKWYYINPYNPSDMIIAAEHRGPIITYITNYTDGQPQGAVWTKIDHEGYDPVTDRFAVDNLIANRGWKAIKLPMLADGKYILRQEIIALHSAHNQGGAQLYPNCIQIKVVGGKGSAVPNQNFDLNKGYTSDHPGLRFNLWQPFNNYTIPGPEVWKGVVVASNGTTNSTTNLTNNTGTGFANSTMATGETRTERSFMTLTASHSDTGVPAKSHTVAVSWTTSAAVVGSPISVTTTFSSFTTTPVPTNSTGAYLYRYK

SEQ ID NO: 76 - protein, 334 aa, Malbranchea cinnamomea
MSPSFKSTAILGAVALAARVRAHGYVSGIVVDGAYHGGYIVDKYPYMPNPPDVVGWSTTATDLGFVAPDAFGDPDIICHRDGAPGAIHAKVNAGATIELQWNTWPESHHGPVIDYLANCNGDCSSVDKTSLKFFKISEAGLNDGSNAPGQWASDDLIANNNSWTVTIPKSIAPGNYVLRHEIIALHSAGNQNGAQNYPQCFNLEITSNGSDNPEGVLGTELYKADDPGILFNIYQPMDSYPIPGPALYTGGSSPSPNPPTSTQSPVPQPTQSPPSGSNPGNGNGDDDNDNGNETPSPSLPVEIPDDLTSRELLLVAQEIIARLLELQNQLVVSN

SEQ ID NO: 77 - protein, 366 aa, Talaromyces leycettanus
MHQHFRYTALLTALLSASTRVASHGHVSNIVINGVPYQGWDIDSMPYESDPPVVVAWETPNTSNGFITPDQYGTSDIICHLNATNAKGHAVVAAGDKISIQWTAWPSSHHGPVISYLANCGASCETVDKTTLQFFKIDNIGFIDDSSPPGIWAADQLEANNNTWLVEIPPTIAPGYYVLRNEIIALHGAENQDGAQNYPQCFNLQVTGSGTDKPAGVLGTQLYSPTDPGILVNIYTSLSTYIVPGPTPYSGWVSVVQSSSAITASGTPVTGTGGVSPTTAATTTSSSHSTTSTTTGPTVTSTSHTTTTTTPTTLRTTTTTAAGGGATQTVYGQCGGSGWTGATACAAGATCSTLNPYYAQCLPTGA

SEQ ID NO: 78 - protein, 364 aa, Chaetomium thermophilum
  misc_feature (174)..(176): Xaa can be any naturally occurring amino acid
MPSFASKTLISALAGAASVAAHGHVKNFVINGLSYQAYDPTVFPYMQNPPIVAGWTASNTDNGFVGPESYSSPDIICHKSATNAKGHAVIKAGDSVYIQWDTWPESHHGPVIDYLASCGSAGCETVDKTQLEFFKIAEAGLIDGSQAPGKWAADQLIAQNNSWLVTIPENIKPXXXGSYVLRHEIIALHSAGQTNGAQNYPVCINLEVTGGGSDVPSGVKGTELYKPTDPGILINIYQSLSNYTIPGPALMPGAKPVTQHTSAIIGSTTAITGTATAAPAAPTSTAAAITTSSANANPAPTTTRGNANPVPTTTLRTSTIAPQPTAAPIQTPTSSVGRPPRPTRCPGLDNFKRARRHARDLAAH

SEQ ID NO: 79 - protein, 344 aa, Trichoderma ressei
MIQKLSNLLVTALAVATGVVGHGHINDIVINGVWYQAYDPTTFPYESNPPIVVGWTAADLDNGFVSPDAYQNPDIICHKNATNAKGHASVKAGDTILFQWVPVPWPHPGPIVDYLANCNGDCETVDKTTLEFFKIDGVGLLSGGDPGTWASDVLISNNNTWVVKIPDNLAPGNYVLRHEIIALHSAGQANGAQNYPQCFNIAVSGSGSLQPSGVLGTDLYHATDPGVLINIYTSPLNYIIPGPTVVSGLPTSVAQGSSAATATASATVPGGGSGPTSRTTTTARTTQASSRPSSTPPATTSAPAGGPTQTLYGQCGGSGYSGPTRCAPPATCSTLNPYYAQCLN

SEQ ID NO: 80 - protein, 252 aa, Acrophialophora fusispora
MRIEAITGLVLASAGAVSAHGWVDVWAIGGKNYTGFNPTVAPWVPDQGTIAWPAWNTDTGPVYSKDVNTTDIICSINATNAKIYSDPIAAGNVINLHWTVWPDSHHGPILSYLAACNGDCAKADKTKLKWFKIAHAGQISLGTGGGQVGYWASDKLQDDNGTWPVTIPASIKPGNYVLRNEIIALHSAYDVGAAQLYPQCVNIKITGNGRVTPAGVVGTKLYKETDPGLHYNIYNDESKPVYQIPGPALCKC

SEQ ID NO: 81 - protein, 344 aa, Corynascus sepedonium
MSKTSALLAGLTGAALVAAHGHVSHIIVNGVYYENYDPTTHWYQPNPPTVIGWTAAQQDNGFIEPNNFGTSDIICHKSGSPGGGHATVAAGDKINIVWTPEWPDSHIGPVIDYLAACNGDCETVNKESLRFFKIDGAGYDKAAGRWAAETLRQNGNSWLVQIPSDLKAGNYVLRHEIIALHGAGSANGAQAYPQCINLRVTGGGSSVPSGVAGTSLYKASDAGILFNPYVASPDYPVPGPALIAGAASSIVQSTSAVTATASATAPGGGGANPNPTPTTTSSSNPAPSTTLRTTTSAAQTTPPPTNGNVQTKYGQCGGRDWSGPTACAAGSSCSVLNDWYSQCV

SEQ ID NO: 82 - protein, 347 aa, Corynascus sepedonium
MPSSTSKGLFSALMGAASVAAHGHVTNIVINGVSYQNYDPTSFPYMQNPPTVVGWTASNTDNGFVAPDAFASGDIICHRDATNAGGHAVVAAGDKVFIQWDTWPESHHGPVLDYLASCGDAGCETVDKNTLEFFKIGEAGLIDGSSAPGKWASDQLIENNNSWMVQIPANLAPGNYVLRHEIIALHSAGQANGAQNYPQCFNLQVTGSGTDKPAGVLGTELYTPTDAGILANIYTSPVQYEIPGPALISGASAVEQSSSAITASASAETGSATAPPAGSATAAPTTTTTTAGSDASATPSSSSSSGASTTAEPTPSATTTAGGSTPRPTRCPGLKRRRHARDVKLAL

SEQ ID NO: 83 - protein, 342 aa, Myceliophthora thermophila
MSKASALLAGLTGAALVAAHGHVSHIVVNGVYYRNYDPTTDWYQPNPPTVIGWTAADQDNGFVEPNSFGTPDIICHKSATPGGGHATVAAGDKINIVWTPEWPESHIGPVIDYLAACNGDCETVDKSSLRWFKIDGAGYDKAAGRWAADALRANGNSWLVQIPSDLKAGNYVLRHEIIALHGAQSPNGAQAYPQCINLRVTGGGSNLPSGVAGTSLYKATDPGILFNPYVSSPDYTVPGPALIAGAASSIAQSTSVATATGTATVPGGGGANPTATTTAATSAAPSTTLRTTTTSAAQTTAPPSGDVQTKYGQCGGNGWTGPTVCAPGSSCSVLNEWYSQCL

SEQ ID NO: 84 - protein, 254 aa, Talaromyces emersonii
MLSSKAPVTLAFAGLAGLLSAPLVKAHGFVQGIVIGDQFYSGYIVNEFPYESNPPPVIGWATTATDLGFVDGTEYQGPDIICHRNATPALLTAPVAAGGTVELQWTPWPSSHHGPVITYLANCNGNCSTVDKTQLEFFKIDQSGLINDTDPPGTWASDNLIANNNSWTVTIPSTLEPGNYVLRHEIIALHSAGNKDGAQNYPQCINIEVTGGGSVEPTGTLGEDLYHDTDPGILIDIYEPIATYTIPGPPEPTF

SEQ ID NO: 85 - protein, 272 aa, Talaromyces thermophilus
MKAPSAASILLPFLASITRTSAHGFVSNIVINGVSYRGWLPNEDPYKPEPPIGVGWETPNLSNGFVTPEEALTDAIVCHKEAKPARGYASVAAGDKIYIQWQPIPWPESHHGPVLDYLAPCNGDCQNVNKSSLEFFKIDGKGLIDGSSPPGFWADDELIANGNGWLVQIPEDIKPGNYVLRHEIIALHEGFNQNGAQLYPQCFNLQITGSGTVEPEGTPATELYSPTDPGILVDIYNPLSTYVVPGPTLIPQAVEIEQSSSAVTATGTPTPA

SEQ ID NO: 86 - protein, 272 aa, Talaromyces thermophilus
MKGSSAASVLLALLAGITRTSAHGYVSNIVVNGVYYRGWLPGEDPYNPDPPIGVGWETPNLGNGFVTPEEASTDAIICHKEAKPARGHATVKAGDKIYIQWQPIPWPESHHGPVLDYLAACNGDCETVDKTSLRFFKISNKGLIDGSSPPGYWADDQLIENGNGWLVQIPEDIKPGNYVLRHEIIALHAAGNPNGAQLYPQCFNLHITGSGTVEPQGIPATELYSPDDPGILINIYQPLTTYEVPGPTPIPQAVEIEQSSSAITATGTPTPA

SEQ ID NO: 87 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 88 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 89 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 90 - protein, 740 aa, Thermoascus aurantiacus
MRAIGLLPGIIGIAGAACPYMTGELPRSFAENPHAINRRAEGGGGAAAETEKFLSQFYLNDNDTFMTTDVGGPIEDQNSLSAGDRGPTLLEDFILRQKIQRFDHERVPERAVHARGAGAHGVFTSYADWSNITAASFLSAAGKETPVFVRFSTVAGSRGSADTARDVHGFATRFYTDEGNFDIVGNNIPVFFIQDAIQFPDLIHAVKPSPNNEIPQAATAHDSAWDFFSQQPSSLHTLFWAMAGHGIPRSYRNMDGFGIHTFRFVTDDGASKLVKFHWTSLQGKASLVWEEAQAVAGKNADYHRQDLWDAIEAGRYPEWELGVQIMDEEDQLRFGFDLLDPTKIVPEEYVPITKLGKMQLNRNPLNYFAETEQIMFQPGHVVRGIDFTEDPLLQGRLFSYLDTQLNRHGGPNFEQIPINRPRTPIHNNNRDGAAQMYIPLNKAAYTPNTLNNGSPKQANQTVGKGFFTTPGRTASGRLVRAVSSTFADVWSQPRLFYNSLVPAEQQFLINAIRFETAHITSDVVKNNVIIQLNRVSNNLAKRVARAIGVAEPEPDPTLYHNNKTANVGVFGKPLARLDGLQVGVLATVNKPDSIKQAASLKASFAADNVDVKVVAERLADGVDETYSAADAVNFDAILVANGAEGLFARDSFTARPANSTTATLYPAGRPLQILVDGFRYGKPVGALGSGAKALDAAEISTTRAGVYVANSTTDSFINGVRDGLRTFKFLDRFAIDEDAE

SEQ ID NO: 91 - protein, 620 aa, Myceliophthora thermophila
MKSFISAATLLVGILTPSVAAAPPSTPEQRDLLVPITEREEAAVKARQQSCNTPSNRACWTDGYDINTDYEVDSPDTGVVRPYTLTLTEVDNWTGPDGVVKEKVMLVNNSIIGPTIFADWGDTIQVTVINNLETNGTSIHWHGLHQKGTNLHDGANGITECPIPPKGGRKVYRFKAQQYGTSWYHSHFSAQYGNGVVGAIQINGPASLPYDTDLGVFPISDYYYSSADELVELTKNSGAPFSDNVLFNGTAKHPETGEGEYANVTLTPGRRHRLRLINTSVENHFQVSLVNHTMTIIAADMVPVNAMTVDSLFLGVGQRYDVVIEASRTPGNYWFNVTFGGGLLCGGSRNPYPAAIFHYAGAPGGPPTDEGKAPVDHNCLDLPNLKPVVARDVPLSGFAKRPDNTLDVTLDTTGTPLFVWKVNGSAINIDWGRPVVDYVLTQNTSFPPGYNIVEVNGADQWSYWLIENDPGAPFTLPHPMHLHGHDFYVLGRSPDESPASNERHVFDPARDAGLLSGANPVRRDVTMLPAFGWVVLAFRADNPGAWLFHCHIAWHVSGGVGVVYLERADDLRGAVSDADADDLDRLCADWRRYWPTNPYPKSDSGLKHRWVEEGEWLVKA

SEQ ID NO: 92 - protein, 520 aa, Polyporus pinsitus
MSRFHSLLAFVVASLTAVAHAGIGPVADLTITNAAVSPDGFSRQAVVVNGGTPGPLITGNMGDRFQLNVIDNLTNHTMVKSTSIHWHGFFQKGTNWADGPAFINQCPISSGHSFLYDFQVPDQAGTFWYHSHLSTQYCDGLRGPFVVYDPNDPAADLYDVDNDDTVITLVDWYHVAAKLGPAFPLGADATLINGKGRSPSTTTADLSVISVTPGKRYRFRLVSLSCDPNYTFSIDGHNMTIIETDSINTAPLVVDSIQIFAAQRYSFVLEANQAVDNYWIRANPNFGNVGFTGGINSAILRYDGAAAVEPTTTQTTSTAPLNEVNLHPLVTTAVPGSPVAGGVDLAINMAFNFNGTNFFINGTSFTPPTVPVLLQIISGAQNAQDLLPSGSVYSLPSNADIEISFPATAAAPGAPHPFHLHGHAFAVVRSAGSTVYNYDNPIFRDVVSTGTPAAGDNVTIRFRTDNPGPWFLHCHIDFHLEAGFAVVFAEDIPDVASANPVPQAWSDLCPTYDALDPSDQ

SEQ ID NO: 93 - protein, 363 aa, Glycine max
MKFFTTILSTASLVAALPAAVDSNHTPAAPELVARLGQLTPTFYRETCPNLFPIVFGVIFDASFTDPRIGASLMRLHFHDCFVQGCDGSVLLNNTDTIESEQDALPNINSIRGLDVVNDIKTAVENSCPDTVSCADILAIAAEIASVLGGGPGWPVPLGRRDSLTANRTLANQNLPAPFFNLTQLKASFAVQGLNTLDLVTLSGGHTFGRARCSTFINRLYNFSNTGNPDPTLNTTYLEVLRARCPQNATGDNLTNLDLSTPDQFDNRYYSNLLQLNGLLQSDQELFSTPGADTIPIVNSFSSNQNTFFSNFRVSMIKMGNIGVLTGDEGEIRLQCNFVNGDSFGLASVASKDAKQKLVAQSK

SEQ ID NO: 94 - protein, 363 aa, Coprinus cinereus
  misc_feature (293)..(295): Xaa can be any naturally occurring amino acid
MKLSLLSTFAAVIIGALALPQGPGGGGSVTCPGGQSTSNSQCCVWFDVLDDLQTNFYQGSKCESPVRKILRIVFHDAIGFSPALTAAGQFGGGGADGSIIAHSNIELAFPANGGLTDTVEALRAVGINHGVSFGDLIQFATAVGMSNCPGSPRLEFLTGRSNSSQPSPPSLIPGPGNTVTAILDRMGDAGFSPDEVVDLLAAHSLASQEGLNSAIFRSPLDSTPQVFDTQFYIETLLKGTTQPGPSLGFAEELSPFPGEFRMRSDALLARDSRTACRWQSMTSSNEVMGQRYXXXMAKMSVLGFDRNALTDCSDVIPSAVSNNAAPVIPGGLTVDDIEVSCPSEPFPEIATASGPLPSLAPAP

(19) United States
(12) Patent Application Publication
(10) Pub. No.: US 2016/0298154 A1
(43) Pub. Date: Oct. 13, 2016

(54) Processes for Increasing Enzymatic Hydrolysis of Cellulosic Material

(71) Applicant: Novozymes A/S, Bagsvaerd (DK)

(72) Inventors: Jiyin Liu, Raleigh, NC (US); Hui Xu, Wake Forest, NC (US); Feng Xu, Davis, CA (US); Ye Chen, Cary, NC (US); Terry Green, Wake Forest, NC (US)

(73) Assignee: Novozymes A/S, Bagsvaerd (DK)

(21) Appl. No.: 14/916,119

(22) PCT Filed: Sep. 4, 2014
(86) PCT No.: PCT/US2014/054067

§ 371 Date: Mar. 2, 2016

Related U.S. Application Data
(60) Provisional application No. 61/873,586, filed on Sep. 4, 2013.

(51) Int. Cl. C12P 19/14; C12P 19/02

(52) U.S. Cl. CPC C12P 19/14; C12P 19/02; C12Y 110/03002; C12Y 111/01006; C12Y 302/01004; C12P 2201/00; C12P 2203/00

(57) ABSTRACT

The present invention relates to methods for increasing hydrolysis of a cellulosic material, comprising: hydrolyzing the cellulosic material with an enzyme composition in the presence of a combination of an AA9 polypeptide and one or more oxidoreductases selected from the group consisting of a catalase, a laccase, and a peroxidase.

### REFERENCE TO A SEQUENCE LISTING

This application contains a Sequence Listing, which is incorporated herein by reference.

### BACKGROUND OF THE INVENTION

1. Field of the Invention

The present invention relates to methods for increasing hydrolysis of cellulosic material with an enzyme composition.

2. Description of the Related Art

Cellulose is a polymer of the simple sugar glucose linked by beta-1,4-bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

There is a need in the art to improve the performance of cellulose-hydrolyzing enzyme systems.

WO 2010/012579 discloses methods for the modification of a material comprising a non-starch carbohydrate, which method comprises contacting said material comprising a non-starch carbohydrate with a polypeptide having peroxidase activity. WO 2010/080408 discloses methods for increasing hydrolysis of cellulosic material with an enzyme composition in the presence of a peroxidase.

The present invention provides processes for increasing hydrolysis of cellulosic materials with enzyme compositions.

### SUMMARY OF THE INVENTION

The present invention relates to processes for degrading a cellulosic material, comprising: treating the cellulosic material with an enzyme composition in the presence of a combination of an AA9 polypeptide and one or more oxidoreductases selected from the group consisting of a catalase, a laccase, and a peroxidase.

The present invention also relates to processes for producing a fermentation product, comprising:

(a) saccharifying a cellulosic material with an enzyme composition in the presence of an enzyme composition in the presence of a combination of an AA9 polypeptide and one or more oxidoreductases selected from the group consisting of a catalase, a laccase, and a peroxidase;

(b) fermenting the saccharified cellulosic material with one or more (e.g., several) fermenting microorganisms to produce the fermentation product; and

(c) recovering the fermentation product from the fermentation.

The present invention also relates to processes of fermenting a cellulosic material, comprising: fermenting the cellulosic material with one or more fermenting microorganisms, wherein the cellulosic material is hydrolyzed with an enzyme composition in the presence of a combination of an AA9 polypeptide and one or more oxidoreductases selected from the group consisting of a catalase, a laccase, and a peroxidase.

The present invention further relates to enzyme compositions comprising a combination of an AA9 polypeptide and one or more oxidoreductases selected from the group consisting of a catalase, a laccase, and a peroxidase.

### BRIEF DESCRIPTION OF THE FIGURES

FIG. 1 shows synergy between Coprinus cinereus peroxidase and Thermoascus aurantiacus AA9 (GH61A) polypeptide in increasing the hydrolysis of pretreated corn stover (PCS) by a cellulase composition at pH 5 for 120 hours.

FIG. 2 shows synergy between Thermoascus aurantiacus catalase and T. aurantiacus AA9 GH61A) polypeptide in increasing the hydrolysis of pretreated corn stover (PCS) by a cellulase composition at pH 5 for 120 hours.

FIG. 3 shows synergy between Myceliophthora thermophila laccase and T. aurantiacus AA9 (GH61A) polypeptide in increasing the hydrolysis of pretreated corn stover (PCS) by a cellulase composition at pH 5 for 120 hours.

FIG. 4 shows synergy between T. aurantiacus catalase, M. thermophila laccase, and T. aurantiacus AA9 (GH61A) polypeptide, Penicillium sp. (emersonii) AA9 (GH61A) polypeptide, or Aspergillus fumigatus AA9 (GH61B) polypeptide variant in increasing the hydrolysis of pretreated corn stover (PCS) by a cellulase composition at pH 5 for 72 hours.

FIG. 5 shows synergy between T. aurantiacus catalase, M. thermophila laccase, and T. aurantiacus AA9 (GH61A) polypeptide, Penicillium sp. (emersonii) AA9 (GH61A) polypeptide, or A. fumigatus AA9 (GH61B) polypeptide variant in increasing the hydrolysis of pretreated corn stover (PCS) by a cellulase composition at pH 5 for 120 hours.

FIG. 6 shows synergy between T. aurantiacus catalase, M. thermophila laccase, and Thermomyces lanuginosus AA9 (GH61) polypeptide in increasing the hydrolysis of pretreated corn stover (PCS) by a cellulase composition at pH 5 for 72 hours.

FIG. 7 shows synergy between T. aurantiacus catalase, Myceliophthora thermophila laccase, and T. lanuginosus AA9 (GH61) polypeptide in increasing the hydrolysis of pretreated corn stover (PCS) by a cellulase composition at pH 5 for 120 hours.

FIG. 8 shows synergy between T. aurantiacus AA9 (GH61A) polypeptide and an individual oxidoreductase in the hydrolysis of pretreated corn stover (PCS) by a cellulase composition at pH 5 for 72 hours.

FIG. 9 shows synergy between T. aurantiacus AA9 (GH61A) polypeptide and an individual oxidoreductase in the hydrolysis of pretreated corn stover (PCS) by a cellulase composition at pH 5 for 120 hours.

FIG. 10 shows synergy between T. aurantiacus AA9 (GH61A) polypeptide and multiple oxidoreductases in the hydrolysis of pretreated corn stover (PCS) by a cellulase composition at pH 5 for 72 hours.

FIG. 11 shows synergy between T. aurantiacus AA9 (GH61A) polypeptide and multiple oxidoreductases in the hydrolysis of pretreated corn stover (PCS) by a cellulase composition at pH 5 for 120 hours.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity can be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity can be determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Auxiliary Activity 9 polypeptide: The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C. and a suitable pH, such as 4-9, e.g., 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5 L (Novozymes NS, Bagsvaerd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASO), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASO.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can be used in the presence of a soluble activating divalent metal cation according to WO 2008/151043 or WO 2012/122518, e.g., manganese or copper.

The AA9 polypeptide can also be used in the presence of a dioxy compound, a bicylic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1-4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Catalase: The term “catalase” means a hydrogen-peroxide:hydrogen-peroxide oxidoreductase (E.C. 1.11.1.6 or E.C. 1.11.1.21) that catalyzes the conversion of two hydrogen peroxides to oxygen and two waters.

Catalase activity can be determined by monitoring the degradation of hydrogen peroxide at 240 nm based on the following reaction:

2H₂O₂→2H₂O+O₂

The reaction is conducted in 50 mM phosphate pH 7 at 25° C. with 10.3 mM substrate (H₂O₂). Absorbance is monitored spectrophotometrically within 16-24 seconds, which should correspond to an absorbance reduction from 0.45 to 0.4. One catalase activity unit can be expressed as one μmole of H₂O₂ degraded per minute at pH 7.0 and 25° C.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pretreated corn stover (PCS) (or other pretreated cellulosic material) for 3-7 days at a suitable temperature such as 40° C.-80° C., e.g., 40° C., 45° C., 50° C., 55° C., 60° C., 65° C., 70° C., 75° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one aspect, the cellulosic material is any biomass material. In another aspect, the cellulosic material is lignocellulose, which comprises cellulose, hemicelluloses, and lignin.

In an embodiment, the cellulosic material is agricultural residue, herbaceous material (including energy crops), municipal solid waste, pulp and paper mill residue, waste paper, or wood (including forestry residue).

In another embodiment, the cellulosic material is arundo, bagasse, bamboo, corn cob, corn fiber, corn stover, miscanthus, rice straw, sugar cane straw, switchgrass, or wheat straw.

In another embodiment, the cellulosic material is aspen, eucalyptus, fir, pine, poplar, spruce, or willow.

In another embodiment, the cellulosic material is algal cellulose, bacterial cellulose, cotton linter, filter paper, microcrystalline cellulose (e.g., AVICEL®), or phosphoric-acid treated cellulose.

In another embodiment, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Dissolved Oxygen Saturation Level: The saturation level of oxygen is determined at the standard partial pressure (0.21 atmosphere) of oxygen. The saturation level at the standard partial pressure of oxygen is dependent on temperature and solute concentrations. In an embodiment where the temperature during hydrolysis is 50° C., the saturation level would typically be in the range of 5-5.5 mg oxygen per kg slurry, depending on the solute concentrations. Hence, dissolved oxygen is present in a range from 0.025 ppm to 0.55 ppm, such as, e.g., 0.05 to 0.165 ppm at temperatures around 50° C.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. Feruloyl esterase activity can be determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Fragment: The term “fragment” means a polypeptide having one or more (e.g., several) amino acids absent from the amino and/or carboxyl terminus of a mature polypeptide main; wherein the fragment has xylanase activity. In one aspect, a fragment contains at least 85% of the amino acid residues, e.g., at least 90% of the amino acid residues or at least 95% of the amino acid residues of a polypeptide having biological activity.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 40° C., 45° C., 50° C., 55° C., 60° C., 65° C., 70° C., 75° C., or 80° C., and a suitable pH such as 4-9, e.g., 4.0, 4.5, 5.0, 5.5, 6.0, 6.5, 7.0, 7.5, 8.0, 8.5, or 9.0.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Laccase: The term “laccase” means a benzenediol:oxygen oxidoreductase (E.C. 1.10.3.2) that catalyzes the following reaction: 1,2- or 1,4-benzenediol+O₂=1,2- or 1,4-benzosemiquinone+2H₂O.

Laccase activity can be determined by the oxidation of syringaldazine (4,4′-[azinobis(methanylylidene)]bis(2,6-dimethoxyphenol)) to the corresponding quinone 4,4′-[azobis(methanylylidene])bis(2,6-dimethoxycyclohexa-2,5-dien-1-one) by laccase. The reaction (shown below) is detected by an increase in absorbance at 530 nm.

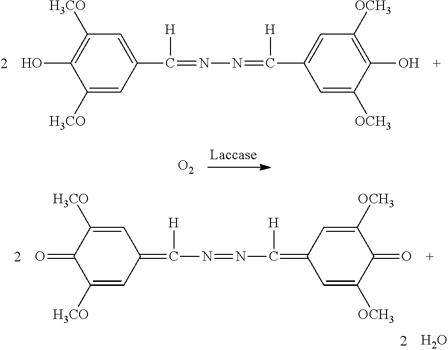 [SMILES: COc1cc(C=NN=Cc2cc(OC)c(O)c(OC)c2)cc(OC)c1O ; "O2" ; COC1=CC(=CN=NC=C2C=C(OC)C(=O)C(OC)=C2)C=C(OC)C1=O ; O]

The reaction is conducted in 23 mM MES pH 5.5 at 30° C. with 19 μM substrate (syringaldazine) and 1 g/L polyethylene glycol (PEG) 6000. The sample is placed in a spectrophotometer and the change in absorbance is measured at 530 nm every 15 seconds up to 90 seconds. One laccase unit is the amount of enzyme that catalyzes the conversion of 1 μmole syringaldazine per minute under the specified analytical conditions.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having xylanase activity.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Peroxidase: The term “peroxidase” means an enzyme that converts a peroxide, e.g., hydrogen peroxide, to a less oxidative species, e.g., water. It is understood herein that a peroxidase encompasses a peroxide-decomposing enzyme. The term “peroxide-decomposing enzyme” is defined herein as an donor:peroxide oxidoreductase (E.C. number 1.11.1.x, wherein x=1-3, 5, 7-19, or 21) that catalyzes the reaction reduced substrate(2e⁻)+ROOR′→oxidized substrate+ROH+R′OH; such as horseradish peroxidase that catalyzes the reaction phenol+H₂O₂→quinone+H₂O, and catalase that catalyzes the reaction H₂O₂+H₂O₂→O₂+2H₂O. In addition to hydrogen peroxide, other peroxides may also be decomposed by these enzymes.

Peroxidase activity can be determined by measuring the oxidation of 2,2′-azino-bis(3-ethylbenzthiazoline-6-sulfonic acid (ABTS) by a peroxidase in the presence of hydrogen peroxide as shown below. The reaction product ABTSₒₓ forms a blue-green color which can be quantified at 418 nm.

H₂O₂+2ABTS_{red}+2H⁺→2H₂O+2ABTSₒₓ

The reaction is conducted in 0.1 M phosphate pH 7 at 30° C. with 1.67 mM substrate (ABTS), 1.5 g/L TRITON® X-405, 0.88 mM hydrogen peroxide, and approximately 0.040 units enzyme per ml. The sample is placed in a spectrophotometer and the change in absorbance is measured at 418 nm from 15 seconds up to 60 seconds. One peroxidase unit can be expressed as the amount of enzyme required to catalyze the conversion of 1 μmole of hydrogen peroxide per minute under the specified analytical conditions.

Pretreated cellulosic or hemicellulosic material: The term “pretreated cellulosic or hemicellulosic material” means a cellulosic or hemicellulosic material derived from biomass by treatment with heat and dilute sulfuric acid, alkaline pretreatment, neutral pretreatment, or any pretreatment known in the art.

Pretreated corn stover: The term “Pretreated Corn Stover” or “PCS” means a cellulosic material derived from corn stover by treatment with heat and dilute sulfuric acid, alkaline pretreatment, neutral pretreatment, or any pretreatment known in the art.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0, 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having xylanase activity. In one aspect, a subsequence contains at least 85% of the nucleotides, e.g., at least 90% of the nucleotides or at least 95% of the nucleotides of a polynucleotide encoding a polypeptide having biological activity.

Variant: The term “variant” means a polypeptide having xylanase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrimann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al., 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylan degrading activity can be determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Reference to “about” a value or parameter herein includes aspects that are directed to that value or parameter per se. For example, description referring to “about X” includes the aspect “X”.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

Unless defined otherwise or clearly indicated by context, all technical and scientific terms used herein have the same meaning as commonly understood by one of ordinary skill in the art to which this invention belongs.

### DETAILED DESCRIPTION OF THE INVENTION

The present invention relates to processes for degrading a cellulosic material, comprising: treating the cellulosic material with an enzyme composition in the presence of a combination of an AA9 polypeptide and one or more (e.g., several) oxidoreductases selected from the group consisting of a catalase, a laccase, and a peroxidase. In one aspect, the processes further comprise recovering the degraded cellulosic material. Soluble products from the degradation of the cellulosic material can be separated from insoluble cellulosic material using methods known in the art such as, for example, centrifugation, filtration, or gravity settling.

The present invention also relates to processes of producing a fermentation product, comprising: (a) saccharifying a cellulosic material with an enzyme composition in the presence of a combination of an AA9 polypeptide and one or more (e.g., several) selected from the group consisting of a catalase, a laccase, and a peroxidase; (b) fermenting the saccharified cellulosic material with one or more (e.g., several) fermenting microorganisms to produce the fermentation product; and (c) recovering the fermentation product from the fermentation.

The present invention also relates to processes of fermenting a cellulosic material, comprising: fermenting the cellulosic material with one or more (e.g., several) fermenting microorganisms, wherein the cellulosic material is saccharified with an enzyme composition in the presence of a combination of an AA9 polypeptide and one or more (e.g., several) oxidoreductases selected from the group consisting of a catalase, a laccase, and a peroxidase. In one aspect, the fermenting of the cellulosic material produces a fermentation product. In another aspect, the processes further comprise recovering the fermentation product from the fermentation.

A synergistic effect between an AA9 polypeptide and one or more oxidoreductases is defined as an effect arising between the AA9 polypeptide and the one or more oxidoreductases that produces an effect greater than the sum of their individual effects. In each of the processes described above, the presence of the combination of the AA9 polypeptide and the one or more oxidoreductases synergistically increases the hydrolysis of the cellulosic material by the enzyme composition at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold, compared to the AA9 polypeptide alone, the one or more oxidoreductases alone, or absence of the AA9 polypeptide and the one or more oxidoreductases.

The present invention also relates to enzyme composition comprising a combination of an AA9 polypeptide and one or more oxidoreductases selected from the group consisting of a catalase, a laccase, and a peroxidase. The enzyme compositions may be prepared in accordance with methods known in the art and may be in the form of a liquid or a dry composition. The compositions may be stabilized in accordance with methods known in the art.

In one aspect, the one or more oxidoreductases are one oxidoreductase. In another aspect, the one or more oxidoreductases are two oxidoreductases. In another aspect, the one or more oxidoreductases are three oxidoreductases. In another aspect, the one or more oxidoreductases are at least one oxidoreductase. In another aspect, the one or more oxidoreductases are at least two oxidoreductases. In another aspect, the one or more oxidoreductases are at least three oxidoreductases.

In another aspect, the combination of the AA9 polypeptide and the one or more oxidoreductases is a combination of an AA9 polypeptide and a catalase; a combination of an AA9 polypeptide and a laccase; or a combination of an AA9 polypeptide and a peroxidase.

In another aspect, the combination of the AA9 polypeptide and the one or more oxidoreductases is a combination of an AA9 polypeptide, a catalase, and a laccase; a combination of an AA9 polypeptide, a catalase, and a peroxidase; a combination of an AA9 polypeptide, a laccase, and a peroxidase; or a combination of an AA9 polypeptide, a catalase, a laccase, and a peroxidase.

In another aspect, the combination of the AA9 polypeptide and the one or more oxidoreductases is a combination of an AA9 polypeptide and two catalases; a combination of an AA9 polypeptide and two laccases; or a combination of an AA9 polypeptide and two peroxidases.

In another aspect, the combination of the AA9 polypeptide and the one or more oxidoreductases is a combination of an AA9 polypeptide, a laccase, and two catalases; a combination of an AA9 polypeptide, a peroxidase, and two catalases; a combination of an AA9 polypeptide, a catalase, and two laccases; a combination of an AA9 polypeptide, a peroxidase, and two laccases; a combination of an AA9 polypeptide, a catalase, and two peroxidases; a combination of an AA9 polypeptide, a laccase, and two peroxidases; a combination of an AA9 polypeptide and three catalases; a combination of an AA9 polypeptide and three laccases; or a combination of an AA9 polypeptide and three peroxidases.

In an embodiment of the combination of an AA9 polypeptide and an oxidoreductase, the protein content of the AA9 polypeptide and the oxidoreductase is in the range of about 0.5% to about 25%, e.g., about 0.5% to about 20%, about 0.5% to about 15%, about 0.5% to about 10%, about 0.5% to about 7.5%, about 0.5% to about 5%, and about 0.5% to about 4% of total protein. The protein ratio of AA9 polypeptide to catalase is in the range of about 0.5:1 to about 15:1, e.g., about 0.8:1 to about 5:1 or about 2:1. The protein ratio of AA9 polypeptide to laccase is in the range of about 3:1 to about 150:1, e.g., about 5:1 to about 50:1 or about 10:1. The protein ratio of AA9 polypeptide to peroxidase is in the range of about 0.5:1 to about 15:1, e.g., about 0.8:1 to about 5:1 or about 2:1.

In another embodiment of the combination of an AA9 polypeptide and two oxidoreductases, the protein content of the AA9 polypeptide and the two oxidoreductases is in the range of about 0.5% to about 25%, e.g., about 0.5% to about 20%, about 0.5% to about 15%, about 0.5% to about 10%, about 0.5% to about 7.5%, about 0.5% to about 5%, and about 0.5% to about 4% of total protein. The protein ratio of AA9 polypeptide to catalase is in the range of about 1:1 to about 30:1, e.g., about 1.6:1 to about 10:1 or about 4:1. The protein ratio of AA9 polypeptide to laccase is in the range of about 6:1 to about 300:1, e.g., about 10:1 to about 100:1 or about 20:1. The protein ratio of AA9 polypeptide to peroxidase is in the range of about 1:1 to about 30:1, e.g., about 1.6:1 to about 10:1 or about 4:1.

In another embodiment of the combination of an AA9 polypeptide and three oxidoreductases, the protein content of the AA9 polypeptide and the three oxidoreductases is in the range of about 0.5% to about 25%, e.g., about 0.5% to about 20%, about 0.5% to about 15%, about 0.5% to about 10%, about 0.5% to about 7.5%, about 0.5% to about 5%, and about 0.5% to about 4% of total protein. The protein ratio of AA9 polypeptide to catalase is in the range of about 1:1 to about 30:1, e.g., about 1.6:1 to about 10:1 or about 4:1. The protein ratio of AA9 polypeptide to laccase is in the range of about 6:1 to about 300:1, e.g., about 10:1 to about 100:1 or about 20:1. The protein ratio of AA9 polypeptide to peroxidase is in the range of about 1:1 to about 30:1, e.g., about 1.6:1 to about 10:1 or about 4:1.

In another aspect, the combination of the AA9 polypeptide and the one or more oxidoreductases further comprises one or more non-ionic surfactants, cationic surfactants, or non-ionic surfactants and cationic surfactants.

Any nonionic surfactant may be used. The nonionic surfactant may be an alkyl or an aryl surfactant. Examples of nonionic surfactants include glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, and polyoxyethylenated polyoxyproylene glycols, such as EO/PO block copolymers (EO is ethylene oxide, PO is propylene oxide), EO polymers and copolymers, polyamines, and polyvinylpynolidones.

In an embodiment the nonionic surfactant is a linear primary, secondary, or branched alcohol ethoxylate having the formula: RO(CH₂CH₂O)ₙH, wherein R is the hydrocarbon chain length and n is the average number of moles of ethylene oxide, such as where R is linear primary or branched secondary hydrocarbon chain length in the range from C9 to C16 and n ranges from 6 to 13, such as alcohol ethoxylate where R is linear C9-C11 hydrocarbon chain length, and n is 6.

In a preferred embodiment, the nonionic surfactant is nonylphenol ethoxylate. In another preferred embodiment, the nonionic surfactant is C₁₄H₂₂O(C₂H₄O)ₙ. In another preferred embodiment, the nonionic surfactant is C₁₃-alcohol polyethylene glycol ethers (10 EO). In another preferred embodiment, the nonionic surfactant is EO, PO copolymer. In another preferred embodiment, the nonionic surfactant is alkylpolyglycolether. In another preferred embodiment, the nonionic surfactant is RO(EO)₅H. In another preferred embodiment, the nonionic surfactant is HOCH₂(EO)ₙCH₂OH. In another preferred embodiment, the nonionic surfactant is HOCH₂(EO)ₙCH₂OH.

Any cationic surfactant may be used. In an embodiment the cationic surfactant is a primary, secondary, or tertiary amine, such as octenidine dihydrochloride; alkyltrimethylammonium salts, such as cetyl trimethylammonium bromide (CTAB) a.k.a. hexadecyl trimethyl ammonium bromide, cetyl trimethylammonium chloride (CTAC), cetylpyridinium chloride (CPC), benzalkonium chloride (BAC), benzethonium chloride (BZT), 5-bromo-5-nitro-1,3-dioxane, dimethyldioctadecylammonium chloride, dioctadecyldimethylammonium bromide (DODAB) and hexadecyltrimethylammonium bromide.

In a preferred embodiment, the cationic surfactant is C₂₁H₃₈NCl. In another preferred embodiment, the cationic surfactant is CH₃(CH₂)₁₅N(CH₃)₃Br.

In one aspect, the amount of a surfactant is in the range of about 0.01% to about 10% w/w on a dry cellulosic material basis, e.g., about 0.1% to about 7.5%, about 1% to about 5%, about 1% to about 3%, or about 1% to about 2% w/w on a dry cellulosic material basis.

The enzyme compositions may further comprise multiple enzymatic activities, such as one or more (e.g., several) enzymes selected from the group consisting of a cellulase, a hemicellulase, a cellulose inducible protein (CIP), an esterase, an expansin, a ligninolytic enzyme, a pectinase, a protease, and a swollenin. The compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, cellobiohydrolase, cellulase, chitinase, cutinase, cyclodextrin glycosyltransferase, deoxyribonuclease, endoglucanase, esterase, glucoamylase, invertase, lipase, mannosidase, mutanase, oxidase, pectinolytic enzyme, phytase, polyphenoloxidase, proteolytic enzyme, ribonuclease, transglutaminase, or xylanase.

The enzyme composition can also be a fermentation broth formulation or a cell composition. The fermentation broth product further comprises additional ingredients used in the fermentation process, such as, for example, cells, cell debris, biomass, fermentation media and/or fermentation products. In some embodiments, the composition is a cell-killed whole broth containing organic acid(s), killed cells and/or cell debris, and culture medium.

The term “fermentation broth” refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The fermentation broth formulations or cell compositions may further comprise multiple enzymatic activities, such as one or more (e.g., several) enzymes selected from the group consisting of a cellulase, a hemicellulase, a cellulose inducible protein (CIP), an esterase, an expansin, a ligninolytic enzyme, a pectinase, a protease, and a swollenin. The fermentation broth formulations or cell compositions may also comprise one or more (e.g., several) enzymes selected from the group consisting of a hydrolase, an isomerase, a ligase, a lyase, an oxidoreductase, or a transferase, e.g., an alpha-galactosidase, alpha-glucosidase, aminopeptidase, amylase, beta-galactosidase, beta-glucosidase, beta-xylosidase, carbohydrase, carboxypeptidase, cellobiohydrolase, cellulase, chitinase, cutinase, cyclodextrin glycosyltransferase, deoxyribonuclease, endoglucanase, esterase, glucoamylase, invertase, lipase, mannosidase, mutanase, oxidase, pectinolytic enzyme, phytase, polyphenoloxidase, proteolytic enzyme, ribonuclease, transglutaminase, or xylanase.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis (e.g., expression of cellulase and/or glucosidase enzyme(s)). In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The processes of the present invention can be used to saccharify the cellulosic material to fermentable sugars and to convert the fermentable sugars to many useful fermentation products, e.g., fuel (ethanol, n-butanol, isobutanol, biodiesel, jet fuel) and/or platform chemicals (e.g., acids, alcohols, ketones, gases, oils, and the like). The production of a desired fermentation product from the cellulosic material typically involves pretreatment, enzymatic hydrolysis (saccharification), and fermentation.

The processing of the cellulosic material according to the present invention can be accomplished using methods conventional in the art. Moreover, the processes of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze the cellulosic material to fermentable sugars, e.g., glucose, cellobiose, and pentose monomers, and then ferment the fermentable sugars to ethanol. In SSF, the enzymatic hydrolysis of the cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd et al., 2002, Microbiol. Mol. Biol. Reviews 66: 506-577). It is understood herein that any method known in the art comprising pretreatment, enzymatic hydrolysis (saccharification), fermentation, or a combination thereof, can be used in the practicing the processes of the present invention.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

Pretreatment.

In practicing the processes of the present invention, any pretreatment process known in the art can be used to disrupt plant cell wall components of the cellulosic material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40).

The cellulosic material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, the cellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one aspect, the chemical pretreatment is preferably carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt % acid, e.g., 0.05 to 5 wt % acid or 0.1 to 2 wt % acid. The acid is contacted with the cellulosic material and held at a temperature in the range of preferably 140-200° C., e.g., 165-190° C., for periods ranging from 1 to 60 minutes.

In another aspect, pretreatment takes place in an aqueous slurry. In preferred aspects, the cellulosic material is present during pretreatment in amounts preferably between 10-80 wt. %, e.g., 20-70 wt. % or 30-60 wt. %, such as around 40 wt. %. The pretreated cellulosic material can be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in a preferred aspect, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

Saccharification.

In the hydrolysis step, also known as saccharification, the cellulosic material, e.g., pretreated, is hydrolyzed to break down cellulose and/or hemicellulose to fermentable sugars, such as glucose, cellobiose, xylose, xylulose, arabinose, mannose, galactose, and/or soluble oligosaccharides. The hydrolysis is performed enzymatically by one or more enzyme compositions in one or more stages. The hydrolysis can be carried out as a batch process or series of batch processes. The hydrolysis can be carried out as a fed batch or continuous process, or series of fed batch or continuous processes, where the cellulosic or hemicellulosic material is fed gradually to, for example, a hydrolysis solution containing an enzyme composition. In an embodiment the saccharification is a continuous saccharification in which a cellulosic material and a cellulolytic enzyme composition are added at different intervals throughout the saccharification and the hydrolysate is removed at different intervals throughout the saccharification. The removal of the hydrolysate may occur prior to, simultaneously with, or after the addition of the cellulosic material and the cellulolytic enzyme composition.

Enzymatic hydrolysis is preferably carried out in a suitable aqueous environment under conditions that can be readily determined by one skilled in the art. In one aspect, hydrolysis is performed under conditions suitable for the activity of the enzymes(s), i.e., optimal for the enzyme(s).

The saccharification is generally performed in stirred-tank reactors or fermentors under controlled pH, temperature, and mixing conditions. Suitable process time, temperature and pH conditions can readily be determined by one skilled in the art. For example, the total saccharification time can last up to 200 hours, but is typically performed for preferably about 4 to about 120 hours, e.g., about 12 to about 96 hours or about 24 to about 72 hours. The temperature is in the range of preferably about 25° C. to about 80° C., e.g., about 30° C. to about 70° C., about 40° C. to about 60° C., or about 50° C. to about 55° C. The pH is in the range of preferably about 3 to about 9, e.g., about 3.5 to about 8, about 4 to about 7, about 4.2 to about 6, or about 4.3 to about 5.5.

The dry solids content is in the range of preferably about 5 to about 50 wt. %, e.g., about 10 to about 40 wt. % or about 20 to about 30 wt. %.

In one aspect, the degradation or saccharification of the cellulosic material is performed in the presence of dissolved oxygen at a concentration in the range of 0.5 to 10% of the saturation level.

In an embodiment of the invention the dissolved oxygen concentration during degradation or saccharification of the cellulosic material is in the range of 0.5-10% of the saturation level, such as 0.5-7%, such as 0.5-5%, such as 0.5-4%, such as 0.5-3%, such as 0.5-2%, such as 1-5%, such as 1-4%, such as 1-3%, such as 1-2%. In another embodiment, the dissolved oxygen concentration during degradation or saccharification of the cellulosic material is in the range of 0.025 ppm to 0.55 ppm, such as, e.g., 0.05 to 0.165 ppm. In a preferred embodiment, the dissolved oxygen concentration is maintained in the range of 0.5-10% of the saturation level, such as 0.5-7%, such as 0.5-5%, such as 0.5-4%, such as 0.5-3%, such as 0.5-2%, such as 1-5%, such as 1-4%, such as 1-3%, such as 1-2% during at least 25%, such as at least 50% or at least 75% of the degradation or saccharification period.

Oxygen is added to the vessel in order to achieve the desired concentration of dissolved oxygen during saccharification. Maintaining the dissolved oxygen level within a desired range can be accomplished by aeration of the vessel, tank or the like by adding compressed air through a diffuser or sparger, or by other known methods of aeration. The aeration rate can be controlled on the basis of feedback from a dissolved oxygen sensor placed in the vessel/tank, or the system can run at a constant rate without feedback control. In the case of a hydrolysis train consisting of a plurality of vessels/tanks connected in series, aeration can be implemented in one or more or all of the vessels/tanks. Oxygen aeration systems are well known in the art. According to the invention any suitable aeration system may be used. Commercial aeration systems are designed by, e.g., Chemineer, Derby, England, and build by, e.g., Paul Mueller Company, MO, USA.

The enzyme compositions can comprise any protein useful in degrading the cellulosic material.

In one aspect, the enzyme composition comprises or further comprises one or more (e.g., several) proteins selected from the group consisting of a cellulase, an AA9 polypeptide, a hemicellulase, an esterase, an expansin, a ligninolytic enzyme, an oxidoreductase, a pectinase, a protease, and a swollenin. In another aspect, the cellulase is preferably one or more (e.g., several) enzymes selected from the group consisting of an endoglucanase, a cellobiohydrolase, and a beta-glucosidase. In another aspect, the cellulase is preferably one or more (e.g., several) enzymes selected from the group consisting of an endoglucanase, a cellobiohydrolase, a beta-glucosidase, a xylanase, and a beta-xylosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another aspect, the oxidoreductase is preferably one or more (e.g., several) enzymes selected from the group consisting of a catalase, a laccase, and a peroxidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises an endoglucanase and a cellobiohydrolase. In another aspect, the enzyme composition comprises an endoglucanase I, an endoglucanase II, or a combination of an endoglucanase I and an endoglucanase II, and a cellobiohydrolase I, a cellobiohydrolase II, or a combination of a cellobiohydrolase I and a cellobiohydrolase II. In another aspect, the enzyme composition comprises an endoglucanase and a beta-glucosidase. In another aspect, the enzyme composition comprises a beta-glucosidase and a cellobiohydrolase. In another aspect, the enzyme composition comprises a beta-glucosidase and a cellobiohydrolase I, a cellobiohydrolase II, or a combination of a cellobiohydrolase I and a cellobiohydrolase II. In another aspect, the enzyme composition comprises an endoglucanase, a beta-glucosidase, and a cellobiohydrolase. In another aspect, the enzyme composition comprises an endoglucanase I, an endoglucanase II, or a combination of an endoglucanase I and an endoglucanase II, a beta-glucosidase, and a cellobiohydrolase I, a cellobiohydrolase II, or a combination of a cellobiohydrolase I and a cellobiohydrolase II.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In an embodiment, the xylanase is a Family 10 xylanase. In another embodiment, the xylanase is a Family 11 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a ligninolytic enzyme. In an embodiment, the ligninolytic enzyme is a manganese peroxidase. In another embodiment, the ligninolytic enzyme is a lignin peroxidase. In another embodiment, the ligninolytic enzyme is a H₂O₂-producing enzyme. In another aspect, the enzyme composition comprises a pectinase. In another aspect, the enzyme composition comprises a protease. In another aspect, the enzyme composition comprises a swollenin.

In the processes of the present invention, the enzyme(s) can be added prior to or during saccharification, saccharification and fermentation, or fermentation.

One or more (e.g., several) components of the enzyme composition may be native proteins, recombinant proteins, or a combination of native proteins and recombinant proteins. For example, one or more (e.g., several) components may be native proteins of a cell, which is used as a host cell to express recombinantly one or more (e.g., several) other components of the enzyme composition. It is understood herein that the recombinant proteins may be heterologous (e.g., foreign) and/or native to the host cell. One or more (e.g., several) components of the enzyme composition may be produced as monocomponents, which are then combined to form the enzyme composition. The enzyme composition may be a combination of multicomponent and monocomponent protein preparations.

The enzymes used in the processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes depend on several factors including, but not limited to, the mixture of cellulolytic enzymes and/or hemicellulolytic enzymes, the cellulosic material, the concentration of cellulosic material, the pretreatment(s) of the cellulosic material, temperature, time, pH, and inclusion of a fermenting organism (e.g., for Simultaneous Saccharification and Fermentation).

In one aspect, an effective amount of cellulolytic or hemicellulolytic enzyme to the cellulosic material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg of protein per g of the cellulosic material.

In another aspect, an effective amount of an AA9 polypeptide to the cellulosic material is about 0.01 to about 50.0 mg, e.g., about 0.01 to about 40 mg, about 0.01 to about 30 mg, about 0.01 to about 20 mg, about 0.01 to about 10 mg, about 0.01 to about 5 mg, about 0.025 to about 1.5 mg, about 0.05 to about 1.25 mg, about 0.075 to about 1.25 mg, about 0.1 to about 1.25 mg, about 0.15 to about 1.25 mg, or about 0.25 to about 1.0 mg of protein per g of the cellulosic material.

In another aspect, an effective amount of a laccase to the cellulosic material is about 0.001 to about 5.0 mg, e.g., about 0.001 to about 4 mg, about 0.001 to about 3 mg, about 0.001 to about 2 mg, about 0.001 to about 1 mg, about 0.001 to about 0.5 mg, about 0.002 to about 0.25 mg, about 0.005 to about 0.125 mg, about 0.075 to about 0.06 mg of protein per g of the cellulosic material.

In another aspect, an effective amount of a catalase to the cellulosic material is about 0.001 to about 10.0 mg, e.g., about 0.001 to about 5 mg, about 0.001 to about 4 mg, about 0.001 to about 3 mg, about 0.001 to about 2 mg, about 0.001 to about 1 mg, about 0.005 to about 5 mg, about 0.025 to about 2.5 mg, about 0.025 to about 1.25 mg, about 0.05 to about 0.5 mg, or about 0.05 to about 0.25 mg protein per g of the cellulosic material.

In another aspect, an effective amount of a peroxidase to the cellulosic material is about 0.001 to about 10.0 mg, e.g., about 0.001 to about 5 mg, about 0.001 to about 4 mg, about 0.001 to about 3 mg, about 0.001 to about 2 mg, about 0.001 to about 1 mg, about 0.005 to about 5 mg, about 0.025 to about 2.5 mg, about 0.025 to about 1.25 mg, about 0.05 to about 0.5 mg, or about 0.05 to about 0.25 mg protein per g of the cellulosic material.

The polypeptides having cellulolytic enzyme activity or hemicellulolytic enzyme activity as well as other proteins/polypeptides useful in the degradation of the cellulosic or hemicellulosic material, e.g., AA9 polypeptides can be derived or obtained from any suitable origin, including, archaeal, bacterial, fungal, yeast, plant, or animal origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained by, e.g., site-directed mutagenesis or shuffling. Each polypeptide may be a bacterial polypeptide. For example, each polypeptide may be a Gram-positive bacterial polypeptide having enzyme activity, or a Gram-negative bacterial polypeptide having enzyme activity.

Each polypeptide may also be a fungal polypeptide, e.g., a yeast polypeptide or a filamentous fungal polypeptide.

Chemically modified or protein engineered mutants of polypeptides may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host can be a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec (Novozymes NS), CELLIC® CTec2 (Novozymes NS), CELLIC® CTec3 (Novozymes NS), CELLUCLAST™ (Novozymes NS), NOVOZYM™ 188 (Novozymes NS), SPEZYME™ CP (Genencor Int.), ACCELLERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.). The cellulolytic enzyme preparation is added in an amount effective from about 0.001 to about 5.0 wt. % of solids, e.g., about 0.025 to about 4.0 wt. % of solids or about 0.005 to about 2.0 wt. % of solids.

Examples of bacterial endoglucanases that can be used in the processes of the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (GenBank:XM_324477), Humicola insolens endoglucanase V, Myceliophthora thermophila CBS 117.65 endoglucanase, Thermoascus aurantiacus endoglucanase I (GenBank:AF487830), Trichoderma reesei strain No. VTT-D-80133 endoglucanase (GenBank:M15665), and Penicillium pinophilum endoglucanase (WO 2012/062220).

Examples of cellobiohydrolases useful in the present invention include, but are not limited to, Aspergillus aculeatus cellobiohydrolase II (WO 2011/059740), Aspergillus fumigatus cellobiohydrolase I (WO 2013/028928), Aspergillus fumigatus cellobiohydrolase II (WO 2013/028928), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (GenBank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

Other useful endoglucanases, cellobiohydrolases, and beta-glucosidases are disclosed in numerous Glycosyl Hydrolase families using the classification according to Henrissat, 1991, Biochem. J. 280: 309-316, and Henrissat and Bairoch, 1996, Biochem. J. 316: 695-696.

In the processes of the present invention, any AA9 polypeptide can be used as a component of the enzyme composition as described in the AA9 Polypeptides section herein.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes NS), CELLIC® HTec (Novozymes NS), CELLIC® HTec2 (Novozymes NS), CELLIC® HTec3 (Novozymes NS), VISCOZYME® (Novozymes NS), ULTRAFLO® (Novozymes NS), PULPZYME® HC (Novozymes NS), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Thermomyces lanuginosus (GeneSeqP:BAA22485), Talaromyces thermophilus (GeneSeqP:BAA22834), Thielavia terrestris NRRL 8126 (WO 2009/079210), and Trichophaea saccata (WO 2011/057083).

Examples of beta-xylosidases useful in the processes of the present invention include, but are not limited to, beta-xylosidases from Neurospora crassa (SwissProt:Q7SOW4), Trichoderma reesei (UniProtKB/TrEMBL:Q92458), Talaromyces emersonii (SwissProt:Q8X212), and Talaromyces thermophilus (GeneSeqP:BAA22816).

Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1 D9T4), Neurospora crassa (UniProt:Q9HGR3), Penicillium aurantiogriseum (WO 2009/127729), and Thielavia terrestris (WO 2010/053838 and WO 2010/065448).

Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (SwissProt:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Examples of oxidoreductases useful in the processes of the present invention are described in the Oxidoreductases Section herein.

The polypeptides having enzyme activity used in the processes of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation.

The fermentable sugars obtained from the hydrolyzed cellulosic material can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous.

Any suitable hydrolyzed cellulosic material can be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Xylose fermenting yeast include strains of Candida, preferably C. sheatae or C. sonorensis; and strains of Pichia, e.g., P. stipitis, such as P. stipitis CBS 5773. Pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, G. P., 1996, Cellulose bioconversion technology, in Handbook on Bioethanol: Production and Utilization, Wyman, C. E., ed., Taylor & Francis, Washington, D.C., 179-212).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIO-FERM® AFT and XR (Lallemand Specialities, Inc., USA), ETHANOL RED® yeast (Lesaffre et Co, pagnie, France), FALI® (AB Mauri Food Inc., USA), FERMIOL® (Rymco International AG, Denmark), GERT STRAND™ (Gert Strand AB, Sweden), and SUPERSTART™ and THERMOSACC® fresh yeast (Lallemand Specialities, Inc., USA).

In an aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (co-fermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

It is well known in the art that the organisms described above can also be used to produce other substances, as described herein.

The fermenting microorganism is typically added to the degraded cellulosic material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded cellulosic material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation Products:

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In one aspect, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another aspect, the fermentation product is an alkane. The alkane may be an unbranched or a branched alkane. The alkane can be, but is not limited to, pentane, hexane, heptane, octane, nonane, decane, undecane, or dodecane.

In another aspect, the fermentation product is a cycloalkane. The cycloalkane can be, but is not limited to, cyclopentane, cyclohexane, cycloheptane, or cyclooctane.

In another aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another aspect, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another aspect, the fermentation product is isoprene.

In another aspect, the fermentation product is a ketone. The term “ketone” encompasses a substance that contains one or more ketone moieties. The ketone can be, but is not limited to, acetone.

In another aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another aspect, the fermentation product is polyketide.

Recovery.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### AA9 Polypeptides Having Cellulolytic Enhancing Activity and Polynucleotides Thereof

In the processes of the present invention, any AA9 polypeptide having cellulolytic enhancing activity may be used. See, for example, SEQ ID NOs: 1-86.

Examples of AA9 polypeptides useful in the processes of the present invention include, but are not limited to, AA9 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290 and WO 2012/149344), Myceliophthora thermophila (WO 2009/085935, WO 2009/085859, WO 2009/085864, WO 2009/085868, and WO 2009/033071), Aspergillus fumigatus (WO 2010/138754), Penicillium pinophilum (WO 2011/005867), Thermoascus sp. (WO 2011/039319), Penicillium sp. (emersonii (WO 2011/041397 and WO 2012/000892), Thermoascus crustaceous (WO 2011/041504), Aspergillus aculeatus (WO 2012/125925), Thermomyces lanuginosus (WO 2012/113340, WO 2012/129699, WO 2012/130964, and WO 2012/129699), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206), and Talaromyces thermophilus (WO 2012/129697 and WO 2012/130950).

Non-limiting examples of AA9 polypeptides having cellulolytic enhancing activity useful in the present invention are AA9 polypeptides from Thielavia terrestris (GeneSeqP:AEB90517, AEB90519, AEB90521, AEB90523, AEB90525, or AUM21652), Thermoascus aurantiacus (GeneSeqP:AZJ19467), Trichoderma reesei (GeneSeqP:AFY26868 or BAF28697), Myceliophthora thermophila (GeneSeqP:AXD75715, AXD75717, AXD58945, AXD80944, AXF00393), Thermoascus aurantiacus (GeneSeqP:AYD12322), Aspergillus fumigatus (GeneSeqP:AYM96878); Penicillium pinophilum (GeneSeqP:AYN30445), Thermoascus sp. (GeneSeqP:AZG48808), Penicillium sp. (emersonii) (GeneSeqP:AZG65226), Thielavia terrestris (GeneSeqP:AZG26658, AZG26660, AZG26662, AZG26664, AZG26666, AZG26668, AZG26670, AZG26672, AZG26674, AZG26676, or AZG26678), Thermoascus crustaceus(GeneSeqP:AZG67666, AZG67668, or AZG67670), Aspergillus aculeatus (GeneSeqP:AZT94039, AZT94041, AZT94043, AZT94045, AZT94047, AZT94049, or AZT94051), Thermomyces lanuginosus (GeneSeqP:AZZ14902, AZZ14904, or AZZ14906), Aurantiporus alborubescens (GeneSeqP: AZZ98498 or AZZ98500), Trichophaea saccata (GeneSeqP:AZZ98502 or AZZ98504), Penicillium thomii (GeneSeqP:AZZ98506), Talaromyces stipitatus (GeneSeqP:BAD71945), Humicola insolens (GeneSeqP:BAE45292, BAE45294, BAE45296, BAE45298, BAE45300, BAE45302, BAE45304, BAE45306, BAE45308, BAE45310, BAE45312, BAE45314, BAE45316, BAE45318, BAE45320, BAE45322, BAE45324, BAE45326, BAE45328, BAE45330, BAE45332, BAE45334, BAE45336, BAE45338, BAE45340, BAE45342, or BAE45344), Malbranchea cinnamomea (GeneSeqP:AZY42250), Talaromyces leycettanus (GeneSeqP:AZY42258), and Chaetomium thermophilum (GeneSeqP:AZY42252). The accession numbers are incorporated herein in their entirety.

In one aspect, the AA9 polypeptide has a sequence identity to the mature polypeptide of any of the AA9 polypeptides disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have cellulolytic enhancing activity.

In another aspect, the amino acid sequence of the AA9 polypeptide differs by up to 10 amino acids, e.g., 1, 2, 3, 4, 5, 6, 7, 8, 9, or 10 from the mature polypeptide of any of the AA9 polypeptides disclosed herein.

In another aspect, the AA9 polypeptide comprises or consists of the amino acid sequence of any of the AA9 polypeptides disclosed herein.

In another aspect, the AA9 polypeptide comprises or consists of the mature polypeptide of any of the AA9 polypeptides disclosed herein.

In another embodiment, the AA9 polypeptide is an allelic variant of an AA9 polypeptide disclosed herein.

In another aspect, the AA9 polypeptide is a fragment containing at least 85% of the amino acid residues, e.g., at least 90% of the amino acid residues or at least 95% of the amino acid residues of the mature polypeptide of an AA9 polypeptide disclosed herein.

In another aspect, the AA9 polypeptide is encoded by a polynucleotide that hybridizes under very low stringency conditions, low stringency conditions, medium stringency conditions, medium-high stringency conditions, high stringency conditions, or very high stringency conditions with the mature polypeptide coding sequence or the full-length complement thereof of any of the AA9 polypeptides disclosed herein (Sambrook et al., 1989, Molecular Cloning, A Laboratory Manual, 2d edition, Cold Spring Harbor, N.Y.).

The polynucleotide encoding an AA9 polypeptide, or a subsequence thereof, as well as the polypeptide of an AA9 polypeptide, or a fragment thereof, may be used to design nucleic acid probes to identify and clone DNA encoding an AA9 polypeptide from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes an AA9 polypeptide. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with such a nucleic acid probe, the carrier material is used in a Southern blot.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of an AA9 polypeptide.

In another aspect, the nucleic acid probe is a polynucleotide that encodes a full-length AA9 polypeptide; the mature polypeptide thereof; or a fragment thereof.

In another aspect, the AA9 polypeptide is encoded by a polynucleotide having a sequence identity to the mature polypeptide coding sequence of any of the AA9 polypeptides disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The AA9 polypeptide may be a hybrid polypeptide in which a region of one polypeptide is fused at the N-terminus or the C-terminus of a region of another polypeptide.

The AA9 polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The AA9 polypeptide may be obtained from microorganisms of any genus. For purposes of the present invention, the term “obtained from” as used herein in connection with a given source shall mean that the AA9 polypeptide encoded by a polynucleotide is produced by the source or by a strain in which the polynucleotide from the source has been inserted. In one embodiment, the AA9 polypeptide is secreted extracellularly.

The AA9 polypeptide may be a bacterial AA9 polypeptide. For example, the AA9 polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces AA9 polypeptide, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasman AA9 polypeptide.

The AA9 polypeptide may be a fungal AA9 polypeptide. For example, the AA9 polypeptide may be a yeast AA9 polypeptide such as a Candida, Kluyveromyces, Pichia, Saccharomyces, Schizosaccharomyces, or Yarrowian AA9 polypeptide; or a filamentous fungal AA9 polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylarian AA9 polypeptide.

The AA9 polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding an AA9 polypeptide may then be obtained by similarly screening a genomic DNA or cDNA library of another microorganism or mixed DNA sample. Once a polynucleotide encoding an AA9 polypeptide has been detected with the probe(s), the polynucleotide can be isolated or cloned by utilizing techniques that are known to those of ordinary skill in the art (see, e.g., Sambrook et al., 1989, supra).

In one aspect, the AA9 polypeptide is used in the presence of a soluble activating divalent metal cation according to WO 2008/151043 or WO 2012/122518, e.g., manganese or copper.

In another aspect, the AA9 polypeptide is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

In one aspect, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁶ to about 1, about 10⁻⁶ to about 10⁻¹, about 10⁻⁴ to about 10⁻¹, about 10⁻³ to about 10⁻¹, or about 10⁻³ to about 10⁻². In another aspect, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described in WO 2012/021401, and the soluble contents thereof. A liquor for cellulolytic enhancement of an AA9 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one aspect, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁶ to about 1 g, about 10⁻⁶ to about 10⁻¹ g, about 10⁻⁴ to about 10⁻¹ g, about 10⁻³ to about 10⁻¹ g, or about 10⁻³ to about 10⁻² g per g of cellulose.

### Oxidoreductases

In the processes of the present invention, the one or more oxidoreductases are independently selected from the group consisting of catalases, laccases, and peroxidases. Any catalase, laccase, and/or peroxidase may be used. See, for example, SEQ ID NOs: 87-94.

Catalases

The catalase may be any catalase useful in the processes of the present invention. The catalase may include, but is not limited to, an E.C. 1.11.1.6 or E.C. 1.11.1.21 catalase.

Examples of useful catalases include, but are not limited to, catalases from Alcaligenes aquamarinus (WO 98/00526), Aspergillus lentilus, Aspergillus fumigatus, Aspergillus niger (U.S. Pat. No. 5,360,901), Aspergillus oryzae (JP 2002223772A; U.S. Pat. No. 6,022,721), Bacillus thermoglucosidasius (JP 1 1243961A), Humicola insolens (WO 2009/104622, WO 2012/130120), Malbranchea cinnamomea, Microscilla furvescens (WO 98/00526), Neurospora crassa, Penicillium emersonii (WO 2012/130120), Penicillium pinophilum, Rhizomucor pusillus, Saccharomyces pastorianus (WO 2007/105350), Scytalidium thermophilum, Talaromyces stipitatus (WO 2012/130120), Thermoascus aurantiacus (WO 2012/130120), Thermus brockianus (WO 2005/044994), and Thielavia terrestris (WO 2010/074972).

Non-limiting examples of catalases useful in the present invention are catalases from Bacillus pseudofirmus (UNIPROT: P30266), Bacillus subtilis (UNIPROT:P42234), Humicola grisea (GeneSeqP: AXQ55105), Neosartorya fischeri (UNIPROT:A1DJU9), Penicillium emersonii (GeneSeqP:BAC10987), Penicillium pinophilum (GeneSeqP:BAC10995), Scytalidium thermophilum (GeneSeqP:AAW06109 or ADT89624), Talaromyces stipitatus (GeneSeqP:BAC10983 or BAC11039; UNIPROT:B8MT74), and Thermoascus aurantiacus (GeneSeqP:BAC11005). The accession numbers are incorporated herein in their entirety.

In one aspect, the catalase has a sequence identity to the mature polypeptide of any of the catalases disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have catalase activity.

In another aspect, the amino acid sequence of the catalase differs by up to 10 amino acids, e.g., 1, 2, 3, 4, 5, 6, 7, 8, 9, or 10 from the mature polypeptide of any of the catalases disclosed herein.

In another aspect, the catalase comprises or consists of the amino acid sequence of any of the catalases disclosed herein.

In another aspect, the catalase comprises or consists of the mature polypeptide of any of the catalases disclosed herein.

In another embodiment, the catalase is an allelic variant of a catalase disclosed herein.

In another aspect, the catalase is a fragment containing at least 85% of the amino acid residues, e.g., at least 90% of the amino acid residues or at least 95% of the amino acid residues of the mature polypeptide of a catalase disclosed herein.

In another aspect, the catalase is encoded by a polynucleotide that hybridizes under very low stringency conditions, low stringency conditions, medium stringency conditions, medium-high stringency conditions, high stringency conditions, or very high stringency conditions with the mature polypeptide coding sequence or the full-length complement thereof of any of the catalases disclosed herein (Sambrook et al., 1989, supra).

The polynucleotide encoding a catalase, or a subsequence thereof, as well as the polypeptide of a catalase, or a fragment thereof, may be used to design nucleic acid probes to identify and clone DNA encoding a catalase from strains of different genera or species according to methods well known in the art. In particular, such probes can be used for hybridization with the genomic DNA or cDNA of a cell of interest, as described supra.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of a catalase.

In another aspect, the nucleic acid probe is a polynucleotide that encodes a full-length catalase; the mature polypeptide thereof; or a fragment thereof.

In another aspect, the catalase is encoded by a polynucleotide having a sequence identity to the mature polypeptide coding sequence of any of the catalases disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The catalase may be a hybrid polypeptide in which a region of one polypeptide is fused at the N-terminus or the C-terminus of a region of another polypeptide or a fusion polypeptide or cleavable fusion polypeptide in which another polypeptide is fused at the N-terminus or the C-terminus of the catalase, as described herein.

Laccases

The laccase may be any laccase useful in the processes of the present invention. The laccase may include, but is not limited to, an E.C. 1.10.3.2 laccase.

Examples of useful laccases include, but are not limited to, laccases from Chaetomium thermophilum, Coprinus cinereus, Coriolus versicolor, Melanocarpus albomyces, Myceliophthora thermophila, Polyporus pinsitus, Pycnoporus cinnabarinus, Rhizoctonia solani, Scytalidium thermophilum, and Streptomyces coelicolor.

Non-limiting examples of laccases useful in the present invention are laccases from Chaetomium thermophilum (GeneSeqP:AEH03373), Coprinus cinereus (GeneSeqP:AAW17973 or AAW17975), Coriolus versicolor (GeneSeqP:ABR57646), Melanocarpus albomyces (GeneSeqP:AAU76464), Myceliophthora thermophila (GeneSeqP:AAW19855), Polyporus pinsitus (GeneSeqP:AAR90721), Rhizoctonia solani GeneSeqP:AAW60879 or AAW60925), and Scytalidium thermophilum (GeneSeqP:AAW18069 or AAW51783). The accession numbers are incorporated herein in their entirety.

In one aspect, the laccase has a sequence identity to the mature polypeptide of any of the laccases disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have laccase activity.

In another aspect, the amino acid sequence of the laccase differs by up to 10 amino acids, e.g., 1, 2, 3, 4, 5, 6, 7, 8, 9, or 10 from the mature polypeptide of any of the laccases disclosed herein.

In another aspect, the laccase comprises or consists of the amino acid sequence of any of the laccases disclosed herein.

In another aspect, the laccase comprises or consists of the mature polypeptide of any of the laccases disclosed herein.

In another embodiment, the laccase is an allelic variant of a laccase disclosed herein.

In another aspect, the laccase is a fragment containing at least 85% of the amino acid residues, e.g., at least 90% of the amino acid residues or at least 95% of the amino acid residues of the mature polypeptide of a laccase disclosed herein.

In another aspect, the laccase is encoded by a polynucleotide that hybridizes under very low stringency conditions, low stringency conditions, medium stringency conditions, medium-high stringency conditions, high stringency conditions, or very high stringency conditions with the mature polypeptide coding sequence or the full-length complement thereof of any of the laccases disclosed herein (Sambrook et al., 1989, supra).

The polynucleotide encoding a laccase, or a subsequence thereof, as well as the polypeptide of a laccase, or a fragment thereof, may be used to design nucleic acid probes to identify and clone DNA encoding a laccase from strains of different genera or species according to methods well known in the art. In particular, such probes can be used for hybridization with the genomic DNA or cDNA of a cell of interest, as described supra.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of a laccase.

In another aspect, the nucleic acid probe is a polynucleotide that encodes a full-length laccase; the mature polypeptide thereof; or a fragment thereof.

In another aspect, the laccase is encoded by a polynucleotide having a sequence identity to the mature polypeptide coding sequence of any of the laccases disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The laccase may be a hybrid polypeptide in which a region of one polypeptide is fused at the N-terminus or the C-terminus of a region of another polypeptide or a fusion polypeptide or cleavable fusion polypeptide in which another polypeptide is fused at the N-terminus or the C-terminus of the laccase, as described herein.

Peroxidases

The peroxidase may be any peroxidase useful in the processes of the present invention. The peroxidase may include, but not limited to, E.C. 1.11.1.1 NADH peroxidase, E.C. 1.11.1.2 NADPH peroxidase, E.C. 1.11.1.3 fatty acid peroxidase, E.C. 1.11.1.5 di-heme cytochrome c peroxidase, E.C. 1.11.1.5 cytochrome c peroxidase, E.C. 1.11.1.7 invertebrate peroxinectin, E.C. 1.11.1.7 eosinophil peroxidase, E.C. 1.11.1.7 lactoperoxidase, E.C. 1.11.1.7 myeloperoxidase, E.C. 1.11.1.8 thyroid peroxidase, E.C. 1.11.1.9 glutathione peroxidase, E.C. 1.11.1.10 chloride peroxidase, E.C. 1.11.1.11 ascorbate peroxidase, E.C. 1.11.1.12 other glutathione peroxidase, E.C. 1.11.1.13 manganese peroxidase, E.C. 1.11.1.14 lignin peroxidase, E.C. 1.11.1.15 cysteine peroxiredoxin, E.C. 1.11.1.16 versatile peroxidase, E.C. 1.11.1.B2 chloride peroxidase, E.C. 1.11.1.B4 haloperoxidase, E.C. 1.11.1.B4 no-heme vanadium haloperoxidase, E.C. 1.11.1.B6 iodide peroxidase, E.C. 1.11.1.B7 bromide peroxidase, and E.C. 1.11.1.B8 iodide peroxidase.

In one embodiment, the peroxidase is a NADH peroxidase. In another embodiment, the peroxidase is a NADPH peroxidase. In another embodiment, the peroxidase is a fatty acid peroxidase. In another embodiment, the peroxidase is a di-heme cytochrome c peroxidase. In another embodiment, the peroxidase is a cytochrome c peroxidase. In another embodiment, the peroxidase is a catalase. In another embodiment, the peroxidase is a manganese catalase. In another embodiment, the peroxidase is an invertebrate peroxinectin. In another embodiment, the peroxidase is an eosinophil peroxidase. In another embodiment, the peroxidase is a lactoperoxidase. In another embodiment, the peroxidase is a myeloperoxidase. In another embodiment, the peroxidase is a thyroid peroxidase. In another embodiment, the peroxidase is a glutathione peroxidase. In another embodiment, the peroxidase is a chloride peroxidase. In another embodiment, the peroxidase is an ascorbate peroxidase. In another embodiment, the peroxidase is a glutathione peroxidase. In another embodiment, the peroxidase is a manganese peroxidase. In another embodiment, the peroxidase is a lignin peroxidase. In another embodiment, the peroxidase is a cysteine peroxiredoxin. In another embodiment, the peroxidase is a versatile peroxidase. In another embodiment, the peroxidase is a chloride peroxidase. In another embodiment, the peroxidase is a haloperoxidase. In another embodiment, the peroxidase is a no-heme vanadium haloperoxidase. In another embodiment, the peroxidase is an iodide peroxidase. In another embodiment, the peroxidase is a bromide peroxidase. In another embodiment, the peroxidase is a iodide peroxidase.

Examples of useful peroxidases include, but are not limited to, Coprinus cinereus peroxidase (Baunsgaard et al., 1993, Amino acid sequence of Coprinus macrorhizus peroxidase and cDNA sequence encoding Coprinus cinereus peroxidase. A new family of fungal peroxidases, Eur. J. Biochem. 213 (1): 605-611 (Accession number P28314); horseradish peroxidase (Fujiyama et al., 1988, Structure of the horseradish peroxidase isozyme C genes, Eur. J. Biochem. 173 (3): 681-687 (Accession number P15232); peroxiredoxin (Singh and Shichi, 1998, A novel glutathione peroxidase in bovine eye. Sequence analysis, mRNA level, and translation, J. Biol. Chem. 273 (40): 26171-26178 (Accession number O77834); lactoperoxidase (Dull et al., 1990, Molecular cloning of cDNAs encoding bovine and human lactoperoxidase, DNA Cell Biol. 9 (7): 499-509 (Accession number P80025); Eosinophil peroxidase (Fornhem et al., 1996, Isolation and characterization of porcine cationic eosinophil granule proteins, Int. Arch. Allergy Immunol. 110 (2): 132-142 (Accession number P80550); versatile peroxidase (Ruiz-Duenas et al., 1999, Molecular characterization of a novel peroxidase isolated from the ligninolytic fungus Pleurotus eryngii, Mol. Microbiol. 31 (1): 223-235 (Accession number O94753); turnip peroxidase (Mazza and Welinder, 1980, Covalent structure of turnip peroxidase 7. Cyanogen bromide fragments, complete structure and comparison to horseradish peroxidase C, Eur. J. Biochem. 108 (2): 481-489 (Accession number P00434); myeloperoxidase (Morishita et al., 1987, Chromosomal gene structure of human myeloperoxidase and regulation of its expression by granulocyte colony-stimulating factor, J. Biol. Chem. 262 (31): 15208-15213 (Accession number P05164); peroxidasin and peroxidasin homologs (Horikoshi et al., 1999, Isolation of differentially expressed cDNAs from p53-dependent apoptotic cells: activation of the human homologue of the Drosophila peroxidasin gene, Biochem. Biophys. Res. Commun. 261 (3): 864-869 (Accession number Q92626); lignin peroxidase (Tien and Tu, 1987, Cloning and sequencing of a cDNA for a ligninase from Phanerochaete chrysosporium, Nature 326 (6112): 520-523 (Accession number P06181); Manganese peroxidase (Orth et al., 1994, Characterization of a cDNA encoding a manganese peroxidase from Phanerochaete chrysosporium: genomic organization of lignin and manganese peroxidase-encoding genes, Gene 148 (1): 161-165 (Accession number P78733); Soy peroxidase, Royal palm peroxidase, alpha-dioxygenase, dual oxidase, peroxidasin, invertebrate peroxinectin, short peroxidockerin, lactoperoxidase, myeloperoxidase, non-mammalian vertebrate peroxidase, catalase, catalase-lipoxygenase fusion, di-heme cytochrome c peroxidase, methylamine utilization protein, DyP-type peroxidase, haloperoxidase, ascorbate peroxidase, catalase peroxidase, hybrid ascorbate-cytochrome c peroxidase, lignin peroxidase, manganese peroxidase, versatile peroxidase, other class II peroxidase, class III peroxidase, alkylhydroperoxidase D, other alkylhydroperoxidases, no-heme, no metal haloperoxidase, no-heme vanadium haloperoxidase, manganese catalase, NADH peroxidase, glutathione peroxidase, cysteine peroxiredoxin, thioredoxin-dependent thiol peroxidase, and AhpE-like peroxiredoxin (Passard et al., 2007, Phytochemistry 68:1605-1611.

Non-limiting examples of peroxidases useful in the present invention are peroxidases from Coprinus cinereus (GeneSeqP:AAR75422), soybean (GeneSeqP:AZY11808), Royal palm tree (GeneSeqP:AZY11826), and Zea mays (GeneSeqP:AZY11858) peroxidase. The accession numbers are incorporated herein in their entirety.

In one aspect, the peroxidase has a sequence identity to the mature polypeptide of any of the peroxidases disclosed herein of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have peroxidase activity.

In another aspect, the amino acid sequence of the peroxidase differs by up to 10 amino acids, e.g., 1, 2, 3, 4, 5, 6, 7, 8, 9, or 10 from the mature polypeptide of any of the peroxidases disclosed herein.

In another aspect, the peroxidase comprises or consists of the amino acid sequence of any of the peroxidases disclosed herein.

In another aspect, the peroxidase comprises or consists of the mature polypeptide of any of the peroxidases disclosed herein.

In another embodiment, the peroxidase is an allelic variant of a peroxidase disclosed herein.

In another aspect, the peroxidase is a fragment containing at least 85% of the amino acid residues, e.g., at least 90% of the amino acid residues or at least 95% of the amino acid residues of the mature polypeptide of a peroxidase disclosed herein.

In another aspect, the peroxidase is encoded by a polynucleotide that hybridizes under very low stringency conditions, low stringency conditions, medium stringency conditions, medium-high stringency conditions, high stringency conditions, or very high stringency conditions with the mature polypeptide coding sequence or the full-length complement thereof of any of the peroxidases disclosed herein (Sambrook et al., 1989, supra).

The polynucleotide encoding a peroxidase, or a subsequence thereof, as well as the polypeptide of a peroxidase, or a fragment thereof, may be used to design nucleic acid probes to identify and clone DNA encoding a peroxidase from strains of different genera or species according to methods well known in the art. In particular, such probes can be used for hybridization with the genomic DNA or cDNA of a cell of interest, as described supra.

For purposes of the present invention, hybridization indicates that the polynucleotide hybridizes to a labeled nucleic acid probe under very low to very high stringency conditions. Molecules to which the nucleic acid probe hybridizes under these conditions can be detected using, for example, X-ray film or any other detection means known in the art.

In one aspect, the nucleic acid probe is the mature polypeptide coding sequence of a peroxidase.

In another aspect, the nucleic acid probe is a polynucleotide that encodes a full-length peroxidase; the mature polypeptide thereof; or a fragment thereof.

In another aspect, the peroxidase is encoded by a polynucleotide having a sequence identity to the mature polypeptide coding sequence of a peroxidase of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

The peroxidase may be a hybrid polypeptide in which a region of one polypeptide is fused at the N-terminus or the C-terminus of a region of another polypeptide or a fusion polypeptide or cleavable fusion polypeptide in which another polypeptide is fused at the N-terminus or the C-terminus of the peroxidase, as described herein.

In each of the embodiments above, the oxidoreductase may be obtained from microorganisms, plants, or animals of any genus. In one aspect, the oxidoreductase obtained from a given source is secreted extracellularly.

The oxidoreductase may be a bacterial oxidoreductase. For example, the oxidoreductase may be a gram positive bacterial oxidoreductase such as a Bacillus, Streptococcus, Streptomyces, Staphylococcus, Enterococcus, Lactobacillus, Lactococcus, Clostridium, Geobacillus, or Oceanobacillus oxidoreductase, or a Gram negative bacterial oxidoreductase such as an E. coli, Pseudomonas, Salmonella, Campylobacter, Helicobacter, Flavobacterium, Fusobacterium, Ilyobacter, Neisseria, or Ureaplasma oxidoreductase.

In one aspect, the oxidoreductase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis oxidoreductase.

In another aspect, the oxidoreductase is a Streptococcus equisimilis, Streptococcus pyogenes, Streptococcus uberis, or Streptococcus equi subsp. Zooepidemicus oxidoreductase.

In another aspect, the oxidoreductase is a Streptomyces achromogenes, Streptomyces avermitilis, Streptomyces coelicolor, Streptomyces griseus, or Streptomyces lividans oxidoreductase.

The oxidoreductase may also be a fungal oxidoreductase, and more preferably a yeast oxidoreductase such as a Candida, Kluyveromyces, Pichia, Saccharomyces, Schizosaccharomyces, or Yarrowia oxidoreductase; or more preferably a filamentous fungal oxidoreductase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria oxidoreductase.

In another aspect, the oxidoreductase is a Saccharomyces carlsbergensis, Saccharomyces cerevisiae, Saccharomyces diastaticus, Saccharomyces douglasfi, Saccharomyces kluyveri, Saccharomyces norbensis, or Saccharomyces oviformis oxidoreductase.

In another aspect, the oxidoreductase is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium emersonii, Penicillium funiculosum, Penicillium purpurogenum, Phanerochaete chrysosporium, Polyporus pinsitus, Thermoascus aurantiacus, Thermoascus crustaceus, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride oxidoreductase.

The oxidoreductase may be a plant oxidoreductase. In another aspect, the oxidoreductase is horseradish oxidoreductase. In another aspect, the oxidoreductase is soybean oxidoreductase.

Techniques used to isolate or clone a polynucleotide encoding a oxidoreductase are known in the art and include isolation from genomic DNA, preparation from cDNA, or a combination thereof. The cloning of the polynucleotides of the present invention from such genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and nucleotide sequence-based amplification (NASBA) may be used.

The present invention is further described by the following examples that should not be construed as limiting the scope of the invention.

### EXAMPLES

### Example 1

### Preparation of Enzymes

Humicola insolens endoglucanase V core was obtained from Novozymes NS (Bagsvaerd, Denmark) as CAREZYME CORE™

Aspergillus fumigatus cellobiohydrolase I (GeneSeqP:AZI04842; SEQ ID NO: 87) can be prepared according to WO 2011/057140.

Aspergillus fumigatus cellobiohydrolase II (GeneSeqP:AZI04854; SEQ ID NO: 88) can be prepared according to WO 2011/057140.

Thermoascus aurantiacus AA9 (GH61A) polypeptide (GeneSeqP:AZJ19467; SEQ ID NO: 7) was prepared according to WO 2005/074656.

Penicillium sp. (emersonii) AA9 (GH61A) polypeptide (GeneSeqP:AZG65226; SEQ ID NO: 18) was recombinantly prepared according to WO 2011/041397 using Trichoderma reesei as host. The filtered broth of the Penicillium sp. (emersonii) GH61A polypeptide was buffer exchanged into 20 mM Tris pH 8.5 using a 400 ml Sephadex® G-25 column (GE Healthcare, United Kingdom) according to the manufacturer's instructions. The protein was applied to a Q SEPHAROSE® Fast Flow column (GE Healthcare, Piscataway, N.J., USA) equilibrated in 20 mM Tris pH 8.5, and bound proteins were eluted using a linear gradient from 0-600 mM sodium chloride. The eluted protein fractions were pooled. Ammonium sulphate was added to a final concentration of 1 M. The protein was loaded onto a Phenyl Sepharose™ 6 Fast Flow column (high sub) (GE Healthcare, Piscataway, N.J., USA) equilibrated in 20 mM Tris pH 7.5 with 1 M ammonium sulfate, and bound proteins were eluted with a linear gradient from 1 to 0.3 M ammonium sulfate. The purified protein was concentrated and buffer exchanged using a tangential flow concentrator (Pall Filtron, Northborough, Mass., USA) equipped with a 10 kDa polyethersulfone membrane (Pall Filtron, Northborough, Mass., USA) into 50 mM sodium acetate pH 5.0 containing 100 mM sodium chloride. Protein concentration was determined using a Microplate BCA™ Protein Assay Kit (Thermo Fisher Scientific, Inc., Waltham, Mass., USA) in which bovine serum albumin was used as a protein standard.

Thermomyces lanuginosus AA9 (GH61) polypeptide (GenSeqP:AZZ14902; SEQ ID NO: 46) was prepared according to WO 2012/113340.

Aspergillus fumigatus AA9 (GH61B) polypeptide variant was prepared according to WO 2012/044835, which is incorporated herein in its entirety. The filtered broth of the Aspergillus fumigatus GH61B variant polypeptide was concentrated and buffer exchanged using a tangential flow concentrator (Pall Filtron, Northborough, Mass., USA) equipped with a 5 kDa polyethersulfone membrane (Pall Filtron, Northborough, Mass., USA) into 20 mM Tris pH 8.0. The buffer-exchanged protein was loaded onto a SUPERDEX® 75 HR 26/60 column (GE Healthcare, Piscataway, N.J., USA) equilibrated with 20 mM Tris-150 mM sodium chloride pH 8.5. Pooled fractions were concentrated and buffer exchanged using a tangential flow concentrator equipped with a 5 kDa polyethersulfone membrane into 20 mM Tris pH 8.0. Protein concentration was determined using a Microplate BCA™ Protein Assay Kit in which bovine serum albumin.

Aspergillus aculeatus beta-glucosidase (GeneSeqP:AUM17214; SEQ ID NO: 89) was prepared according to WO 2012/044835.

CELLIC® HTec3, a hemicellulase preparation, was obtained from Novozymes NS (Bagsvaerd, Denmark).

Thermoascus aurantiacus catalase (GeneSeqP:BAC11005; SEQ ID NO: 90) was prepared according to WO 2012/130120

Myceliophthora thermophila laccase (GeneSeqP:AAW19855; SEQ ID NO: 91) was prepared according to WO 95/033836.

Polyporus pinsitus laccase (GeneSeqP:AAR90721; SEQ ID NO: 92) was prepared according to WO 96/000290.

Soybean peroxidase (GeneSeqP:AZY11808; SEQ ID NO: 93) was prepared according to WO 2012/098246.

Coprinus cinereus peroxidase (GeneSeqP:AAR75422; SEQ ID NO: 94) was obtained from Novozymes NS as NZ51004. Coprinus cinereus peroxidase was purified as described by WO 1992/016634, and Xu et al., 2003, “Fusion proteins containing Coprinus cinereus peroxidase and the cellulose-binding domain of Humicola insolens family 45 endoglucanase” in Application of Enzymes to Lignocellulosics (Mansfield, S. D. and Saddler, J. N. eds.) pp. 382-402, American Chemical Society, Washington, D.C. The purification scheme comprised ultrafiltration and anion-exchange chromatography. Cell-free broth of a Coprinus cinereus peroxidase (pH 7.7, 11 mS conductivity) was filtered with Whatman #2 paper and ultrafiltered with a polyethersulfone membrane (30 kDa molecular weight cutoff). The washed and concentrated broth (pH 7.7, 1 mS) was then loaded onto a Q-SEPHAROSE BIG BEAD™ column pre-equilibrated with 5 mM CaCl₂-10 mM Tris-HCl pH 7.6 (Buffer A). The active fraction eluted by 5% Buffer B (Buffer A plus 2 M NaCl) was washed (with 5 mM CaCl₂) to 1 mS, then applied to a MONO-Q™ column (GE Healthcare, Piscataway, N.J., USA) equilibrated with Buffer A. Buffer B was used again for the elution. Fractions were analyzed for peroxidase activity and by SDS-PAGE. Specific peroxidase activity was assayed at 30° C. with 0.1 M sodium phosphate pH 7, 0.9 mM H₂O₂, and 1.7 mM 2,2′-azinobis(3-ethylbenzothiazoline-6-sulfonic acid) (ABTS), by monitoring the absorption increase at 418 nm. A stock concentration of 630 μM peroxidase was used.

TABLE 1
Summary of Enzymes

| Enzyme | Source | Abbreviation |
|---|---|---|
| Endoglucanase V core | Humicola insolens | EG |
| CBH I | Aspergillus fumigatus | AfCBHI |
| CBH II | Aspergillus fumigatus | AfCBHII |
| AA9 (GH61A) | Thermoascus aurantiacus | TaGH61A |
| AA9 | Penicillium sp. (emersonii) | PeGH61A |
| AA9 | Thermomyces lanuginosus | TlGH61 |
| AA9 Variant | Aspergillus fumigatus | AfGH61B-B3 |
| β-Glucosidase | Aspergillus aculeatus | AaBG |
| Hemicellulases | — | CELLIC ® |
| | | HTec3 |
| Peroxidase | Coprinus cinereus | CcP |
| Peroxidase | Soybean | Soy P |
| Catalase | Thermoascus aurantiacus | TaC |
| Laccase | Myceliophthora thermophila | MtL |
| Laccase | Polyporus pinsitus | PpL |

### Example 2

### Preparation of Pretreated Corn Stover

Corn stover was pretreated at the U.S. Department of Energy National Renewable Energy Laboratory (NREL), Golden, Colo., USA, using 5% sulfuric acid (g/g on dry corn stover basis) at 190° C. for 1 minute. The composition and the fraction of insoluble solid (FIS) of the pretreated corn stover (PCS) were determined by following the Standard Analytical Procedures developed by NREL (Sluiter et al., 2008, Determination of Total Solids in Biomass and Total Dissolved Solids in Liquid Process Samples. NREL/TP-510-42621. National Renewable Research Laboratory, Golden, Colo., USA; Sluiter et al., 2008, Determination of structural carbohydrates and lignin in biomass. Laboratory Analytical Procedures. NREL/TP-510-42618. National Renewable Research Laboratory, Golden, Colo., USA; Sluiter et al., 2008, Determination of Total Solids in Biomass and Total Dissolved Solids in Liquid Process Samples. Laboratory Analytical Procedures. NREL/TP-510-42621. National Renewable Research Laboratory, Golden, Colo., USA). The water insoluble solids in the PCS contained 57.6% glucan, 2% xylan, and 29.7% acid insoluble lignin. The fraction of insoluble solids (FIS) of the PCS was 61.3%.

### Example 3

### Enzymatic Hydrolysis of PCS

Batch enzymatic hydrolysis was performed in 50 ml Nalgene polycarbonate centrifuge tubes (Thermo Scientific, Pittsburgh, Pa., USA). PCS was mixed with 50 mM sodium acetate pH 5.0 buffer supplemented with enzymes (cellulase, hemicellulase, AA9 polypeptide, and oxidoreductase(s)), as well as 2.5 mg/liter lactrol to prevent microbial growth. All enzymes used in this study are summarized in Table 1. The final total solid concentration was 20% (w/w on a dry weight basis) unless otherwise specified. The reaction mixtures (20 g) were agitated in a hybridization incubator (Combi-D24, FINEPCR®, Yang-Chung, Seoul, Korea) at 50° C. for 120 hours. At the end of hydrolysis, 600 μl of hydrolysate were transferred to a Costar Spin-X centrifuge filter tube (Cole-Parmer, Vernon Hills, Ill., USA) and filtered through 0.2 μm nylon filters during centrifugation (14,000 rpm, 20 minutes). Each supernatant was acidified with 5 μl of 40% (w/v) sulfuric acid to deactivate residual enzyme activity and then analyzed by high performance liquid chromatography (HPLC) for sugar concentrations.

Sugars released from hydrolysis of PCS were analyzed by HPLC using a 1200 Series LC System (Agilent Technologies Inc., Palo Alto, Calif., USA) equipped with a Rezex ROA-Organic acid H⁺ column (8%) (7.8×300 mm) (Phenomenex Inc., Torrance, Calif., USA), 0.2 μm in line filter, an automated sampler, a gradient pump, and a refractive index detector. The mobile phase used was 5 mM sulfuric acid at a flow rate of 0.9 ml/minute. Monomeric sugars at concentrations of 0, 10, 30, and 50 mg/liter were used as standards.

### Example 4

### Synergistic Effect Between Coprinus cinereus Peroxidase and Thermoascus aurantiacus AA9 (GH61A) Polypeptide

Hydrolysis of PCS was performed as described in Example 3 using a cellulase and hemicellulase mixture composed of 10% Humicola insolens endoglucanase V core (EGV core), 35% Aspergillus fumigatus CBHI (AfCBHI), 35% Aspergillus fumigatus CBHII (AfCBHII), 10% Aspergillus aculeatus beta-glucosidase (AaBG), and 10% hemicellulases (Cellic® HTec3). Total protein dosage of cellulases and hemicellulases were 4 mg/g PCS cellulose. Thermoascus aurantiacus AA9 polypeptide (TaGH61A) and Coprinus cinereus peroxidase (CcP) were dosed at 5-20% and 1.5-3.0%, respectively, of the 4 mg dose above as outlined in Table 2. Samples were taken at 120 hours and analyzed by HPLC as described in Example 3.

TABLE 2
Experimental design: testing the synergy between Coprinus cinereus
peroxidase and T. aurantiacus AA9 polypeptide

| Sample | EGV | | | | Cenllic ® | | |
|---|---|---|---|---|---|---|---|
| ID | core | AfCBHI | AfCBHII | AaBG | HTec3 | TaGH61A | CcP |
| 1 | 10% | 35% | 35% | 10% | 10% | 5% | |
| 2 | 10% | 35% | 35% | 10% | 10% | 10% | |
| 3 | 10% | 35% | 35% | 10% | 10% | 15% | |
| 4 | 10% | 35% | 35% | 10% | 10% | 20% | |
| 5 | 10% | 35% | 35% | 10% | 10% | | 1.5% |
| 6 | 10% | 35% | 35% | 10% | 10% | | 3% |
| 7 | 10% | 35% | 35% | 10% | 10% | 5% | 1.5% |
| 8 | 10% | 35% | 35% | 10% | 10% | 5% | 3% |
| 9 | 10% | 35% | 35% | 10% | 10% | 10% | 1.5% |
| 10 | 10% | 35% | 35% | 10% | 10% | 10% | 3% |
| 11 | 10% | 35% | 35% | 10% | 10% | 15% | 1.5% |
| 12 | 10% | 35% | 35% | 10% | 10% | 15% | 3% |
| 13 | 10% | 35% | 35% | 10% | 10% | 20% | 1.5% |
| 14 | 10% | 35% | 35% | 10% | 10% | 20% | 3% |

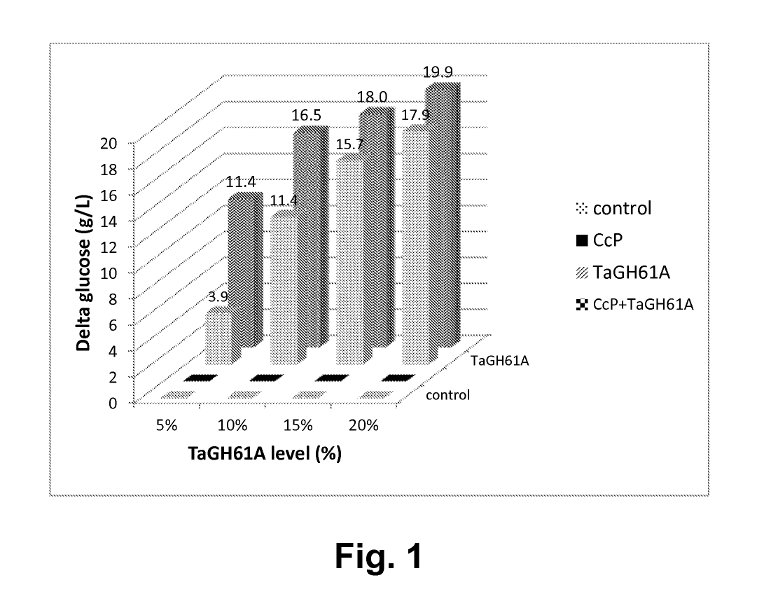

The results as shown in FIG. 1 demonstrated that a synergistic effect existed between the C. cinereus peroxidase and T. aurantiacus AA9 polypeptide. The total glucose yield increased by 11.4-19.9 g/liter when both the C. cinereus peroxidase and T. aurantiacus AA9 polypeptide were dosed together, which was significantly higher than the combination of the boosting effects by the C. cinereus peroxidase alone and the T. aurantiacus AA9 polypeptide alone. The synergistic effect was more significant as the T. aurantiacus AA9 polypeptide level decreased.

### Example 5

### Synergistic Effect Between T. aurantiacus Catalase and T. aurantiacus AA9 (GH61A) Polypeptide

Hydrolysis of PCS was performed as described in Examples 3 and 4 using a cellulase and hemicellulase mixture composed of 10% Humicola insolens endoglucanase V core (EGV core), 35% Aspergillus fumigatus CBHI (AfCBHI), 35% Aspergillus fumigatus CBHII (AfCBHII), 10% Aspergillus aculeatus beta-glucosidase (AaBG), and 10% hemicellulases (Cellic® HTec3). Total protein dosage of cellulases and hemicellulases were 4 mg/g PCS cellulose. The T. aurantiacus AA9 polypeptide (TaGH61A) and T. aurantiacus catalase (TaC) were dosed at 5-20% and 1.5-3%, respectively, of the 4 mg dose as outlined in Table 3. Samples were taken at 120 hours and analyzed by HPLC as described in Example 3.

TABLE 3
Experimental design: Testing the synergy between T. aurantiacus
catalase and T. aurantiacus AA9 polypeptide

| Sample | EGV | | | | Cellic ® | | |
|---|---|---|---|---|---|---|---|
| ID | core | AfCBHI | AfCBHII | AaBG | HTec3 | TaGH61A | TaC |
| 1 | 10% | 35% | 35% | 10% | 10% | 5% | |
| 2 | 10% | 35% | 35% | 10% | 10% | 10% | |
| 3 | 10% | 35% | 35% | 10% | 10% | 15% | |
| 4 | 10% | 35% | 35% | 10% | 10% | 20% | |
| 5 | 10% | 35% | 35% | 10% | 10% | | 1.5% |
| 6 | 10% | 35% | 35% | 10% | 10% | | 3% |
| 7 | 10% | 35% | 35% | 10% | 10% | 5% | 1.5% |
| 8 | 10% | 35% | 35% | 10% | 10% | 5% | 3% |
| 9 | 10% | 35% | 35% | 10% | 10% | 10% | 1.5% |
| 10 | 10% | 35% | 35% | 10% | 10% | 10% | 3% |
| 11 | 10% | 35% | 35% | 10% | 10% | 15% | 1.5% |
| 12 | 10% | 35% | 35% | 10% | 10% | 15% | 3% |
| 13 | 10% | 35% | 35% | 10% | 10% | 20% | 1.5% |
| 14 | 10% | 35% | 35% | 10% | 10% | 20% | 3% |

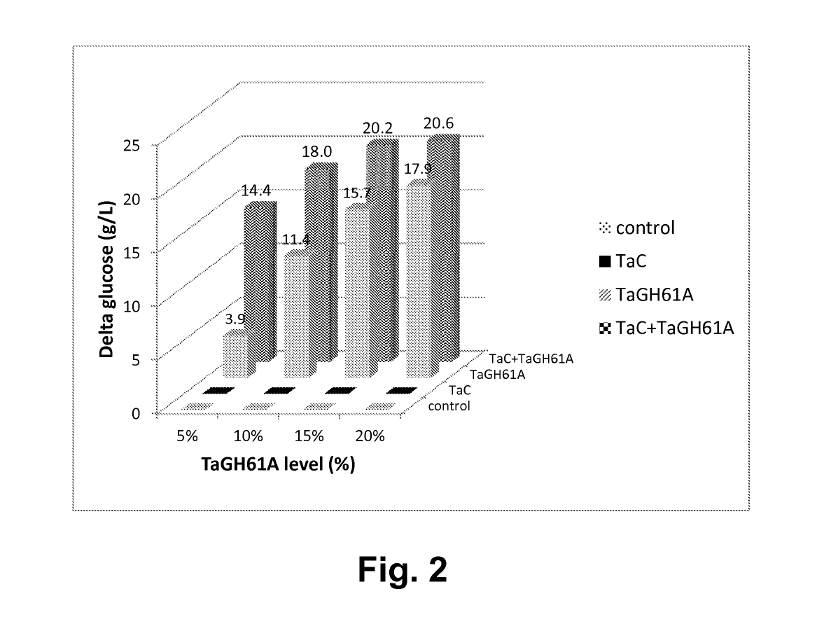

The results as shown in FIG. 2 demonstrated a synergistic effect of the T. aurantiacus catalase and T. aurantiacus AA9 polypeptide together. The total glucose yield increased by 14.4-20.6 g/liter when both the T. aurantiacus catalase and T. aurantiacus AA9 polypeptide were dosed together, which was significantly higher than the combination of the boosting effects by the T. aurantiacus catalase alone and the T. aurantiacus AA9 polypeptide alone. The synergistic effect was more significant as the T. aurantiacus AA9 polypeptide level decreased.

### Example 6

### Synergistic Effect Between M. thermophila Laccase and T. aurantiacus AA9 (GH61A) Polypeptide

Hydrolysis of PCS was performed as described in Examples 3 and 4 using a cellulase and hemicellulase mixture composed of 10% Humicola insolens endoglucanase V core (EGV core), 35% Aspergillus fumigatus CBHI (AfCBHI), 35% Aspergillus fumigatus CBHII (AfCBHII), 10% Aspergillus aculeatus beta-glucosidase (AaBG), and 10% hemicellulases (Cellic® HTec3). Total protein dosage of cellulases and hemicellulases were 4 mg/g PCS cellulose. The T. aurantiacus AA9 polypeptide (TaGH61A) and M. thermophila laccase (MtL) were dosed at 5-20% and 12.5-25 μg/g glucan (0.32-0.63%), respectively, of the 4 mg dose as outlined in Table 4. Samples were taken at 120 hours and analyzed by a HPLC as described in Example 3.

TABLE 4
Experimental design: testing the synergy between M. thermophila laccase
and T. aurantiacus AA9 polypeptide

| Sample ID | EGV core | AfCBHI | AfCBHII | AaBG | Cellic ® HTec3 | TaGH61A | MtL |
|---|---|---|---|---|---|---|---|
| 1 | 10% | 35% | 35% | 10% | 10% | 5% | |
| 2 | 10% | 35% | 35% | 10% | 10% | 10% | |
| 3 | 10% | 35% | 35% | 10% | 10% | 15% | |
| 4 | 10% | 35% | 35% | 10% | 10% | 20% | |
| 5 | 10% | 35% | 35% | 10% | 10% | | 0.32% |
| 6 | 10% | 35% | 35% | 10% | 10% | | 0.63% |
| 7 | 10% | 35% | 35% | 10% | 10% | 5% | 0.32% |
| 8 | 10% | 35% | 35% | 10% | 10% | 5% | 0.63% |
| 9 | 10% | 35% | 35% | 10% | 10% | 10% | 0.32% |
| 10 | 10% | 35% | 35% | 10% | 10% | 10% | 0.63% |
| 11 | 10% | 35% | 35% | 10% | 10% | 15% | 0.32% |
| 12 | 10% | 35% | 35% | 10% | 10% | 15% | 0.63% |
| 13 | 10% | 35% | 35% | 10% | 10% | 20% | 0.32% |
| 14 | 10% | 35% | 35% | 10% | 10% | 20% | 0.63% |

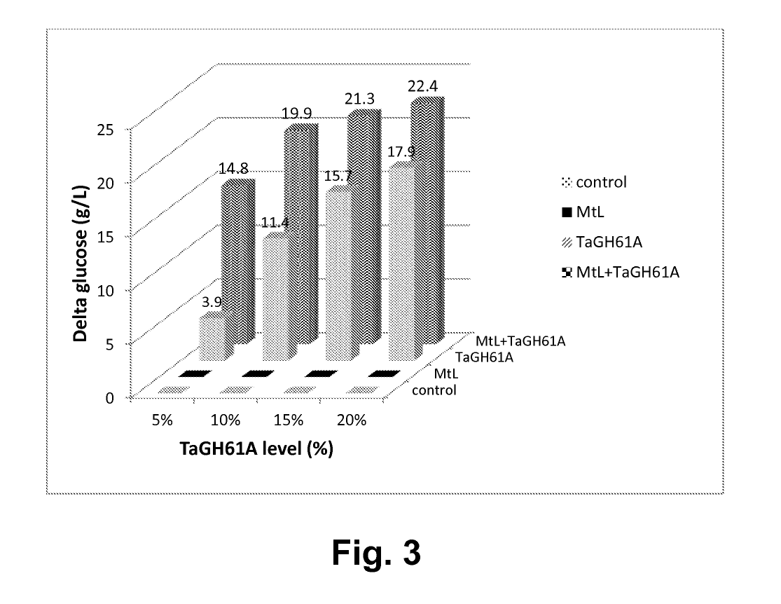

The results as shown in FIG. 3 demonstrated a synergistic effect of the M. thermophila laccase and T. aurantiacus AA9 polypeptide together. The total glucose yield increased by 14.8-22.4 g/liter when both the M. thermophila laccase and T. aurantiacus AA9 polypeptide were dosed together, which was significantly higher than the combination of the boosting effects by the M. thermophila laccase alone and the T. aurantiacus AA9 polypeptide alone. The synergistic effect was more significant as the T. aurantiacus AA9 polypeptide level decreased. The enzyme dosage requirement for the M. thermophila laccase was 5× lower than that for the C. cinereus peroxidase or T. aurantiacus catalase.

### Example 7

### Synergistic Effect Between Various AA9 (GH61) Polypeptides and Oxidoreductases

Hydrolysis of PCS was performed as described in Example 3. The experimental design is shown in Table 5. The numbers represent percentages of each component based on the total protein dosage of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase (AaBG), and hemicellulases (Cellic® HTec3), which was 4 mg/g PCS cellulose. The AA9 polypeptide (T. aurantiacus AA9 polypeptide [TaGH61A], Penicillium sp. AA9 polypeptide [PeGH61A], or A. fumigatus AA9 polypeptide variant [AfGH61B-B3]), M. thermophila laccase (MtL), T. aurantiacus catalase (TaC), or their combinations, were dosed at the percentages shown in Table 5 of the 4 mg dose. Samples were taken at 72 and 120 hours and analyzed by HPLC as described in Example 3.

TABLE 5
Experimental design: Synergistic effect between various AA9 polypeptides and
oxidoreductases

| Sample | | Aa | Cellic ® | | | AfGH61B- | | |
|---|---|---|---|---|---|---|---|---|
| ID | Cellulases | BG | HTec3 | TaGH61A | PeGH61A | B3 | MtL | TaC |
| 1 | 85% | 5% | 10% | | | | | |
| 2 | 85% | 5% | 10% | | | | 0.63% | |
| 3 | 85% | 5% | 10% | | | | | 3.0% |
| 4 | 85% | 5% | 10% | | | | 0.31% | 1.5% |
| 5 | 85% | 5% | 10% | 5% | | | | |
| 6 | 85% | 5% | 10% | 5% | | | 0.63% | |
| 7 | 85% | 5% | 10% | 5% | | | | 3.0% |
| 8 | 85% | 5% | 10% | 5% | | | 0.31% | 1.5% |
| 9 | 85% | 5% | 10% | | 5% | | | |
| 10 | 85% | 5% | 10% | | 5% | | 0.63% | |
| 11 | 85% | 5% | 10% | | 5% | | | 3.0% |
| 12 | 85% | 5% | 10% | | 5% | | 0.31% | 1.5% |
| 13 | 85% | 5% | 10% | | | 5% | | |
| 14 | 85% | 5% | 10% | | | 5% | 0.63% | |
| 15 | 85% | 5% | 10% | | | 5% | | 3.0% |
| 16 | 85% | 5% | 10% | | | 5% | 0.31% | 1.5% |

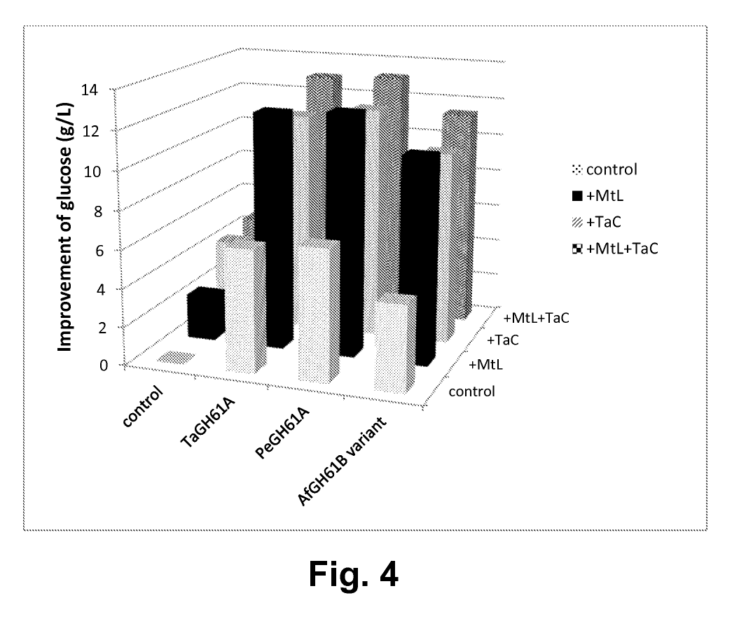

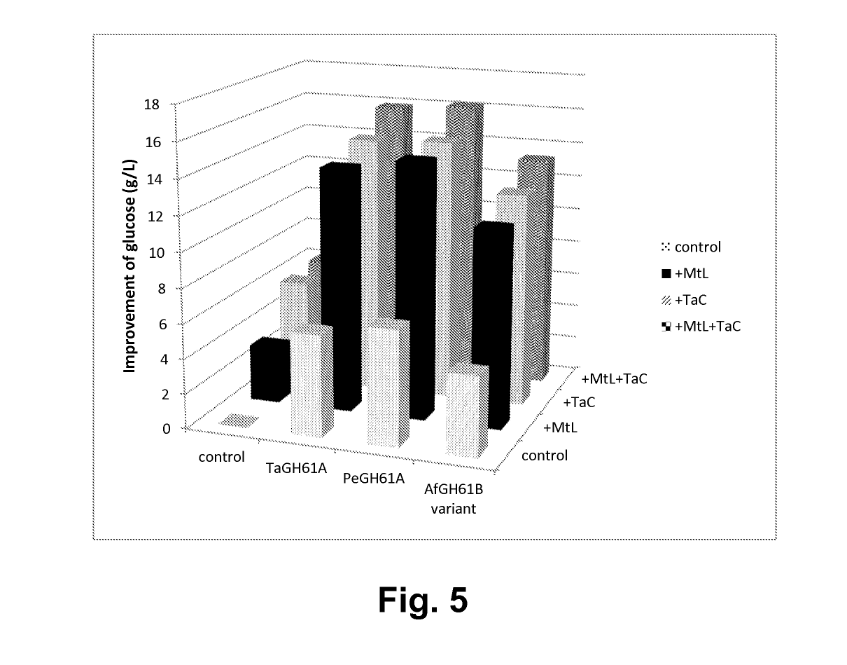

FIGS. 4 and 5 show the improvement of glucose yield from each treatment compared to the control, which was from PCS hydrolyzed with an enzyme composition composed of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase, and hemicellulases (Cellic® HTec3) at 4 mg/g PCS cellulose. Each of the AA9 polypeptide components improved PCS hydrolysis by 4-7 g/liter. The improvement from the M. thermophila laccase, T. aurantiacus catalase, and the combination of the M. thermophila and T. aurantiacus catalase were 2-4 g/liter. A synergistic effect existed between the oxidoreductases and the AA9 polypeptides. The total glucose yield increased by 10-13 g/liter (72 hours) and 11-16 g/liter (120 hours) when both oxidoreductases and AA9 polypeptide were dosed together, which was significantly higher than the combination of the boosting effects by oxidoreductases alone and AA9 polypeptide alone. The combination of the M. thermophila laccase and T. aurantiacus catalase at a 1:1 ratio (based on enzyme protein) showed a slightly better synergistic effect with the AA9 polypeptides than the oxidoreductases dosed individually.

### Example 8

### Synergistic Effect Between Thermomyces lanuginosus AA9 (GH61) Polypeptide and Oxidoreductases

Hydrolysis of PCS was performed as described in Example 3. The experimental design is shown in Table 6. The numbers represent percentages of each component based on the total protein dosage of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase (AaBG), and hemicellulases (Cellic® HTec3), which was 4 mg/g PCS cellulose. The Thermomyces lanuginosus AA9 polypeptide (TIGH61), M. thermophila laccase (MtL), T. aurantiacus catalase (TaC), or their combinations were dosed at the percentages shown in Table 6 of the 4 mg dose. Samples were taken at 72 and 120 hours and analyzed by HPLC as described in Example 3.

TABLE 6
Experimental design: Synergistic effect between various
oxidoreductases and T. aurantiacus AA9 polypeptide

| Sample | | Aa | Cellic ® | | | |
|---|---|---|---|---|---|---|
| ID | Cellulases | BG | HTec3 | TIGH61 | MtL | TaC |
| 1 | 85% | 5% | 10% | | | |
| 2 | 85% | 5% | 10% | | 0.63% | |
| 3 | 85% | 5% | 10% | | | 3.0% |
| 4 | 85% | 5% | 10% | | 0.31% | 1.5% |
| 17 | 85% | 5% | 10% | 2.5% | | |
| 18 | 85% | 5% | 10% | 2.5% | 0.63% | |
| 19 | 85% | 5% | 10% | 2.5% | | 3.0% |
| 20 | 85% | 5% | 10% | 2.5% | 0.31% | 1.5% |

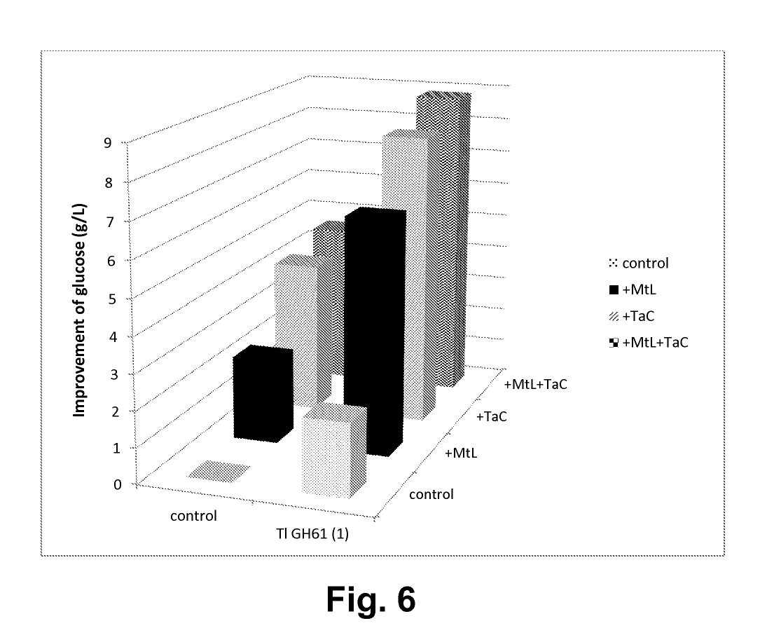

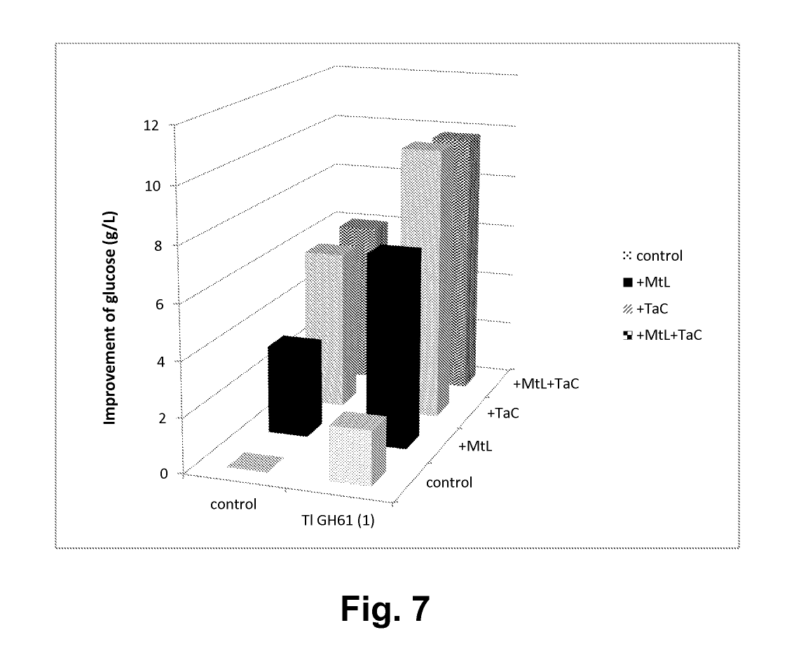

FIGS. 6 and 7 show the improvement of glucose yield from each treatment compared to a control. The control was PCS hydrolyzed with an enzyme composition composed of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase, and hemicellulases (Cellic® HTec3) at 4 mg/g PCS cellulose. The T. lanuginosus AA9 polypeptide at a 2.5% level improved PCS hydrolysis by approximately 2 g/liter. The improvement from the M. thermophila laccase, T. aurantiacus catalase, and the combination of the M. thermophila laccase and T. aurantiacus catalase were 2-4 g/liter. A synergistic effect existed between the oxidoreductases and the T. lanuginosus AA9 polypeptide. The total glucose yield increased by 6-9 g/liter (72 hours) and 7-10 g/liter (120 hours) when both oxidoreductases and the T. lanuginosus AA9 polypeptide were dosed together, which was significantly higher than the combination of the boosting effects by the oxidoreductases alone or the T. lanuginosus AA9 polypeptide alone. The combination of the M. thermophila laccase and T. lanuginosus catalase at a 1:1 ratio (based on enzyme protein) showed a similar synergistic effect with the T. lanuginosus AA9 polypeptide than the oxidoreductases dosed individually.

### Example 9

### Synergistic Effect Between Thermoascus aurantiacus AA9 (GH61A) Polypeptide and Multiple Oxidoreductases

Hydrolysis of PCS was performed as described in Example 3. The experimental design is shown in Table 7. The numbers represent percentages of each component based on the total protein dosage of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase (AaBG), and hemicellulases (Cellic® HTec3), which was 4 mg/g PCS cellulose. The T. aurantiacus AA9 polypeptide (TaGH61A; 200 μg/g glucan), M. thermophila laccase (MtL; 6.25-12.5 μg/g glucan), P. pinsitus laccase (PpL; 3-8.6 μg/g glucan), soybean peroxidase (Soy P; 40-160 μg/g glucan), C. cinereus peroxidase (CcP; 30-60 μg/g glucan), T. aurantiacus catalase (TaC; 30-60 μg/g glucan), or their combinations, were dosed at the percentages shown in Table 7 of the 4 mg dose. Samples were taken at 72 and 120 hours and analyzed by HPLC as described in Example 3.

TABLE 7
Experimental design: Synergistic effect between multiple oxidoreductases
and T. aurantiacus AA9 polypeptide

| Sample | | Aa | Cellic ® | | | | | | |
|---|---|---|---|---|---|---|---|---|---|
| ID | Cellulase | BG | HTec3 | TaGH61A | MtL | PpL | TaC | Soy P | CcP |
| 1 | 85% | 5% | 10% | | | | | | |
| 2 | 85% | 5% | 10% | | | 0.11% | | | |
| 3 | 85% | 5% | 10% | | | 0.22% | | | |
| 4 | 85% | 5% | 10% | 5% | | 0.11% | | | |
| 5 | 85% | 5% | 10% | 5% | | 0.22% | | | |
| 6 | 85% | 5% | 10% | | | | | | |
| 7 | 85% | 5% | 10% | | | | | | |
| 8 | 85% | 5% | 10% | 5% | | | | | |
| 9 | 85% | 5% | 10% | 5% | | | | | |
| 10 | 85% | 5% | 10% | | | | | 2% | |
| 11 | 85% | 5% | 10% | | | | | 4% | |
| 12 | 85% | 5% | 10% | 5% | | | | 2% | |
| 13 | 85% | 5% | 10% | 5% | | | | 4% | |
| 14 | 85% | 5% | 10% | | 0.31% | 0.075% | | | |
| 15 | 85% | 5% | 10% | 5% | 0.31% | 0.075% | | | |
| 16 | 85% | 5% | 10% | | | | 1.5% | | |
| 17 | 85% | 5% | 10% | 5% | | | 1.5% | | |
| 18 | 85% | 5% | 10% | | | | | 1% | 1.5% |
| 19 | 85% | 5% | 10% | 5% | | | | 1% | 1.5% |
| 20 | 85% | 5% | 10% | | 0.31% | | 1.5% | | |
| 21 | 85% | 5% | 10% | 5% | 0.31% | | 1.5% | | |
| 22 | 85% | 5% | 10% | | 0.31% | | | | 1.5% |
| 23 | 85% | 5% | 10% | 5% | 0.31% | | | | 1.5% |
| 24 | 85% | 5% | 10% | | | | 1.5% | | 1.5% |
| 25 | 85% | 5% | 10% | 5% | | | 1.5% | | 1.5% |
| 26 | 85% | 5% | 10% | | 0.16% | | 0.75% | | 0.75% |
| 27 | 85% | 5% | 10% | 5% | 0.16% | | 0.75% | | 0.75% |
| 28 | 85% | 5% | 10% | 5% | | | | | |

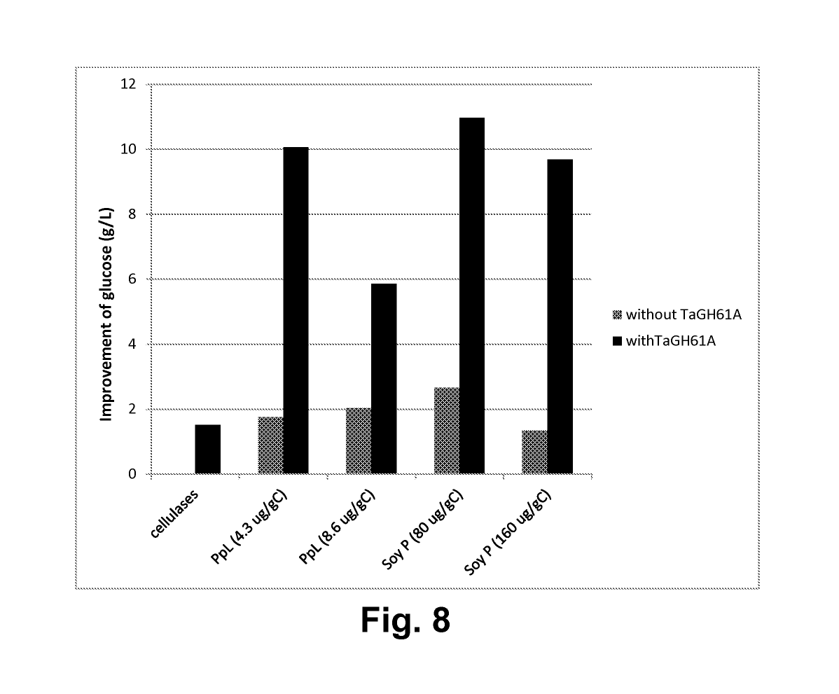

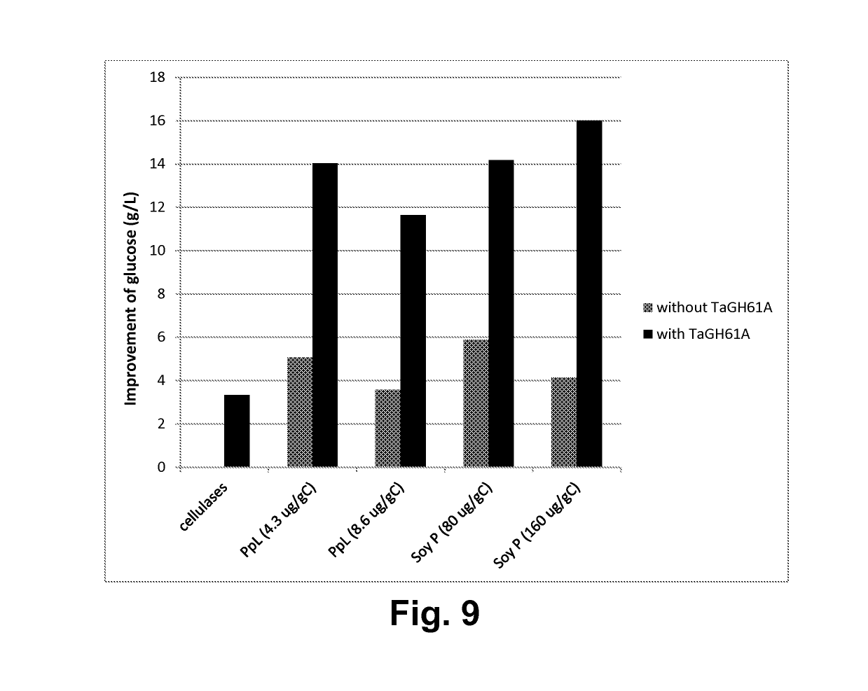

FIGS. 8 and 9 show the synergistic effect between an individual oxidoreductase and T. aurantiacus AA9 polypeptide. The control was PCS hydrolyzed with an enzyme composition composed of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase, and hemicellulases (Cellic® HTec3) at 4 mg/g PCS cellulose. The T. aurantiacus AA9 polypeptide at a 5% level improved PCS hydrolysis by approximately 1.7 and 3.3 g/liter after 72 and 120 hours, respectively. In the absence of the T. aurantiacus AA9 polypeptide, the improvement from the P. pinsitus laccase or Soybean peroxidase was 0.1-2.7 and 1.2-5.9 g/liter after 72 and 120 hours, respectively. In the presence of 5% T. aurantiacus AA9 polypeptide, a synergistic effect existed between an individual oxidoreductase and the T. aurantiacus AA9 polypeptide. The total glucose yield increased by 5-11 g/liter (72 hours) and 4.3-16 g/liter (120 hours), which was significantly higher than the combination of the boosting effects by the individual oxidoreductase alone or the T. aurantiacus AA9 polypeptide alone.

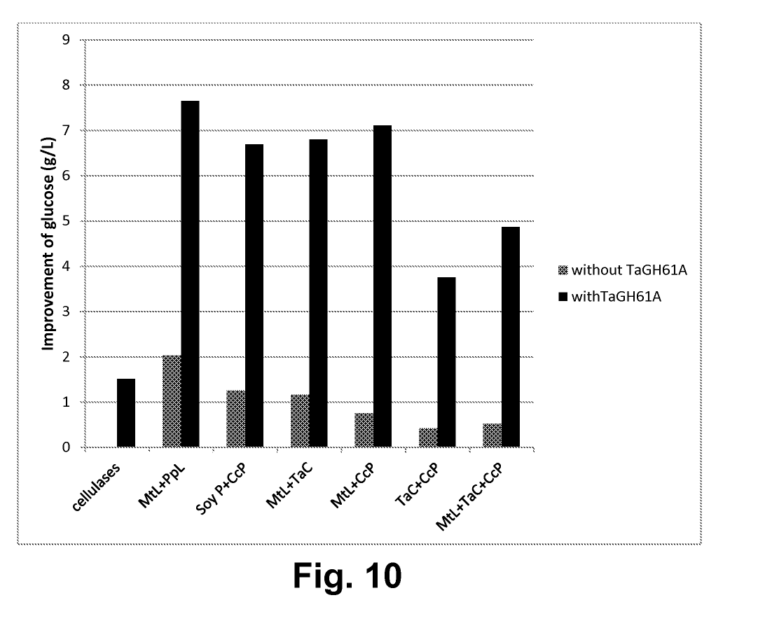

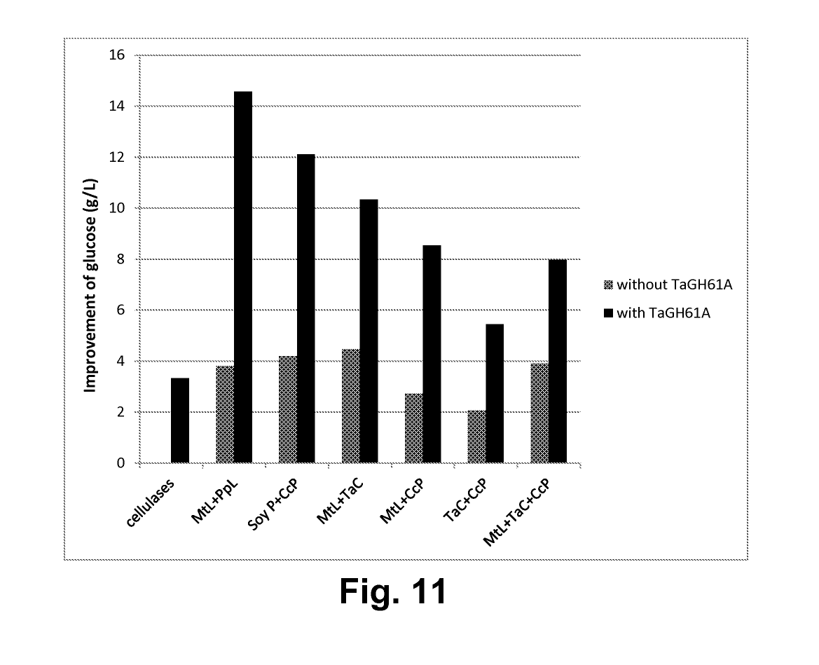

FIGS. 10 and 11 show the synergistic effect between multiple oxidoreductases and the T. aurantiacus AA9 polypeptide. The control was PCS hydrolyzed with an enzyme composition composed of cellulases (Trichoderma reesei cellulase with Aspergillus fumigatus cellobiohydrolase I and Aspergillus fumigatus cellobiohydrolase II replacing the T. reesei cellobiohydrolase I and cellobiohydrolase II), A. aculeatus beta-glucosidase, and hemicellulases (Cellic® HTec3) at 4 mg/g PCS cellulose. The T. aurantiacus AA9 polypeptide at a 5% level improved PCS hydrolysis by approximately 1.7 and 3.3 g/liter after 72 and 120 hours, respectively. In the absence of the T. aurantiacus AA9 polypeptide, the improvement from two or more oxidoreductases were 0.4-2.0 and 2.1-4.5 g/liter after 72 and 120 hours, respectively. In the presence of 5% T. aurantiacus AA9 polypeptide, a synergistic effect existed between the combination of two or more oxidoreductases and the T. aurantiacus AA9 polypeptide. The total glucose yield increased by 3.8-7.6 g/liter (72 hours) and 2.1-14.6 g/liter (120 hours), which is significantly higher than the combination of the boosting effects by the multiple oxidoreductases alone or the T. aurantiacus AA9 polypeptide alone.

The present invention is further described by the following numbered paragraphs:

[1] A process for degrading a cellulosic material, comprising: treating the cellulosic material with an enzyme composition in the presence of a combination of an AA9 polypeptide and one or more oxidoreductases selected from the group consisting of a catalase, a laccase, and a peroxidase.

[2] The process of paragraph 1, wherein the combination of the AA9 polypeptide and the one or more oxidoreductases is the AA9 polypeptide and one oxidoreductase.

[3] The process of paragraph 2, wherein the protein content of the combination of the AA9 polypeptide and the one oxidoreductase is in the range of about 0.5% to about 25% of total protein.

[4] The process of paragraph 2 or 3, wherein the one oxidoreductase is a catalase, laccase, or peroxidase.

[5] The process of paragraph 4, wherein the protein ratio of the AA9 polypeptide to the catalase is in the range of about 0.5:1 to about 15:1, the protein ratio of the AA9 polypeptide to the laccase is in the range of about 3:1 to about 150:1, and the protein ratio of the AA9 polypeptide to the peroxidase is in the range of about 0.5:1 to about 15:1.

[6] The process of paragraph 1, wherein the combination of the AA9 polypeptide and the one or more oxidoreductases is the AA9 polypeptide and two oxidoreductases.

[7] The process of paragraph 6, wherein the protein content of the combination of the AA9 polypeptide and the two oxidoreductase is in the range of about 0.5% to about 25% of total protein.

[8] The process of paragraph 6 or 7, wherein the two oxidoreductases are independently selected from the group of catalases, laccases, and peroxidases.

[9] The process of paragraph 8, wherein the two oxidoreductases are a catalase and a laccase.

[10] The process of paragraph 8, wherein the two oxidoreductases are a catalase and a peroxidase.

[11] The process of paragraph 8, wherein the two oxidoreductases are a laccase and a peroxidase.

[12] The process of paragraph 8, wherein the two oxidoreductases are two catalases.

[13] The process of paragraph 8, wherein the two oxidoreductases are two laccases.

[14] The process of paragraph 8, wherein the two oxidoreductases are two peroxidases.

[15] The process of any of paragraphs 8-14, wherein the protein ratio of the AA9 polypeptide to the catalase is in the range of about 1:1 to about 30:1, the protein ratio of the AA9 polypeptide to the laccase is in the range of about 6:1 to about 300:1, and the protein ratio of the AA9 polypeptide to the peroxidase is in the range of about 1:1 to about 30:1.

[16] The process of paragraph 1, wherein the combination of the AA9 polypeptide and the one or more oxidoreductases is the AA9 polypeptide and three oxidoreductases.

[17] The process of paragraph 10, wherein the protein content of the combination of the AA9 polypeptide and the three oxidoreductases is in the range of about 0.5% to about 25% of total protein.

[18] The process of paragraph 10 or 11, wherein the three oxidoreductases are independently selected from the group of catalases, laccases, and peroxidases.

[19] The process of paragraph 18, wherein the three oxidoreductases are a catalase, a laccase, and a peroxidase.

[20] The process of paragraph 18, wherein the three oxidoreductases are a laccase and two catalases.

[21] The process of paragraph 18, wherein the three oxidoreductases are a peroxidase and two catalases.

[22] The process of paragraph 18, wherein the three oxidoreductases are a catalase and two laccases.

[23] The process of paragraph 18, wherein the three oxidoreductases are a peroxidase and two laccases.

[24] The process of paragraph 18, wherein the three oxidoreductases are a catalase and two peroxidases.

[25] The process of paragraph 18, wherein the three oxidoreductases are a laccase and two peroxidases.

[26] The process of paragraph 18, wherein the three oxidoreductases are three catalases.

[27] The process of paragraph 18, wherein the three oxidoreductases are three laccases.

[28] The process of paragraph 18, wherein the three oxidoreductases are three peroxidases.

[29] The process of any of paragraphs 18-28, wherein the protein ratio of the AA9 polypeptide to the catalase is in the range of about 1:1 to about 30:1, the protein ratio of the AA9 polypeptide to the laccase is in the range of about 6:1 to about 300:1, and the protein ratio of the AA9 polypeptide to the peroxidase is in the range of about 1:1 to about 30:1.

[30] The process of any of paragraphs 1-29, wherein the cellulosic material is pretreated.

[31] The process of any of paragraphs 1-30, wherein the enzyme composition comprises one or more enzymes selected from the group consisting of a cellulase, a hemicellulase, an esterase, an expansin, a ligninolytic enzyme, a pectinase, a protease, and a swollenin.

[32] The process of paragraph 31, wherein the cellulase is one or more enzymes selected from the group consisting of an endoglucanase, a cellobiohydrolase, and a beta-glucosidase.

[33] The process of paragraph 31, wherein the hemicellulase is one or more enzymes selected from the group consisting of a xylanase, an acetylxylan esterase, a feruloyl esterase, an arabinofuranosidase, a xylosidase, and a glucuronidase.

[34] The process of any of paragraphs 1-30, wherein the enzyme composition comprises an endoglucanase, a cellobiohydrolase, and a beta-glucosidase.

[35] The process of any of paragraphs 1-30, wherein the enzyme composition comprises an endoglucanase, a cellobiohydrolase, a beta-glucosidase, a xylanase, and a beta-xylosidase.

[36] The process of any of paragraphs 1-35, further comprising recovering the degraded cellulosic material.

[37] The process of paragraph 36, wherein the degraded cellulosic material is a sugar.

[38] The process of paragraph 37, wherein the sugar is selected from the group consisting of glucose, xylose, mannose, galactose, and arabinose.

[39] A process for producing a fermentation product, comprising: (a) saccharifying a cellulosic material with an enzyme composition in the presence of a combination of an AA9 polypeptide and one or more oxidoreductases selected from the group consisting of a catalase, a laccase, and a peroxidase; (b) fermenting the saccharified cellulosic material with one or more fermenting microorganisms to produce the fermentation product; and (c) recovering the fermentation product from the fermentation.

[40] The process of paragraph 39, wherein the combination of the AA9 polypeptide and the one or more oxidoreductases is the AA9 polypeptide and one oxidoreductase.

[41] The process of paragraph 40, wherein the protein content of the combination of the AA9 polypeptide and the one oxidoreductase is in the range of about 0.5% to about 25% of total protein.

[42] The process of paragraph 40 or 41, wherein the one oxidoreductase is a catalase, a laccase, or a peroxidase.

[43] The process of paragraph 42, wherein the protein ratio of the AA9 polypeptide to the catalase is in the range of about 0.5:1 to about 15:1, the protein ratio of the AA9 polypeptide to the laccase is in the range of about 3:1 to about 150:1, and the protein ratio of the AA9 polypeptide to the peroxidase is in the range of about 0.5:1 to about 15:1.

[44] The process of paragraph 39, wherein the combination of the AA9 polypeptide and the one or more oxidoreductases is the AA9 polypeptide and two oxidoreductases.

[45] The process of paragraph 44, wherein the protein content of the combination of the AA9 polypeptide and the two oxidoreductases is in the range of about 0.5% to about 25% of total protein.

[46] The process of paragraph 44 or 45, wherein the two oxidoreductases are independently selected from the group of catalases, laccases, and peroxidases.

[47] The process of paragraph 46, wherein the two oxidoreductases are a catalase and a laccase.

[48] The process of paragraph 46 wherein the two oxidoreductases are a catalase and a peroxidase.

[49] The process of paragraph 46, wherein the two oxidoreductases are a laccase and a peroxidase.

[50] The process of paragraph 46, wherein the two oxidoreductases are two catalases.

[51] The process of paragraph 46, wherein the two oxidoreductases are two laccases.

[52] The process of paragraph 46, wherein the two oxidoreductases are two peroxidases.

[53] The process of any of paragraphs 46-52, wherein the protein ratio of the AA9 polypeptide to the catalase is in the range of about 1:1 to about 30:1, the protein ratio of the AA9 polypeptide to the laccase is in the range of about 6:1 to about 300:1, and the protein ratio of the AA9 polypeptide to the peroxidase is in the range of about 1:1 to about 30:1.

[54] The process of paragraph 39, wherein the combination of the AA9 polypeptide and the one or more oxidoreductases is the AA9 polypeptide and three oxidoreductases.

[55] The process of paragraph 54, wherein the protein content of the combination of the AA9 polypeptide and the three oxidoreductases is in the range of about 0.5% to about 25% of total protein.

[56] The process of paragraph 54 or 55, wherein the three oxidoreductases are independently selected from the group of catalases, laccases, and peroxidases.

[57] The process of paragraph 56, wherein the three oxidoreductases are a catalase, a laccase, and a peroxidase.

[58] The process of paragraph 56, wherein the three oxidoreductases are a laccase and two catalases.

[59] The process of paragraph 56, wherein the three oxidoreductases are a peroxidase and two catalases.

[60] The process of paragraph 56, wherein the three oxidoreductases are a catalase and two laccases.

[61] The process of paragraph 56, wherein the three oxidoreductases are a peroxidase and two laccases.

[62] The process of paragraph 56, wherein the three oxidoreductases are a catalase and two peroxidases.

[63] The process of paragraph 56, wherein the three oxidoreductases are a laccase and two peroxidases.

[64] The process of paragraph 56, wherein the three oxidoreductases are three catalases.

[65] The process of paragraph 56, wherein the three oxidoreductases are three laccases.

[66] The process of paragraph 56, wherein the three oxidoreductases are three peroxidases.

[67] The process of any of paragraphs 56-66, wherein the protein ratio of the AA9 polypeptide to the catalase is in the range of about 1:1 to about 30:1, the protein ratio of the

AA9 polypeptide to the laccase is in the range of about 6:1 to about 300:1, and the protein ratio of the AA9 polypeptide to the peroxidase is in the range of about 1:1 to about 30:1.

[68] The process of any of paragraphs 39-67, wherein the cellulosic material is pretreated.

[69] The process of any of paragraphs 39-68, wherein the enzyme composition comprises the enzyme composition comprises one or more enzymes selected from the group consisting of a cellulase, a hemicellulase, an esterase, an expansin, a ligninolytic enzyme, a pectinase, a protease, and a swollenin.

[70] The process of paragraph 69, wherein the cellulase is one or more enzymes selected from the group consisting of an endoglucanase, a cellobiohydrolase, and a beta-glucosidase.

[71] The process of paragraph 69, wherein the hemicellulase is one or more enzymes selected from the group consisting of a xylanase, an acetylxylan esterase, a feruloyl esterase, an arabinofuranosidase, a xylosidase, and a glucuronidase.

[72] The process of any of paragraphs 39-68, wherein the enzyme composition comprises an endoglucanase, a cellobiohydrolase, and a beta-glucosidase.

[73] The process of any of paragraphs 39-68, wherein the enzyme composition comprises an endoglucanase, a cellobiohydrolase, a beta-glucosidase, a xylanase, and a beta-xylosidase.

[74] The process of any of paragraphs 39-73, wherein steps (a) and (b) are performed simultaneously in a simultaneous saccharification and fermentation.

[75] The process of any of paragraphs 39-74, wherein the fermentation product is an alcohol, an alkane, a cycloalkane, an alkene, an amino acid, a gas, isoprene, a ketone, an organic acid, or polyketide.

[76] A process of fermenting a cellulosic material, comprising: fermenting the cellulosic material with one or more fermenting microorganisms, wherein the cellulosic material is saccharified with an enzyme composition in the presence of a combination of an AA9 polypeptide and one or more oxidoreductases selected from the group consisting of a catalase, a laccase, and a peroxidase.

[77] The process of paragraph 76, wherein the combination of the AA9 polypeptide and the one or more oxidoreductases is the AA9 polypeptide and one oxidoreductase.

[78] The process of paragraph 77, wherein the protein content of the combination of the AA9 polypeptide and the one oxidoreductase is in the range of about 0.5% to about 25% of total protein.

[79] The process of paragraph 77 or 78, wherein the one oxidoreductase is a catalase, a laccase, or a peroxidase.

[80] The process of paragraph 79, wherein the protein ratio of the AA9 polypeptide to the catalase is in the range of about 0.5:1 to about 15:1, the protein ratio of the AA9 polypeptide to the laccase is in the range of about 3:1 to about 150:1, and the protein ratio of the AA9 polypeptide to the peroxidase is in the range of about 0.5:1 to about 15:1.

[81] The process of paragraph 76, wherein the combination of the AA9 polypeptide and the one or more oxidoreductases is the AA9 polypeptide and two oxidoreductases.

[82] The process of paragraph 81, wherein the protein content of the combination of the AA9 polypeptide and the two oxidoreductases is in the range of about 0.5% to about 25% of total protein.

[83] The process of paragraph 81 or 82, wherein the two oxidoreductases are independently selected from the group of catalases, laccases, and peroxidases.

[84] The process of paragraph 83, wherein the two oxidoreductases are a catalase and a laccase.

[85] The process of paragraph 83, wherein the two oxidoreductases are a catalase and a peroxidase.

[86] The process of paragraph 83, wherein the two oxidoreductases are a laccase and a peroxidase.

[87] The process of paragraph 83, wherein the two oxidoreductases are two catalases.

[88] The process of paragraph 83, wherein the two oxidoreductases are two laccases.

[89] The process of paragraph 83, wherein the two oxidoreductases are two peroxidases.

[90] The process of any of paragraphs 83-89, wherein the protein ratio of the AA9 polypeptide to the catalase is in the range of about 1:1 to about 30:1, the protein ratio of the AA9 polypeptide to the laccase is in the range of about 6:1 to about 300:1, and the protein ratio of the AA9 polypeptide to the peroxidase is in the range of about 1:1 to about 30:1.

[91] The process of paragraph 76, wherein the combination of the AA9 polypeptide and the one or more oxidoreductases is the AA9 polypeptide and three oxidoreductases.

[92] The process of paragraph 91, wherein the protein content of the combination of the AA9 polypeptide and the three oxidoreductases is in the range of about 0.5% to about 25% of total protein.

[93] The process of paragraph 91 or 92, wherein the three oxidoreductases are independently selected from the group of catalases, laccases, and peroxidases.

[94] The process of paragraph 93, wherein the three oxidoreductases are a catalase, a laccase, and a peroxidase.

[95] The process of paragraph 93, wherein the three oxidoreductases are a laccase and two catalases.

[96] The process of paragraph 93, wherein the three oxidoreductases are a peroxidase and two catalases.

[97] The process of paragraph 93, wherein the three oxidoreductases are a catalase and two laccases.

[98] The process of paragraph 93, wherein the three oxidoreductases are a peroxidase and two laccases.

[99] The process of paragraph 93, wherein the three oxidoreductases are a catalase and two peroxidases.

[100] The process of paragraph 93, wherein the three oxidoreductases are a laccase and two peroxidases.

[101] The process of paragraph 93, wherein the three oxidoreductases are three catalases.

[102] The process of paragraph 93, wherein the three oxidoreductases are three laccases.

[103] The process of paragraph 93, wherein the three oxidoreductases are three peroxidases.

[104] The process of any of paragraphs 93-103, wherein the protein ratio of the AA9 polypeptide to the catalase is in the range of about 1:1 to about 30:1, the protein ratio of the AA9 polypeptide to the laccase is in the range of about 6:1 to about 300:1, and the protein ratio of the AA9 polypeptide to the peroxidase is in the range of about 1:1 to about 30:1.

[105] The process of any of paragraphs 76-104, wherein the fermenting of the cellulosic material produces a fermentation product.

[106] The process of paragraph 105, further comprising recovering the fermentation product from the fermentation.

[107] The process of paragraph 105 or 106, wherein the fermentation product is an alcohol, an alkane, a cycloalkane, an alkene, an amino acid, a gas, isoprene, a ketone, an organic acid, or polyketide.

[108] The process of any of paragraphs 76-107, wherein the cellulosic material is pretreated before saccharification.

[109] The process of any of paragraphs 76-108, wherein the enzyme composition comprises one or more enzymes selected from the group consisting of a cellulase, a hemicellulase, an esterase, an expansin, a ligninolytic enzyme, a pectinase, a protease, and a swollenin.

[110] The process of paragraph 109, wherein the cellulase is one or more enzymes selected from the group consisting of an endoglucanase, a cellobiohydrolase, and a beta-glucosidase.

[111] The process of paragraph 109, wherein the hemicellulase is one or more enzymes selected from the group consisting of a xylanase, an acetylxylan esterase, a feruloyl esterase, an arabinofuranosidase, a xylosidase, and a glucuronidase.

[112] The process of any of paragraphs 76-108, wherein the enzyme composition comprises an endoglucanase, a cellobiohydrolase, and a beta-glucosidase.

[113] The process of any of paragraphs 76-108, wherein the enzyme composition comprises an endoglucanase, a cellobiohydrolase, a beta-glucosidase, a xylanase, and a beta-xylosidase.

[114] The process of any paragraphs 1-113, wherein the presence of the combination of the AA9 polypeptide and the one or more oxidoreductases synergistically increases the hydrolysis of the cellulosic material by the enzyme composition at least 1.01-fold compared to the AA9 polypeptide alone, the one or more oxidoreductases alone, or absence of the AA9 polypeptide and the one or more oxidoreductases.

[115] The process of any paragraphs 1-114, wherein the combination of the AA9 polypeptide and the one or more oxidoreductases further comprises one or more non-ionic and/or cationic surfactants.

[116] The process of paragraph 115, wherein the amount of the surfactant is in the range of about 0.01% to about 10% w/w on a dry cellulosic material basis.

[117] The process of any paragraphs 1-116, wherein oxygen is added during the degradation or saccharification of the cellulosic material to maintain a concentration of dissolved oxygen in the range of 0.5 to 10% of the saturation level.

[118] The process of paragraph 117, wherein the dissolved oxygen concentration during saccharification is in the range of 0.5-10% of the saturation level, such as 0.5-7%, such as 0.5-5%, such as 0.5-4%, such as 0.5-3%, such as 0.5-2%, such as 1-5%, such as 1-4%, such as 1-3%, such as 1-2%.

[119] The process of paragraph 117, wherein the dissolved oxygen concentration is maintained in the range of 0.5-10% of the saturation level, such as 0.5-7%, such as 0.5-5%, such as 0.5-4%, such as 0.5-3%, such as 0.5-2%, such as 1-5%, such as 1-4%, such as 1-3%, such as 1-2% during at least 25%, such as at least 50% or at least 75% of the saccharification period.

[120] The process of paragraph 117, wherein oxygen is added during the degradation or saccharification of the cellulosic material to maintain a concentration of dissolved oxygen in the range of 0.025 ppm to 0.55 ppm, such as, e.g., 0.05 to 0.165 ppm.

[121] An enzyme composition comprising a combination of an AA9 polypeptide and one or more oxidoreductases selected from the group consisting of a catalase, a laccase, and a peroxidase.

[122] The enzyme composition of paragraph 121, which further comprises one or more enzymes selected from the group consisting of a cellulase, a hemicellulase, an esterase, an expansin, a ligninolytic enzyme, a pectinase, a protease, and a swollenin.

[123] The enzyme composition of paragraph 122, wherein the cellulase is one or more enzymes selected from the group consisting of an endoglucanase, a cellobiohydrolase, and a beta-glucosidase.

[124] The enzyme composition of paragraph 122, wherein the hemicellulase is one or more enzymes selected from the group consisting of a xylanase, an acetylxylan esterase, a feruloyl esterase, an arabinofuranosidase, a xylosidase, and a glucuronidase.

[125] The enzyme composition of paragraph 121, further comprising an endoglucanase, a cellobiohydrolase, and a beta-glucosidase.

[126] The enzyme composition of paragraph 121, further comprising an endoglucanase, a cellobiohydrolase, a beta-glucosidase, a xylanase, and a beta-xylosidase.

[127] The enzyme composition of any of paragraphs 121-126, wherein the combination of the AA9 polypeptide and the one or more oxidoreductases is the AA9 polypeptide and one oxidoreductase.

[128] The enzyme composition of paragraph 127, wherein the one oxidoreductase is a catalase, a laccase, or a peroxidase.

[129] The enzyme composition of any of paragraphs 121-126, wherein the combination of the AA9 polypeptide and the one or more oxidoreductases is the AA9 polypeptide and two oxidoreductases.

[130] The enzyme composition of paragraph 129, wherein the two oxidoreductases are independently selected from the group of catalases, laccases, and peroxidases.

[131] The enzyme composition of paragraph 130, wherein the two oxidoreductases are a catalase and a laccase.

[132] The enzyme composition of paragraph 130, wherein the two oxidoreductases are a catalase and a peroxidase.

[133] The enzyme composition of paragraph 130, wherein the two oxidoreductases are a laccase and a peroxidase.

[134] The enzyme composition of paragraph 130, wherein the two oxidoreductases are two catalases.

[135] The enzyme composition of paragraph 130, wherein the two oxidoreductases are two laccases.

[136] The enzyme composition of paragraph 130, wherein the two oxidoreductases are two peroxidases.

[137] The enzyme composition of any of paragraphs 121-126, wherein the combination of the AA9 polypeptide and the one or more oxidoreductases is the AA9 polypeptide and three oxidoreductases.

[138] The enzyme composition of paragraph 137, wherein the three oxidoreductases are independently selected from the group of catalases, laccases, and peroxidases.

[139] The enzyme composition of paragraph 138, wherein the three oxidoreductases are a catalase, a laccase, and a peroxidase.

[140] The enzyme composition of paragraph 138, wherein the three oxidoreductases are a laccase and two catalases.

[141] The enzyme composition of paragraph 138, wherein the three oxidoreductases are a peroxidase and two catalases.

[142] The enzyme composition of paragraph 138, wherein the three oxidoreductases are a catalase and two laccases.

[143] The enzyme composition of paragraph 138, wherein the three oxidoreductases are a peroxidase and two laccases.

[144] The enzyme composition of paragraph 138, wherein the three oxidoreductases are a catalase and two peroxidases.

[145] The enzyme composition of paragraph 138, wherein the three oxidoreductases are a laccase and two peroxidases.

[146] The enzyme composition of paragraph 138, wherein the three oxidoreductases are three catalases.

[147] The enzyme composition of paragraph 138, wherein the three oxidoreductases are three laccases.

[148] The enzyme composition of paragraph 138, wherein the three oxidoreductases are three peroxidases.

[149] The enzyme composition of any of paragraphs 121-148, which is a fermentation broth formulation or a cell composition.

[150] The enzyme composition of any of paragraphs 121-149, which further comprises one or more non-ionic and/or cationic surfactants.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A process for degrading a cellulosic material, comprising: treating the cellulosic material with an enzyme composition in the presence of a combination of an AA9 polypeptide and one or more oxidoreductases selected from the group consisting of a catalase, a laccase, and a peroxidase.

2. The process of claim 1, further comprising recovering the degraded cellulosic material.

3. The process of claim 2, wherein the degraded cellulosic material is a sugar.

4. A process for producing a fermentation product, comprising:
(a) saccharifying a cellulosic material with an enzyme composition in the presence of a combination of an AA9 polypeptide and one or more oxidoreductases selected from the group consisting of a catalase, a laccase, and a peroxidase;
(b) fermenting the saccharified cellulosic material with one or more fermenting microorganisms to produce the fermentation product; and
(c) recovering the fermentation product from the fermentation.

5. The process of claim 4, wherein steps (a) and (b) are performed simultaneously in a simultaneous saccharification and fermentation.

6. A process of fermenting a cellulosic material, comprising: fermenting the cellulosic material with one or more fermenting microorganisms, wherein the cellulosic material is saccharified with an enzyme composition in the presence of a combination of an AA9 polypeptide and one or more oxidoreductases selected from the group consisting of a catalase, a laccase, and a peroxidase.

7. The process of claim 6, wherein the fermenting of the cellulosic material produces a fermentation product.

8. The process of claim 7, further comprising recovering the fermentation product from the fermentation.

9. The process of claim 1, wherein the cellulosic material is pretreated before saccharification.

10. The process of claim 1, wherein the combination of the AA9 polypeptide and the one or more oxidoreductases is the AA9 polypeptide and one oxidoreductase selected from the group of a catalase, a laccase, and a peroxidase.

11. The process of claim 1, wherein the combination of the AA9 polypeptide and the one or more oxidoreductases is the AA9 polypeptide and two oxidoreductases independently selected from the group of catalases, laccases, and peroxidases.

12. The process of claim 11, wherein the two oxidoreductases are a catalase and a laccase; a catalase and a peroxidase; a laccase and a peroxidase; two catalases; two laccases; or two peroxidases.

13. The process of claim 1, wherein the combination of the AA9 polypeptide and the one or more oxidoreductases is the AA9 polypeptide and three oxidoreductases independently selected from the group of catalases, laccases, and peroxidases.

14. The process of claim 13, wherein the three oxidoreductases are a catalase, a laccase, and a peroxidase; a laccase and two catalases; a peroxidase and two catalases; a catalase and two laccases; a peroxidase and two laccases; a catalase and two peroxidases; a laccase and two peroxidases; three catalases; three laccases; or three peroxidases.

15. The process of claim 1, wherein the enzyme composition comprises one or more enzymes selected from the group consisting of a cellulase, a hemicellulase, an esterase, an expansin, a ligninolytic enzyme, a pectinase, a protease, and a swollenin.

16. The process of claim 1, wherein the presence of the combination of the AA9 polypeptide and the one or more oxidoreductases synergistically increases the hydrolysis of the cellulosic material by the enzyme composition at least 1.01-fold compared to the AA9 polypeptide alone, the one or more oxidoreductases alone, or absence of the AA9 polypeptide and the one or more oxidoreductases.

17. The process of claim 1, wherein oxygen is added during the degradation or saccharification of the cellulosic material to maintain a concentration of dissolved oxygen in the range of 0.5 to 10% of the saturation level.

18. (canceled)

19. (canceled)

20. The process of claim 4, wherein the cellulosic material is pretreated before saccharification.

21. The process of claim 6, wherein the cellulosic material is pretreated before saccharification.
